(19) United States
(12) Patent Application Publication
(10) Pub. No.: US 2006/0281900 A1
(43) Pub. Date: Dec. 14, 2006

(54) Lepidoptera voltage-gated calcium channels

(76) Inventors: Shilan Wu, Edison, NJ (US); Jon H Hayashi, Princeton Junction, NJ (US); Lyle P. Kinne, Morrisville, PA (US); Peter M Dierks, Yardley, PA (US); Connie Perretz Dierks, Yardley, PA (US)

Correspondence Address: FMC Corporation;Patent Administrator, 1735 Market Street, Philadelphia, PA, 19103, US

(21) Appl. No.: 10/534,279

(22) PCT Filed: Nov. 10, 2003
(86) PCT No.: PCT/US03/36001

§ 371 Date: Apr. 21, 2006

Related U.S. Application Data
(60) Provisional application No. 60/425,077, filed on Nov. 8, 2002.

(51) Int. Cl. C07K 14/705; G01N 33/567; C07H 21/04; C12P 21/06; A01N 25/00; C07K 16/28; C12N 5/06

(52) U.S. Cl. 530/350; 435/69.1; 435/320.1; 435/348; 536/23.5; 530/388.22; 435/7.2; 514/2

(57) ABSTRACT

Novel nucleic acid sequences encoding lepidoptera calcium channel subunits, and recombinant expressions and host cells comprising the same are disclosed. Isolated lepidoptera calcium channel subunits, host cells expressing a lepidoptera calcium channel subunits, methods of producing the lepidoptera calcium channel subunits and antibodies specific for the lepidoptera calcium channel subunits are also disclosed. Methods of identifying modulators and/or inhibitors of lepidoptera calcium channels are disclosed

### FIELD OF THE INVENTION

The present invention relates to nucleotide sequences that are useful in agrochemical, veterinary or pharmaceutical fields. In particular, the invention relates to polypeptides and nucleotide sequences that encode polypeptides that are useful in the identification or development of compounds with activity as pesticides or as pharmaceuticals.

### BACKGROUND OF THE INVENTION

Voltage-gate calcium channels play an important role in regulating physiological responses. Namely, it is suspected that depolarization of voltage-gated calcium channels results in the passage of calcium through the channel's pore thus triggering a variety of physiological response, including muscle contraction and neurotransmitter secretion (Jeziorski et al., J. Exper. Bio., 203, pp. 841-856 (2000); Edwin W. McCleskey, Current Opinion in Neurobiology, 4, pp. 304-312 (1994); Herman Moreno Davila, Annals New York Academy of Sciences, pp. 102-117; Stephen W. Jones, J. of Bioenergetics and Biomembranes, Vol. 30, No. 4 pp. 299-312 (1998); Hofmann et al. Anna. Rev. Neurosci., 17, pp. 399-418 (1994); Edward Perez-Reyes and Toni Schneider, Kidney International, Vol. 48, pp. 1111-1124 (1995); and AstraZeneca Pharmaceuticals Presentation, The 28^{th} National Medicinal Chemistry Symposium of the American Chemical Society, (June 2002)). As such, there is a desire to develop ways to target this channel as a means of identifying biologically active compounds, including insecticides (Mintz et al., Nature, Vol. 355, pp. 827-829 (1992); Wang et al., Nature Structural Biology, Vol. 7, No. 6, pp. 505-512 (2000); Wang et al., Eur. J. Biochem., 264, pp. 488-494 (1999); and Fletcher et al., Nature Structural Biology, Vol. 4, No. 7, pp. 559-566 (1997)).

Voltage-gated calcium channels have been expressed and cloned from vertebrate species, for example, humans, rabbits, rats, mice, and Discopyge ommata (Velicelebi et al., Methods in Enzymology, Vol. 294, pp. 20-47 (1999); Herman Moreno Davila, Annals New York Academy of Sciences, pp. 102-117; Hofmann et al. Anna. Rev. Neurosci., 17, pp. 399-418 (1994); Edward Perez-Reyes and Toni Schneider, Kidney International, Vol. 48, pp. 1111-1124 (1995); AstraZeneca Pharmaceuticals Presentation, The 28^{th} National Medicinal Chemistry Symposium of the American Chemical Society, June 2002; Home et al., Proc. Natl. Acad. Sci., Vol. 90, pp. 3787-3791 (1993); Rousset et al., J. of Physiology, 532, 3, pp. 583-593 (2001); Neelands et al, J. Physiology, pp. 2933-2944 (2000); and Brust et al., Neuropharmacolgy, Vol. 32, No. 11, pp. 1089-1102 (1993)). Voltage-gated calcium channels have also been expressed and cloned from invertebrate species, for example, Caenorhabditis elegans, Styllphora pistilla, Bdelloura candida, Cyanea capillata, Loligo bleeker, Aplysia californica, Drosophila melanogaster, Musca domestica, Blatella germanica, and Halocynthia roretz (Jeziorski et al., J. Exper. Bio., 203, pp. 841-856 (2000)).

### SUMMARY OF THE INVENTION

One embodiment of the invention relates to nucleotide sequences that encode or may be used to express amino acid sequences that are useful in the identification or development of compounds with (potential) activity as pesticides or as pharmaceuticals. These nucleotide sequences, including mutants and fragments thereof, which will be further described below, will also be referred to herein as “nucleotide sequences of the invention”.

Another embodiment of the invention relates to the amino acid sequences—such as proteins or polypeptides—that are encoded by, or that may be obtained by suitable expression of, the nucleotide sequences of the invention. These amino acid sequences, including mutants and fragments thereof, which will be further described below, will also be referred to herein as “amino acid sequences of the invention”.

Yet another embodiment of the invention relates to the use of the nucleotide sequences of the invention, preferably in the form of a suitable genetic construct as described below, in the transformation of host cells or host organisms, for example for the expression of the amino acid sequences of the invention. The invention also relates to host cells or host organisms that have been transformed with the nucleotide sequences of the invention or that can express the amino acid sequences of the invention.

In still yet another embodiment, the invention relates to methods for the identification or development of compounds that can modulate and/or inhibit the biological activity of the amino acid sequences of the invention, in which the above mentioned nucleotide sequences, amino acid sequences, genetic constructs, host cells or host organisms are used. Such methods, which will usually be in the form of an assay or screen, will also be further described below.

In yet another embodiment, the invention relates to compounds that can modulate the (biological activity of), or that can otherwise interact with, an amino acid sequence of the invention, either in vitro or preferably (also) in vivo. The invention also relates to compositions that contain such compounds, and to the use of such compounds in the preparation of these compositions and the control of pests.

### Definitions

Collectively, the nucleic acids of the present invention will be referred to herein as “nucleic acids of the invention”. Also, where appropriate in the context of the further description of the invention below, the terms “nucleotide sequence of the invention” and “nucleic acid of the invention” may be considered essentially equivalent and essentially interchangeable.

Also, for the purposes of the present invention, a nucleic acid is considered to be “(in) essentially isolated form)”—for example, from its native biological source—when it has been separated from at least one other nucleic acid molecule and sequence with which it is usually associated. Similarly, a polypeptide is considered to be “(in) essentially isolated (form)”—for example, from its native biological source—when it has been effectively separated from other polypeptide molecules with which it is normally associated with. In particular, a nucleic acid or polypeptide is considered “essentially isolated” when it has been purified at least 2-fold, in particular at least 10-fold, more in particular at least 100-fold, and up to 1000-fold or more.

### BRIEF DESCRIPTION OF THE DRAWINGS

FIG. 1A and FIG. 1B are schematic representation of the low copy number plasmid vector pGreen1S(+)Lox2(Xba). The multiple cloning site is located in the small region between the Kpn I and Sac I restriction sites and is flanked by promoters for T3 and T7 RNA polymerases. p15A, arrow denotes the position of the low copy number p15A plasmid origin of DNA replication. loxp, arrows mark the locations of 34 bp repeats used in site-specific recombination by cre recombinase. AmpR, arrow marks the position and orientation of the β-lactamase gene, which confers resistance to the antibiotics ampicillin and carbenicillin. incA, marks the location of a DNA segment whose presence blocks the replication of the P1 plasmid expressing cre recombinase in E. coli strain DH10B(ZIP). f1(+) origin, arrow marks the location and orientation of the origin for rolling circle replication of the filamentous f1 phage. FIG. 1A: Depicts the structure of the circular form of the plasmid prior to insertion into a derivative of bacteriophage λgt11. FIG. 1B: Depicts the structure of the linearized form of the plasmid, as it exists in the bacteriophage cloning vector λgtGreen1S. pGreen1S(+)Lox2(Xba) was cleaved with restriction enzymes Mfe I and Pme I and the large linear fragment was inserted between the Sac I and Eco RI sites (which were destroyed) in a derivative of bacteriophage λgt11. Digestion of λgtGreen1S with Xho I and Sac I produces two phage arms which can be used for cDNA library construction.

FIG. 2 shows currents generated in response to voltage steps in Xenopus oocytes injected with the insect α1 subunit and the TBW β-2 subunit. The plot of the relationship between peak current and voltage. Peak current was determined between markers 1 and 2 (inverted triangles).

FIG. 3 shows currents generated in response to voltage steps in Xenopus oocytes injected with the insect α1 subunit, TBW β-2 subunit and cotton aphid α2δ subunit. The plot of the relationship between peak current and voltage is shown. Peak current was determined between markers 1 and 2 (inverted triangles).

FIG. 4 shows currents generated in response to voltage steps in Xenopus oocytes injected with the insect α1 subunit, TBW β-2 subunit and TBW α2δ-2 subunit. The plot of the relationship between peak current and voltage is shown. Peak current was determined between markers 1 and 2 (inverted triangles.

### DETAILED DESCRIPTION OF THE INVENTION

The present invention was established from the finding that the amino acid sequences of the invention can be used as (potential) “target(s)” for in vitro or in vivo interaction with chemical compounds and other factors (with the term “target” having its usual meaning in the art, provide for example the definition given in WO 98/06737, which is incorporated herein by reference). Consequently, compounds or factors that have been identified as interacting with the amino acid sequences of the invention (e.g. by the methods as described herein below) may be useful as active agents in the agrochemical, veterinary or pharmaceutical fields.

In one embodiment, the invention relates to a nucleic acid, preferably in (essentially) isolated form, which nucleic acid comprises a nucleotide sequence of the invention, and in particular the nucleotide sequence of SEQ ID NO: 1. The nucleotide sequence of SEQ ID NO: 1 was derived or isolated from the Heliothis virescens organism, in the manner as further described in the Examples below. This sequence provides an incomplete coding sequence for the Heliothis virescens Voltage-gated Calcium channel subunit α1.

In another embodiment, the invention relates to a nucleic acid, preferably in (essentially) isolated form, which nucleic acid comprises a nucleotide sequence of the invention, and in particular the nucleotide sequence of SEQ ID NO: 5. The nucleotide sequence of SEQ ID NO: 5 is a chimeric sequence in which the Heliothis virescens Voltage-gated Calcium channel subunit α1 sequence of SEQ ID NO: 1 is provided with a stop codon from nucleotides 1 to 429 listed in SEQ ID NO: 3 derived from cotton aphid cDNA library (Example 1). The aphid-derived sequences are from Aphid Voltage-gated Calcium channel subunit α1. Construction of this sequence is further described in the Examples below. This sequence provides a functional chimeric coding sequence.

In another embodiment, the invention relates to a nucleic acid, preferably in (essentially) isolated form, which nucleic acid comprises a nucleotide sequence of the invention, and in particular the nucleotide sequence of SEQ ID NO: 7, SEQ ID NO: 9, or SEQ ID NO: 11. The nucleotide sequences of SEQ ID NO: 7, SEQ ID NO: 9, or SEQ ID NO: 11 were derived or isolated from the Heliothis virescens organism, such as in the manner as further described in the Examples below. These sequences provide complete coding sequences for the Heliothis virescens Voltage-gated Calcium channel subunits β-1, β-2, and β-3, respectively.

In another embodiment, the invention relates to a nucleic acid, preferably in (essentially) isolated form, which nucleic acid comprises a nucleotide sequence of the invention, and in particular the nucleotide sequence of SEQ ID NO: 13. The nucleotide sequence of SEQ ID NO: 13 was derived or isolated from the Aphis gossypii organism, in the manner as further described in the Examples below. This sequence provides an complete coding sequence for the Aphis gossypii Voltage-gated Calcium channel subunit α2δ.

In another embodiment, the invention relates to a nucleic acid, preferably in (essentially) isolated form, which nucleic acid comprises a nucleotide sequence of the invention, and in particular the nucleotide sequence of SEQ ID NO: 15 or SEQ ID NO: 17. The nucleotide sequence of SEQ ID NO: 15 or SEQ ID NO: 17 were derived or isolated from the Heliothis virescens organism, such as in the manner as further described in the Examples below. These sequences provide complete coding sequences for the Heliothis virescens voltage-gated Calcium channel subunits α2δ-1 and α2δ-2, respectively.

Generally, the nucleotide sequences of the invention, when in the form of a nucleic acid, may be DNA or RNA, and may be single stranded or double stranded. For example, the nucleotide sequences of the invention may be genomic DNA, cDNA or synthetic DNA (such as DNA with a codon usage that has been specifically adapted for expression in the intended host cell or host organism, which may for instance be designed using suitable computer programs such as the BackTranslate analysis tool in Vector NTI (InforMax, Inc., Bethesda, Md.). Thus, the nucleotide sequences of the invention may contain intron sequences, and also generally comprises different splice variants.

Yet another embodiment relates to a double stranded RNA molecule directed against a nucleotide sequence of the invention (one strand of which will usually comprise at least part of a nucleotide sequence of the invention). Such double-stranded RNA molecules have particular utility in RNA interference studies of gene function. (Zamore, et al., Cell 101:25-33 (2000)). The invention also relates to genetic constructs that can be used to provide such double stranded RNA molecules (e.g. by suitable expression in a host cell or host organism, or for example in a bacterial strain such as E. coli). For such constructs, reference is made to Maniatis et al., Molecular Cloning, a Laboratory Manual (Cold Spring Harbor Press, 1989).

In a broader sense, the term “nucleotide sequence of the invention” also refers to:
parts or fragments of the nucleotide sequence of SEQ ID NO: 1, SEQ ID NO:5, SEQ ID NO:7, SEQ ID NO:9, SEQ ID NO:11, SEQ ID NO 13, SEQ ID NO:15, and SEQ ID NO:17;
(natural or synthetic) mutants, variants, alleles, analogs, orthologs (herein below collectively referred to as “mutants”) of the nucleotide sequence of SEQ ID NO: 1, SEQ ID NO:5, SEQ ID NO:7, SEQ ID NO:9, SEQ ID NO:11, SEQ ID NO 13, SEQ ID NO:15, and SEQ ID NO:17 as further described below.
parts or fragments of such (natural or synthetic) mutants;
nucleotide fusions of the nucleotide sequence of SEQ ID NO: 1, SEQ ID NO:5, SEQ ID NO:7, SEQ ID NO:9, SEQ ID NO:11, SEQ ID NO 13, SEQ ID NO:15, and SEQ ID NO:17 (or a part or fragment thereof) with at least one further nucleotide sequence;
nucleotide fusions of (natural or synthetic) mutants (or a part or fragment thereof) with at least one further nucleotide sequence;
   in which such mutants, parts, fragments or fusions are preferably as further described below.

The invention also comprises different splice variants of the above nucleotide sequences.

Preferably, a nucleotide sequence of the invention will have a length of at least 500 nucleotides, preferably at least 1,000 nucleotides, more preferably at least 2,000 nucleotides; and up to a length of at most 8,000 nucleotides, preferably at most 7,500 nucleotides, more preferably at most, 7,000 nucleotides.

Examples of parts or fragments of the nucleotide sequence of SEQ If) NO: 1; or a part or fragment of a (natural or synthetic) mutant thereof include, but are not limited to, 5′ or 3′ truncated nucleotide sequences, or sequences with an introduced in frame start codon or stop codon. Also, two or more such parts or fragments of one or more nucleotide sequences of the invention may be suitably combined (e.g. ligated in frame) to provide a further nucleotide sequence of the invention.

Preferably, any such parts or fragments will be such that they comprise at least one continuous stretch of at least 100 nucleotides, preferably at least 250 nucleotides, more preferably at least 500 nucleotides, even more preferably more than 1,000 nucleotides, of the nucleotide sequence of SEQ ID NO: 1, SEQ ID NO:5, SEQ ID NO:7, SEQ ID NO:9, SEQ ID NO:11, SEQ ID NO 13, SEQ ID NO:15, and SEQ ID NO:17. In the case of SEQ ID NO:5, fragments according to the invention comprise at least 5, preferably 10, more preferably 20 or more nucleotides from SEQ ID NO:5.

Also, it is expected that—based upon the disclosure herein—the skilled person will be able to identify, derive or isolate natural “mutants” (as mentioned above) of the nucleotide sequence of SEQ ID NO: 1, SEQ ID NO:5, SEQ ID NO:7, SEQ ID NO:9, SEQ ID NO:11, SEQ ID NO 13, SEQ ID NO:15, and SEQ ID NO:17 from (other individuals of) the same species (for example from an individual of a different strain or line, including but not limited to mutant strains or lines). It is also expected that—based upon the disclosure herein—the skilled person will be able to provide or derive synthetic mutants (as defined herein above) of the nucleotide sequence of SEQ ID NO: 1.

In one specific embodiment, the mutant is such that it encodes the nucleotide sequence of SEQ ID NO: 1, SEQ ID NO:5, SEQ ID NO:7, SEQ ID NO:9, SEQ ID NO:11, SEQ ID NO 13, SEQ ID NO:15, or SEQ ID NO:17 or a part or fragment thereof.

Preferably, any mutants as described herein will have one or more, and preferably all, of the structural characteristics or conserved features referred to below for the nucleotide sequences of SEQ ID NO: 1, SEQ ID NO:5, SEQ ID NO:7, SEQ ID NO:9, SEQ ID NO:11, SEQ ID NO 13, SEQ ID NO:15, or SEQ ID NO:17.

In particular, any mutants, parts or fragments as described herein may be such that they at least encode the active or catalytic site of the corresponding amino acid sequence of the invention and a binding domain of the corresponding amino acid sequence of the invention.

Also, any mutants, parts or fragments as described herein will preferably have a degree of “sequence identity”, at the nucleotide level, with the nucleotide sequence of SEQ ID NO 1, SEQ ID NO:5, SEQ ID NO:7, SEQ ID NO:9, SEQ ID NO:11, SEQ ID NO 13, SEQ ID NO:15, or SEQ ID NO:17, of at least 75%, preferably at least 80%, more preferably at least 85%, and in particular more than 90%, and up to 95% or more.

Also, preferably, any mutants, parts or fragments of the nucleotide sequence of the invention will be such that they encode an amino acid sequence which has a degree of “sequence identity”, at the amino acid level, with the amino acid sequence of SEQ ID NO: 2, SEQ ID NO:6, SEQ ID NO:8, SEQ ID NO:10, SEQ ID NO:12, SEQ ID NO 14, SEQ ID NO:16, or SEQ ID NO:18, of at least 80%, and in particular more than 90% and up to 95% or more, in which the percentage of “sequence identity” is calculated as described below.

For this purpose, the percentage of “sequence identity” between a given nucleotide sequence and the nucleotide sequence of SEQ ID NO: 1, SEQ ID NO:5, SEQ ID NO:7, SEQ ID NO:9, SEQ ID NO:11, SEQ ID NO 13, SEQ ID NO:15, or SEQ ID NO:17 may be calculated by dividing the number of nucleotides in the given nucleotide sequence that are identical to the nucleotide at the corresponding position in the nucleotide sequence of SEQ ID NO: 1, SEQ ID NO:5, SEQ ID NO:7, SEQ ID NO:9, SEQ ID NO:11, SEQ ID NO 13, SEQ ID NO:15, or SEQ ID NO:17 by the total number of nucleotides in the given nucleotide sequence and multiplying by 100%, in which each deletion, insertion, substitution or addition of a nucleotide—compared to the sequence of SEQ ID NO:1, SEQ ID NO:5, SEQ ID NO:7, SEQ ID NO:9, SEQ ID NO:11, SEQ ID NO 13, SEQ ID NO:15, or SEQ ID NO:17—is considered as a difference at a single nucleotide position.

The preferred computer program for performing global sequence alignments and determining sequence identity is clustalW. (Higgins, et al., Nucl. Acids Res. 22:4673-4680 (1994)), which is publically available for a variety of computer platforms. Preferably the paramenters for the clustalW program for protein sequence alignments are ktuple=1, diagonals=5, windows=5, gap=3, score=PERCENTAGE, matrix=BLOSUM, open penalty=10.0 and extension penalty=0.05.

Also, in a preferred aspect, any mutants, parts or fragments as described herein will encode proteins or polypeptides having biological activity that is essentially similar to the biological activity described above for the sequences of SEQ ID NO: 1, SEQ ID NO:5, SEQ ID NO:7, SEQ ID NO:9, SEQ ID NO:11, SEQ ID NO 13, SEQ ID NO:15, or SEQ ID NO:17, i.e. to a degree of at least 50%, preferably at least 75%, and up to 90%, as measured by standard assay techniques as described below.

Any mutants, parts or fragments as described herein are preferably such that they are capable of hybridizing with the nucleotide sequence of SEQ ID NO: 1, SEQ ID NO:5, SEQ ID NO:7, SEQ ID NO:9, SEQ ID NO:11, SEQ ID NO 13, SEQ ID NO:15, or SEQ ID NO:17, i.e. under conditions of “high stringency”. Such conditions will be clear to the skilled person, for example from the standard handbooks, such as Sambrook et al. and Ausubel et al., mentioned above, as well as in EP 0 967 284, EP 1 085 089 or WO 00/55318, which are incorporated herein by reference.

It is also within the scope of the invention to use a fusion of a nucleotide sequence of the invention (as described above) with one or more further nucleotide sequence(s), including but not limited to one or more coding sequences, non-coding sequences or regulatory sequences. Preferably, in such fusions, the one or more further nucleotide sequences are operably connected (as described below) to the nucleotide sequence of the invention (for example so that, when the further nucleotide sequence is a coding sequence, the nucleotide fusion encodes a protein fusion as described below).

In another embodiment, the invention relates to an antisense molecule against a nucleotide sequence of the invention.

The nucleic acids of the invention may also be in the form of a genetic construct, again as further described below. Genetic constructs of the invention will generally comprise at least one nucleotide sequence of the invention, optionally linked to one or more elements of genetic constructs known per se, as described below. Such genetic constructs may be DNA or RNA, and are preferably double-stranded DNA. The constructs may also be in a form suitable for transformation of the intended host cell or host organism, in a form suitable for integration into the genomic DNA of the intended host cell or in a form suitable independent replication, maintenance and inheritance in the intended host organism. For instance, the genetic construct may be in the form of a vector, such as for example a plasmid, cosmid, a yeast artificial chromosome (“YAC”), a viral vector or transposon. In particular, the vector may be an expression vector, i.e. a vector that can provide for expression in vitro or in vivo (e.g. in a suitable host cell or host organism as described below). An expression vector comprising a nucleotide sequence of the invention is also referred to herein as a recombinant expression vector. These constructs will also be referred to herein as “genetic constructs of the invention”.

In a preferred embodiment, such a construct in a recombinant expression vector which will comprise:
a) the nucleotide sequence of the invention; operably connected to:
b) one or more regulatory elements, such as a promoter and optionally a suitable terminator;
   and optionally also:
c) one or more further elements of genetic constructs known per se; in which the terms “regulatory element”, “promoter”, “terminator”, “further elements” and “operably connected” have the meanings indicated herein below.

As the one or more “further elements” referred to above, the genetic construct(s) of the invention may generally contain one or more suitable regulatory elements (such as a suitable promoter(s), enhancer(s), or terminator(s)), 3′- or 5′-untranslated region(s) (“UTR”) sequences, leader sequences, selection markers, expression markers or reporter genes, or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and the transformation technique to be used.

Preferably, in the genetic constructs of the invention, the one or more further elements are “operably linked” to the nucleotide sequence(s) of the invention or to each other, by which is generally meant that they are in a functional relationship with each other. For instance, a promoter is considered “operably linked” to a coding sequence if said promoter is able to initiate or otherwise control or regulate the transcription or the expression of a coding sequence (in which said coding sequence should be understood as being “under the control of” said promoter)

Generally, when two nucleotide sequences are operably linked, they will be in the same orientation and usually also in the same reading frame. They will usually also be essentially contiguous, although this may also not be required.

Preferably, the optional further elements of the genetic construct(s) used in the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling or regulating the transcription or the expression of a nucleotide sequence—e.g. a coding sequence—to which it is operably linked (as defined above).

Such a promoter may be a constitutive promoter or an inducible promoter, and may also be such that it (only) provides for expression in a specific stage of development of the host cell or host organism, or such that it (only) provides for expression in a specific cell, tissue, organ or part of a multicellular host organism.

Some particularly preferred promoters include, but are not limited to, constitutive promoters, such as cytomegalovirus (“CMV”), Rous sarcoma virus (“RSV”), simian virus-40 (“SV40”), for example, pSVL SV40 Late Promoter Expression Vector (Pharmacia Biotech Inc., Piscataway, N.J.), or herpes simplex virus (“HSV”) for expression in mammalian cells or insect constitutive promoters such a the immediate early baculovirus promoter described by Jarvis et al. (Methods in Molecular Biology Vol. 39 Baculovirus Expression Protocols, ed. C. Richardson, Hamana Press Inc., Totowa, N.J. (1995) available in pIE vectors from Novagen (Novagen, Inc. Madison, Wis.) or insect inducible promoters such as the Drosophila metallothionein promoter described by Bunch et al. (Nucleic Acids Research, Vol. 6, No. 3 1043-106, (1988)) available in vectors from Invitrogen (Invitrogen Corporation, Carlsbad, Calif.).

A selection marker should be such that it allows—i.e. under appropriate selection conditions—host cells or host organisms that have been (successfully) transformed with the nucleotide sequence of the invention to be distinguished from host cells or organisms that have not been (successfully) transformed. Some preferred, but non-limiting examples of such markers are genes that provide resistance against antibiotics (such as geneticin or G-418 (GIBCO-BRL, Grand Island, N.Y.), kanamycin or ampicillin), genes that provide for temperature resistance, or genes that allow the host cell or host organism to be maintained in the absence of certain factors, compounds or (food) components in the medium that are essential for survival of the non-transformed cells or organisms.

A leader sequence should be such that—in the intended host cell or host organism—it allows for the desired post-translational modifications or such that it directs the transcribed mRNA to a desired part or organelle of a cell such as a signal peptide. A leader sequence may also allow for secretion of the expression product from said cell. As such, the leader sequence may be any pro-, pre-, or prepro-sequence operable in the host cell or host organism, including, but not limited to, picornavirus leaders, potyvirus leaders, a human immunoglobulin heavy-chain binding protein (“BiP”), a tobacco mosaic virus leader (“TMV”), and a maize chlorotic mottle virus leader (“MCMV”).

An expression marker or reporter gene should be such that—in the host cell or host organism—it allows for detection of the expression of (a gene or nucleotide sequence present on) the genetic construct. An expression marker may optionally also allow for the localization of the expressed product, e.g. in a specific part or organelle of a cell or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins, such as GFP, antibody recognition proteins, for example, V5 epitope or poly Histidine available in vectors and antibodies supplied by Invitrogen, or purification affinity handles such as polyhistidine which allows for purification on nickel columns or dihydrofolate reductase which allows for purification on methotrexate column, or markers which allow for selection of cells expressing the gene such as the E. coli beta-galactosidase gene.

For some non-limiting examples of the promoters, selection markers, leader sequences, expression markers and further elements that may be present or used in the genetic constructs of the invention—such as terminators, transcriptional or translational enhancers or integration factors—reference is made to the general handbooks such as Sambrook et al. and Ausubel et al. mentioned above, to W. B. Wood et al., “The nematode Caenorhabditis elegans”, Cold Spring Harbor Laboratory Press (1988) and D. L. Riddle et al., “C. ELEGANS II”, Cold Spring Harbor Laboratory Press (1997), as well as to the examples that are given in WO 95/07463, WO 96/23810, WO 95/07463, WO 95/21191, WO 97/11094, WO 97/42320, WO 98/06737, WO 98/21355, U.S. Pat. No. 6,207,410, U.S. Pat. No. 5,693,492 and EP 1 085 089, which are each incorporated herein by reference. Other examples will be clear to the skilled person.

Another embodiment of the invention relates to a host cell or host organism that has been transformed or contains one or more nucleotide sequences, one or more nucleic acids or one or more genetic constructs of the invention. The invention also relates to a host cell or host organism that expresses, or (at least) is capable of expressing (e.g. under suitable conditions), one or more amino acid sequences of the invention. In some embodiments, the host cell may comprises a recombinant vector that expresses Voltage-gated Calcium channel chimeric α1, TBW β-1 and/or β-2 and/or β-3 and aphid α2δ and/or TBW α2δ-1 and/or α2δ-2 subunits, including functional fragments and chimerics, to form a functional Voltage-gated Calcium channel wherein at least one of α1, β and α2δ subunits is a chimeric α1, TBW β-1 and/or β-2 and/or β-3 and TBW α2δ-1 and/or α2δ-2 and/or aphid α2δ subunits of the invention, preferably SEQ ID NO:5, SEQ ID NO:7, SEQ ID NO:9, SEQ ID NO:11, SEQ ID NO 13, SEQ ID NO:15 and/or SEQ ID NO:17. In some preferred embodiments, the host cell comprises a recombinant vector that expresses Voltage-gated Calcium channel α1, β and α2δ subunits, including functional fragments and chimerics, to form a functional Voltage-gated Calcium channel wherein at least two of chimeric α1, TBW β-1 and/or β-2 and/or β-3 and TBW α2δ-1 and/or α2δ-2 subunits of the invention, preferably SEQ ID NO:5, SEQ ID NO:7, SEQ ID NO:9, SEQ ID NO:11, SEQ ID NO 13, SEQ ID NO:15 and/or SEQ ID NO:17. In some preferred embodiments, the host cell comprises a recombinant vector that expresses α1, β and α2δ subunits from coding sequences that are nucleic acid molecule of the invention, chimeric α1, plus TBW β-1 and/or β-2 and/or β-3 plus TBW α2δ-1 and/or α2δ-2 and/or aphid α2δ subunits of the invention, preferably SEQ ID NO:5, plus one or more of SEQ ID NO:7, SEQ ID NO:9, SEQ ID NO:11, SEQ ID NO 13 plus one or more of SEQ ID NO:15 and/or SEQ ID NO:17. Collectively, such host cells or host organisms will also be referred to herein as “host cells or host organisms of the invention”.

The host cell may be any suitable (fungal, prokaryotic or eukaryotic) cell or cell line, for example:
a bacterial strain, including but not limited to strains of E. coli, Bacillus, Streptomyces and Pseudomonas;
a fungal cell, including but not limited to cells from species of Aspergillus and Trichoderma;
a yeast cell, including but not limited to cells from species of Kluyveromyces or Saccharomyces;
an amphibian cell or cell line, such as Xenopus oocytes.

In one specific embodiment, which may particularly useful when the nucleotide sequences of the invention are (to be) used in the discovery and development of insecticidal compounds, the host cell may be an insect-derived cell or cell line, such as:
cells or cell lines derived from lepidoptera, including but not limited to Spodoptera SF9 and Sf21 cells,
cells or cell lines derived from Aphis;
cells or cell lines derived from Drosophila, such as Schneider and Kc cells; and
cells or cell lines derived from a pest species of interest (as mentioned below), such as from Heliothis virescens.

The host cell may also be a mammalian cell or cell line, including but not limited to CHO- and BHK-cells and human cells or cell lines such as HEK, HeLa and COS.

The host organism may be any suitable multicellular (vertebrate or invertebrate) organism, including but not limited to:
a nematode, including but not limited to nematodes from the genus Caenorhabditis, such as C. elegans,
an insect, including but not limited to species of Aphis, Drosophila, Heliothis, or a specific pest species of interest (such as those mentioned above);
other well known model organisms, such as zebrafish;
a mammal such as a rat or mouse;

Other suitable host cells or host organisms will be clear to the skilled person, for example from the handbooks and patent applications mentioned above.

It should be noted that when a nucleotide sequence of the invention is expressed in a multicellular organism, it may be expressed throughout the entire organism, or only in one or more specific cells, tissues, organs or parts thereof, for example by expression under the control of a promoter that is specific for said cell(s), tissue(s), organ(s) or part(s).

The nucleotide sequence may also be expressed during only a specific stage of development or life cycle of the host cell or host organism, again for example by expression under the control of a promoter that is specific for said stage of development or life cycle. Also, as already mentioned above, said expression may be constitutive, transient or inducible.

Preferably, these host cells or host organisms are such that they express, or are (at least) capable of expressing (e.g. under suitable conditions), an amino acid sequence of the invention (and in case of a host organism: in at least one cell, part, tissue or organ thereof). The invention also includes further generations, progeny and offspring of the host cell or host organism of the invention, which may for instance be obtained by cell division or by sexual or asexual reproduction.

In yet another aspect, the invention relates to a nucleic acid, preferably in (essentially) isolated form, which nucleic acid encodes or can be used to express an amino acid sequence of the invention (as defined herein), and in particular the amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18.

The amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18 may be isolated from the species mentioned above, using any technique(s) for protein isolation and purification known to one skilled in the art. Alternatively, the amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18 may be obtained by suitable expression of a suitable nucleotide sequence—such as the nucleotide sequence of SEQ ID NO: 1, SEQ ID NO:5, SEQ ID:7, SEQ ID NO:9, SEQ ID:11, SEQ ID NO 13, SEQ ID NO:15 and SEQ ID NO:17, respectively, or a suitable mutant thereof—in an appropriate host cell or host organism, as further described below.

In another aspect, the invention relates to a protein or polypeptide, preferably in (essentially) isolated form, said protein or polypeptide comprising an amino acid sequence of the invention (as defined above), in particular the amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18.

In a broader sense, the term “amino acid sequence of the invention” also comprises:
parts or fragments of the amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18;
(natural or synthetic) mutants, variants, alleles, analogs, orthologs (herein below collectively referred to as “analogs”) of the amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18;
parts or fragments of such analogs;
fusions of the amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18 (or a part or fragment thereof) with at least one further amino acid residue or sequence;
fusions of the amino acid sequence of an analog (or a part or fragment thereof) with at least one further amino acid residue or sequence;
   in which such mutants, parts, fragments or fusions are preferably as further described below.

The term “amino acid sequence of the invention” also comprises “immature” forms of the above-mentioned amino acid sequences, such as a pre-, pro- or prepro-forms or fusions with suitable leader sequences. Also, the amino acid sequences of the invention may have been subjected to post-translational processing or be suitably glycosylated, depending upon the host cell or host organism used to express or produce said amino acid sequence; or may be otherwise modified (e.g. by chemical techniques known per se in the art).

Examples of parts or fragments of the amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18, or a part or fragment of a (natural or synthetic) analog thereof mutant thereof include, but are not limited to, N- and C-truncated amino acid sequence. Also, two or more parts or fragments of one or more amino acid sequences of the invention may be suitably combined to provide an amino acid sequence of the invention.

Preferably, an amino acid sequence of the invention has a length of at least 100 amino acids, preferably at least 250 amino acids, more preferably at least 350 amino acids; and up to a length of at most 2,500 amino acids, preferably at most 2,000 amino acids, more preferably at most 1,750 amino acids.

Preferably, any such parts or fragments will be such that they comprise at least one continuous stretch of at least 5 amino acids, preferably at least 10 amino acids, more preferably at least 20 amino acids, even more preferably more than 30 amino acids, of the amino acid sequence of SEQ ID NO: 2, SEQ ID NO:6, SEQ ID:8, SEQ ID NO:10, SEQ ID:12, SEQ ID NO 14, SEQ ID NO:16 and SEQ ID NO:18. In the case of fragments of SEQ ID NO:6, the fragment must include at least 4 amino acids from SEQ ID NO:2, preferably 8 or more, more preferably 15 or more.

In particular, any parts or fragments as described herein are such that they (at least) comprise the active or catalytic site of the corresponding amino acid sequence of the invention or a binding domain of the corresponding amino acid sequence of the invention. As will be clear to the skilled person, such parts or fragments may find particular use in assay- and screening techniques (as generally described below) and (when said part or fragment is provided in crystalline form) in X-ray crystallography.

Also, it is expected that—based upon the disclosure herein—the skilled person will be able to identify, derive or isolate natural “analogs” (as mentioned above) of the amino acid sequence of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18. Such mutants could be derived from (other individuals of) the same species (for example from an individual of a different strain or line, including but not limited to mutant strains or lines); or from (individuals of) other species. For example, such analogs could be derived from the insect species mentioned above.

It is also expected that—based upon the disclosure herein—the skilled person will be able to provide or derive synthetic “analogs” (as mentioned above) of the amino sequence of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18.

Preferably, any mutants as described herein will have one or more, and preferably all, of the structural characteristics or conserved features referred to below for the sequences of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18.

Preferably, any analogs, parts or fragments as described herein will be such that they have a degree of “sequence identity”, at the amino acid level, with the amino acid sequence of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18 of at least 80%, and in particular more than 90% and up to 95% or more.

For this purpose, the percentage of “sequence identity” between a given amino acid sequence and the amino acid sequence of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18 may be calculated by dividing the number of amino acid residues in the given amino acid sequence that are identical to the amino acid residue at the corresponding position in the amino acid sequence of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18 by the total number of amino acid residues in the given amino acid sequence and multiplying by 100%, in which each deletion, insertion, substitution or addition of an amino acid residue—compared to the sequence of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18—is considered as a difference at a single amino acid (position). As mentioned above, the preferred method of performing pairwise sequence alignments for such calcualtions is with the program clustalW.

Also, preferably, any analogs, parts or fragments as described herein will have a biological activity that is essentially similar to the biological activity described above for the sequences of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18, i.e. to a degree of at least 10%, preferably at least 50% more preferably at least 75%, and up to 90%, as measured by standard assay techniques as described below.

It is also within the scope of the invention to use a fusion of an amino acid sequence of the invention (as described above) with one or more further amino acid sequences, for example to provide a protein fusion. Generally, such fusions may be obtained by suitable expression of a suitable nucleotide sequence of the invention—such as a suitable fusion of a nucleotide sequence of the invention with one or more further coding sequences—in an appropriate host cell or host organism, as further described below.

One particular embodiment, such fusions may comprise an amino acid sequence of the invention fused with a reporter protein such as glutathione S-transferase (“GST”), green fluorescent protein (“GFP”), luciferase or another fluorescent protein moiety. As will be clear to the skilled person, such fusions may find particular use in expression analysis and similar methodologies.

In another embodiment, the fusion partner may be an amino acid sequence or residue that may be used in purification of the expressed amino acid sequence, for example using affinity techniques directed against said sequence or residue. Thereafter, said sequence or residue may be removed (e.g. by chemical or enzymatical cleavage) to provide the nucleotide sequence of the invention (for this purpose, the sequence or residue may optionally be linked to the amino acid sequence of the invention via a cleavable linker sequence). Some preferred, but non-limiting examples of such residues are multiple histidine residues and glutatione residues.

In one preferred, but non-limiting aspect, any such fusion will have a biological activity that is essentially similar to the biological activity described above for the sequences of SEQ ID NO: 2, SEQ ID NO: 6, SEQ ID: 8, SEQ ID NO: 10, SEQ ID: 12, SEQ ID NO: 16 or SEQ ID NO: 18, i.e. to a degree of at least 10%, preferably at least 50% more preferably at least 75%, and up to 90%, as measured by standard assay techniques as described below.

The nucleotide sequences and amino acid sequences of the invention may generally be characterized by the presence of one or more of the following structural characteristics or conserved features:

For the gene Heliothis virescens: SEQ ID NO: 1 is a cDNA sequence encompassing the open reading frame; and SEQ ID NO: 2 is the protein encoded by SEQ ID NO: 1. The Heliothis N-type voltage-gated calcium channel protein sequence is related to other N-type voltage-gated calcium channel proteins as set forth in the table below, where the relatedness values were determined using clustalW and the parameters set forth above.

| | Percentage Identity |
|---|---|
| Sequence: | Full length |
| Drosophila CCAA-DROME¹ | 77 |
| C. Elegans UNC-2² | 61 |
| Heliothis virescens (SEQ ID NO: 2) | 100 |

¹Genebank Accession No. AAC47406, Smith, L. A. et al., J. Neurosci. 16 (24), 7868-7879, 1996.
²Genebank Accession No. AAB36868, Waterston, R., Science 282 (5396), 2012-2018, 1998.

By analogy to other calcium channels, it is likely that the functional protein is monomeric. See, e.g., Hannan and Hall, In Comparative Molecular Neurobiology, Y. Pichon, 1993, Birkhuaser Verlag Basel Switzerland.

On the basis of the above, and although the invention is not specifically limited to any specific explanation or mechanism, the nucleotide sequences and amino acid sequences have (biological) activity as a voltage-gated calcium channel. In particular, the present invention has shown activity as a voltage-gated calcium channel from insects of the order Lepidoptera, for example, borers, cutworms, armyworms, tobacco budworms, fruit worms, cabbage worms, moths, and loppers, preferably tobacco budworms, and true bugs that have mouthparts adapted to piercing and sucking.

As is known in the art, biological activity of this kind can be measured using standard assay techniques, for example, through competition with a labeled, known ligand for binding sites; by measuring calcium flux using calcium fluorescent dyes (see Velicelebi et al.) by fluorescent assays based on protein interactions such as fluorescence resonance energy transfer, time resolved fluorescence or fluorometric or colorimetric reporter assays; or any technology suitable for assaying voltage-gated calcium channels. Preferably, the biological activity is measured by measuring calcium flux using calcium fluorescent dyes.

Another embodiment of the invention relates to a nucleic acid probe that is capable of hybridizing with a nucleotide sequence of the invention under conditions of moderate stringency, preferably under conditions of high stringency, and in particular under stringent conditions (all as described above). Such nucleotide probes may for instance be used for detecting or isolating a nucleotide sequence of the invention or as a primer for amplifying a nucleotide sequence of the invention; all using techniques known per se, for which reference is again made to the general handbooks such as Sambrook et al. and Ausubel et al., mentioned above.

Preferably, when to be used for detecting or isolating another nucleotide sequence of the invention, such a nucleotide probe will usually have a length of between 15 and 100 nucleotides, and preferably between 20 and 80 nucleotides. When used as a primer for amplification, such a nucleotide probe will have a length of between 25 and 75 nucleotides, and preferably between 20 and 40 nucleotides.

Generally, such probes can be designed by the skilled person starting from a nucleotide sequence or amino acid sequence of the invention—and in particular the sequence of SEQ ID NO: 1 or SEQ ID NO: 2, SEQ ID NO: 5 or SEQ ID: 6, SEQ ID NO: 7 or SEQ ID: 8, SEQ ID NO: 9 or SEQ ID: 10, SEQ ID NO: 11 or SEQ ID NO: 12, SEQ ID NO 13 or SEQ ID NO 14, or SEQ ID NO: 15 or SEQ ID NO: 16, and SEQ ID: 17 or SEQ ID NO: 18—optionally using a suitable computer algorithm. Also, as will be clear to the skilled person, such probes may be degenerate probes.

In a further aspect, the invention relates to methods for preparing mutants and genetic constructs of the nucleotide sequences of the present invention.

Natural mutants of the nucleotide sequences of the present invention may be obtained in a manner essentially analogous to the method described in the Examples, or alternatively by:
construction of a DNA library from the species of interest in an appropriate expression vector system, followed by direct expression of the mutant sequence;
construction of a DNA library from the species of interest in an appropriate expression vector system, followed by screening of said library with a probe of the invention (as described below) or with a nucleotide sequence of the invention;
isolation of mRNA that encodes the mutant sequence from the species of interest, followed by cDNA synthesis using reverse transcriptase;
   or by any other suitable method(s) or technique(s) known per se, for which reference is for instance made to the standard handbooks, such as Sambrook et al., “Molecular Cloning: A Laboratory Manual” (2nd. ed.), Vols. 1-3, Cold Spring Harbor Laboratory Press (1989) and F. Ausubel et al., “Current protocols in molecular biology”, Green Publishing and Wiley Interscience, New York (1987).

Techniques for generating such synthetic sequences of the nucleotide sequences of the present invention will be clear to the skilled person and may for instance include, but are not limited to, automated DNA synthesis; site-directed mutagenesis; combining two or more parts of one or more naturally occurring sequences, introduction of mutations that lead to the expression of a truncated expression product; introduction of one or more restriction sites (e.g. to create cassettes or regions that may easily be digested or ligated using suitable restriction enzymes), and the introduction of mutations by means of a PCR reaction using one or more “mismatched” primers, using for example a sequence of a naturally occurring voltage-gated calcium channel as a template. These and other techniques will be clear to the skilled person, and reference is again made to the standard handbooks, such as Sambrook et al. and Ausubel et al., mentioned above.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors include:
vectors for expression in mammalian cells: pSVL SV40 (Pharmacia), pMAMneo (Clontech), pcDNA3 (Invitrogen), pMC1neo (Stratagene), pSG5 (Stratagene), pMSG (Pharmacia), pIND (Invitrogen), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565);
vectors for expression in bacteria cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacII (Invitrogen), pEI1 Novagen), pMT/V5His (Invitrogen).

In a further aspect, the invention relates to methods for transforming a host cell or a host organism with a nucleotide sequence, with a nucleic acid or with a genetic construct of the invention. The invention also relates to the use of a nucleotide sequence, of a nucleic acid or of a genetic construct of the invention transforming a host cell or a host organism.

According to one specific embodiment, the expression of a nucleotide sequence of the invention in a host cell or host organism may be reduced, compared to the original (e.g. native) host cell or host organism. This may for instance be achieved in a transient manner using antisense or RNA-interference techniques well known in the art, or in a constitutive manner using random, site specific or chemical mutagenesis of the nucleotide sequence of the invention.

Suitable transformation techniques will be clear to the skilled person and may depend on the intended host cell or host organism and the genetic construct to be used. Some preferred, but non-limiting examples of suitable techniques include ballistic transformation, (micro-)injection, transfection (e.g. using suitable transposons), electroporation and lipofection. For these and other suitable techniques, reference is again made to the handbooks and patent applications mentioned above.

After transformation, a step for detecting and selecting those host cells or host organisms that have been successfully transformed with the nucleotide sequence or genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

In yet another aspect, the invention relates to methods for producing an amino acid sequence of the invention.

To produce or obtain expression of the amino acid sequences of the invention, a transformed host cell or transformed host organism may generally be kept, maintained or cultured under conditions such that the (desired) amino acid sequence of the invention is expressed or produced. Suitable conditions will be clear to the skilled person and will usually depend upon the host cell or host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell or host organism used. Also, the amino acid sequence of the invention may be glycosylated, again depending on the host cell or host organism used.

The amino acid sequences of the invention may then be isolated from the host cell or host organism or from the medium in which said host cell or host organism was cultivated, using protein isolation and purification techniques known per se, such as (preparative) chromatography and electrophoresis techniques, differential precipitation techniques, affinity techniques (e.g. using a specific, cleavable amino acid sequence fused with the amino acid sequence of the invention) and preparative immunological techniques (i.e. using antibodies against the amino acid sequence to be isolated).

In one embodiment, the amino acid sequence thus obtained may also be used to generate antibodies specifically against said sequence or an antigenic part or epitope thereof.

In one embodiment, the present invention relates to antibodies, for example monoclonal and polyclonal antibodies, that are generated specifically against amino acid sequences of the present invention, preferably SEQ ID NO: 2, SEQ ID NO: 6, SEQ ID: 8, SEQ ID NO: 10, SEQ ID NO: 12, SEQ ID NO 14, SEQ ID NO: 16 or SEQ ID NO: 18 or analogs, variants, alleles, orthologs, parts, fragments or epitopes thereof provided that antibodies specific for SEQ ID NO: 6 do not cross react with aphid α1 such as SEQ ID NO: 4.

Such antibodies, which form a further aspect of the invention, may be generated in a manner known per se, for example as described in GB-A-2 357 768, U.S. Pat. No. 5,693,492, WO 95/32734, WO 96/23882, WO 98/02456, WO 98/41633 and WO 98/49306. Often, but not exclusively, such methods will involve as immunizing a immunocompetent host with the pertinent amino acid sequence of the invention or an immungenic part thereof (such as a specific epitope), in amount(s) and according to a regimen such that antibodies against said amino acid sequence are raised, and than harvesting the antibodies thus generated, e.g. from blood or serum derived from said host.

For instance, polyclonal antibodies can be obtained by immunizing a suitable host such as a goat, rabbit, sheep, rat, pig or mouse with (an epitope of) an amino acid sequence of the invention, optionally with the use of an immunogenic carrier (such as bovine serum albumin or keyhole limpet hemocyanin) or an adjuvant such as Freund's, saponin, aluminium hydroxide or a similar mineral gel, or keyhole limpet hemocyanin or a similar surface active substance. After a suitable immune response has been raised (usually within 1-7 days), the antibodies can be isolated from blood or serum taken from the immunized animal in a manner known per se, which optionally may involve a step of screening for an antibody with desired properties (i.e. specificity) using known immunoassay techniques, for which reference is again made to for instance WO 96/23882.

Monoclonal antibodies may for example be produced using continuous cell lines in culture, including hybridoma-based and similar techniques, again essentially as described in the above cited references. Accordingly, cells and cell lines that produce monoclonal antibodies against an amino acid sequence of the invention form a further aspect of the invention, as do methods for producing antibodies against amino acid sequences of the invention, which methods may generally involve cultivating such a cell and isolating the antibodies from the culture or medium, again using techniques known per se.

Also, Fab-fragments against the amino acid sequences of the invention (such as F(ab)₂, Fab′ and Fab fragments) may be obtained by digestion of an antibody with pepsin or another protease, reducing disulfide-linkages and treatment with papain and a reducing agent, respectively. Fab-expression libraries may for instance be obtained by the method of Huse et al., 1989, Science 245:1275-1281.

In another embodiment, the amino acid sequence of the invention, or a host cell or host organism comprising a recombinant expression vector that encodes and expresses such an amino acid sequence, may also be used to identify or develop compounds or other factors that can modulate the (biological) activity of, or that can otherwise interact with, the amino acid sequences of the invention, and such uses form further aspects of the invention. As will be clear to the skilled person, in this context, the amino acid sequence of the invention will serve as a target for interaction with such a compound or factor.

In this context, the terms “modulate”, “modulation, “modulator” and “target” will have their usual meaning in the art, for which reference is inter alia made to the definitions given in WO 98/06737. Generally, a modulator is a compound or factor that can enhance, inhibit or reduce or otherwise alter, influence or affect (collectively referred to as “modulation”) a functional property of a biological activity or process (for example, the biological activity of an amino acid sequence of the invention).

In this context, the amino acid sequences of the invention may serve as a target for modulation in vitro (e.g. as part of an assay or screen) or for modulation in vivo (e.g. for modulation by a compound or factor that is known to modulate the target, which compound or factor may for example be used as an active compound for agrochemical, veterinary or pharmaceutical use).

For example, the amino acid sequences, host cells or host organisms of the invention may be used as part of an assay or screen that may be used to identify or develop modulators of the amino acid sequence of the invention, such as a primary screen (e.g. a screen used to identify modulators of the target from a set or library of test chemicals with unknown activity with respect to the target) or a secondary assay (e.g. an assay used for validating hits from a primary screen or used in optimizing hit molecules, e.g. as part of hits-to-leads chemistry).

For instance, such an assay or screen may be configured as an in vitro assay or screen, which will generally involve binding of the compound or factor to be tested as a potential modulator for the target (herein below also referred to as “test chemical”) to the target, upon which a signal generated by said binding is measured. Suitable techniques for such in vitro screening will be clear to the skilled person, and are for example described in Eldefrawi et al., (1987). FASEB J., Vol. 1, pages 262-271 and Rauh et al., (1990), Trends in Pharmacol. Sci., vol. 11, pages 325-329. For example, such an assay or screen may be configured as a binding assay or screen, in which the test chemical is used to displace a detectable ligand from the target (e.g. a radioactive or fluorescent ligand), upon which the amount of ligand displaced from the target by the modulator is determined.

Such an assay or screen may also be configured as a cell-based assay or screen, in which a host cell of the invention is contacted with or exposed to a test chemical, upon which at least one biological response by the host cell is measured.

Also, such an assay or screen may also be configured as an whole animal screen, in which a host organism of the invention is contacted with or exposed to a test chemical, upon which at least one biological response (such as a phenotypical, behavioral or physiological change, including but not limited to paralysis or death) by the host organism is measured,

Thus, generally, the assays and screens described above will comprise at least one step in which the test chemical is contacted with the target (or with a host cell or host organism that expresses the target), and in particular in such a way that a signal is generated that is representative for the modulation of the target by the test chemical. In a further step, said signal may then be detected.

Accordingly, in one aspect, the invention relates to a method for generating a signal that is representative for the interaction of an amino acid sequence of the invention with a test chemical, said method at least comprising the steps of:
a) contacting an amino acid sequence of the invention, or a host cell or host organism containing or expressing an amino acid sequence, with said test chemical, in such a way that a signal may be generated that is representative for the interaction between said test chemical and said amino acid sequence; and optionally
b) detecting the signal that may thus be generated.

In another aspect, the invention relates to a method for identifying modulators and/or inhibitors of an amino acid sequence of the invention (e.g. from a set or library of test chemicals), said method at least comprising the steps of:
a) contacting an amino acid sequence of the invention, or a host cell or host organism containing or expressing an amino acid sequence, with a test chemical, in such a way that a signal may be generated that is representative for the interaction between said test chemical and said the target; and optionally
b) detecting the signal that may thus be generated, said signal identifying the modulator and/or inhibitor of said amino acid sequence.

Accordingly, the present invention provides methods of identifying a modulator of a lepidoptera calcium channel protein activity. The methods comprise the step of performing a test assay by contacting a test cell, which comprises a recombinant expression vector that contains nucleic acid sequences that encodes a functional calcium channel in which at least one of the nucleic acid sequences is a nucleic acid sequence of the invention and that expresses the calcium channel with a solution containing calcium in the presence of a test compound, and detecting the amount of intracellular calcium in the test cell. In preferred embodiments, the functional calcium channel is made up of two or more amino acid sequences of the invention. In some embodiments, the functional calcium channel is made up of three amino acid sequences of the invention, preferably, SEQ ID NO: 6, one of SEQ ID: 8, SEQ ID NO: 10 or SEQ ID: 12, and one of SEQ ID NO 14, SEQ ID NO: 16 or SEQ ID NO: 18. The methods also comprise the step performing a negative control assay by contacting a negative control cell which comprises a recombinant expression vector that contains a nucleic acid sequence that encodes the lepidoptera calcium channel and expresses lepidoptera calcium channel, with a solution containing calcium in the absence of the test compound, and detecting the amount of intracellular calcium in the negative control cell. The amount of intracellular calcium in the test cell is compared to the amount of intracellular calcium in the negative control cell. A change in the amount of calcium in the test cell compared to the amount of intracellular calcium in the negative control cell indicates the test compound is a modulator of a lepidoptera calcium channel protein activity. In some preferred embodiments, the test cell is a CHO or HEK cell. In some preferred embodiments, the intracellular calcium is detected by using an assay in which fluorescence generated by dye inside of said cell interacting with intracellular calcium is measured. In some preferred embodiments, the methods further comprise performing a positive control assay by contacting a positive control cell which comprises a recombinant expression vector that contains a nucleic acid sequence that encodes the lepidoptera calcium channel and expresses lepidoptera calcium channel, with a solution containing calcium in the absence of the test compound and in the presence of a lepidoptera calcium channel agonist, and detecting the amount of intracellular calcium in said positive control cell. In some embodiments, the methods further comprise the step of either performing a second-type negative control assay by contacting a lepidoptera calcium channel negative control cell that does not express lepidoptera calcium channel with a solution containing calcium in the absence of the test compound, and detecting the amount of calcium taken up by said lepidoptera calcium channel negative control cell; and/or performing a third-type negative control assay by contacting a lepidoptera calcium channel negative control cell that does not express lepidoptera calcium channel with a solution containing calcium in the absence of the test compound and in the presence of a lepidoptera calcium channel agonist, and detecting the amount of calcium taken up by said lepidoptera calcium channel negative control cell. In preferred embodiments, the lepidoptera calcium channel protein used in the methods has an amino acid sequence selected from the group consisting of SEQ ID NO: 2 or SEQ ID NO: 6, a mutant thereof, and a fragment thereof, which may be combined with one or more additional subunits selected from the group SEQ ID: 8, SEQ ID NO: 10, SEQ ID: 12, SEQ ID NO 14, SEQ ID NO: 16 and SEQ ID NO: 18 a mutant thereof, and a fragment thereof. In some embodiments, the nucleic acid sequence that encodes the lepidoptera calcium channel is SEQ ID NO: 1 or SEQ ID NO: 5, with auxiliary subunits, when present, being encoded by SEQ ID: 7, SEQ ID NO: 9, SEQ ID: 11, SEQ ID NO 13, SEQ ID NO: 15 or SEQ ID NO: 17.

According to some embodiments, methods of identifying an inhibitor of a lepidoptera calcium channel protein activity comprise the step of performing a test assay by contacting a test cell, which comprises a recombinant expression vector that contains a nucleic acid sequence that encodes the lepidoptera calcium channel and expresses lepidoptera calcium channel, with a solution containing calcium and a lepidoptera calcium channel agonist in the presence of a test compound, and detecting the amount of intracellular calcium in said test cell. A control assay is also performed by contacting a negative control cell, which comprises a recombinant expression vector that contains a nucleic acid sequence that encodes the lepidoptera calcium channel and expresses lepidoptera calcium channel, with a solution containing calcium and a lepidoptera calcium channel agonist in the absence of the test compound, and detecting the amount of intracellular calcium in said negative control cell. The amount of intracellular calcium in the test cell is compared to the amount of intracellular calcium in the control cell. A decrease in the amount of intracellular calcium in the test cell compared to the amount of intracellular calcium in the control cell indicates the test compound is an inhibitor of lepidoptera calcium channel protein activity. In some preferred embodiments, the test cell is a CHO or HEK cell. In some preferred embodiments, the intracellular calcium is detected by using an assay in which fluorescence generated by dye inside of the cell interacting with intracellular calcium is measured. In some preferred embodiments, the lepidoptera calcium channel protein has an amino acid sequence selected from the group consisting of SEQ ID NO: 2 or SEQ ID NO: 6, a mutant thereof and a fragment thereof, which may be combined with one or more additional subunits selected from the group SEQ ID: 8, SEQ ID NO: 10, SEQ ID: 12, SEQ ID NO 14, SEQ ID NO: 16 and SEQ ID NO: 18, a mutant thereof, and a fragment thereof. In some embodiments, the nucleic acid sequence that encodes said lepidoptera calcium channel is SEQ ID NO: 1 or SEQ ID NO: 5 with auxiliary subunits, when present, encoded by, SEQ ID: 7, SEQ ID NO: 9, SEQ ID NO: 11, SEQ ID NO 13, SEQ ID NO: 15 and SEQ ID NO: 17.

A test chemical may be part of a set or library of compounds, which may be a diverse set or library or a focused set or library, as will be clear to the skilled person. The libraries that may be used for such screening can be prepared using combinatorial chemical processes known in the art or conventional means for chemical synthesis.

The assays and screens of the invention may be carried out at medium throughput to high throughput, for example in an automated fashion using suitable robotics. In particular, in this embodiment, the method of the invention may be carried out by contacting the target with the test compound in a well of a multi-well plate, such as a standard 24, 96, 384, 1536 or 3456 well plate.

Usually, in a screen or assay of the invention, for each measurement, the target or host cell or host organism will be contacted with only a single test compound. However, it is also within the scope of the invention to contact the target with two or more test compounds—either simultaneously or sequentially—for example to determine whether said combination provides a synergistic effect.

Once a test chemical has been identified as a modulator and/or inhibitor for an amino acid sequence of the invention (e.g. by means of a screen or assay as described hereinabove), it may be used per se as a modulator and/or inhibitor of the relevant amino acid sequence of the invention, preferably, an amino acid sequence of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18, mutants thereof, and fragments thereof, more preferably SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18 (e.g. as an active substance for agrochemical, veterinary or pharmaceutical use), or it may optionally be further optimized for final use, e.g. to improve properties such as solubility, adsorption, bio-availability, toxicity, stability, persistence, environmental impact, etc. It will be clear to the skilled person that the nucleotide sequences, preferably SEQ ID NO: 1, SEQ ID NO: 5, SEQ ID: 7, SEQ ID NO: 9, SEQ ID: 11, SEQ ID NO 13, SEQ ID NO: 15 or SEQ ID NO: 17, amino acid sequences, host cells or host organisms and methods of the invention may find further use in such optimization methodology, for example as (part of) secondary assays.

The invention is not particularly limited to any specific manner or mechanism in or via which the modulator and/or inhibitor (e.g. the test chemical, compound or factor) modulates, inhibits, or interacts with, the target (in vivo or in vitro). For example, the modulator and/or inhibitor may be an agonist, an antagonist, an inverse agonist, a partial agonist, a competitive inhibitor, a non-competitive inhibitor, a cofactor, an allosteric inhibitor or other allosteric factor for the target, or may be a compound or factor that enhances or reduces binding of target to another biological component associated with its (biological) activity, such as another protein or polypeptide, a receptor, or a part of organelle of a cell. As such, the modulator and/or inhibitor may bind with the target (at the active site, at an allosteric site, at a binding domain or at another site on the target, e.g. covalently or via hydrogen bonding), block and/or inhibit the active site of the target (in a reversible, irreversible or competitive manner), block and/or inhibit a binding domain of the target (in a reversible, irreversible or competitive manner), or influence or change the conformation of the target.

As such, the test chemical, modulator and/or inhibitor may for instance be:
an analog of a known substrate of the target;
an oligopeptide, e.g. comprising between 2 and 20, preferably between 3 and 15 amino acid residues;
an antisense or double stranded RNA molecule;
a protein, polypeptide;
a cofactor or an analog of a cofactor.

The test chemical, modulator and/or inhibitor may also be a reference compound or factor, which may be a compound that is known to modulate, inhibit or otherwise interact with the target (e.g. a known substrate or inhibitor for the target) or a compound or factor that is generally known to modulate, inhibit or otherwise interact with other members from the general class to which the target belongs (e.g. a known substrate or inhibitor of said class).

Preferably, however, the test chemical, modulator and/or inhibitor is a small molecule, by which is meant a molecular entity with a molecular weight of less than 1500, preferably less than 1000. This may for example be an organic, inorganic or organometallic molecule, which may also be in the form or a suitable salt, such as a water-soluble salt. The term “small molecule” also covers complexes, chelates and similar molecular entities, as long as their (total) molecular weight is in the range indicated above.

As already mentioned above, the compounds or factors that have been identified or developed as modulators and/or inhibitors of the amino acid sequences of the invention, preferably, an amino acid sequence of SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18, mutants thereof, and fragments thereof, more preferably SEQ ID NO: 2, SEQ ID NO: 6, SEQ ID: 8, SEQ ID NO: 10, SEQ ID: 12, SEQ ID NO 14, SEQ ID NO: 16 or SEQ ID NO: 18, (and precursors for such compounds) may be useful as active substances in the agrochemical, veterinary or pharmaceutical fields, for example in the preparation of agrochemical, veterinary or pharmaceutical compositions, and both such modulators as well as compositions containing them further aspects of the invention.

For example, in the agrochemical field, the modulators and/or inhibitors of the invention may be used as an insecticide, nematicide, molluscide, helminticide, acaricide or other types of pesticides or biocides, e.g. to prevent or control (infestations with) harmful organisms, both as contact agents and as systemic agents. As such, the modulators and/or inhibitors may for example be used as a crop protection agent, as a pesticide for household use, or as an agent to prevent or treat damage caused by harmful organisms (e.g. for the protection of seed, wood or stored crops or fruits). Preferably, the modulators and/or inhibitors of the invention are used as insecticides For any such application, one or more modulators and/or inhibitors of the invention may be suitably combined with one or more agronomically acceptable carriers, adjuvants or diluents—and optionally also with one or more further compounds known per se with activity as (for example) a plant protection agent (to broaden the spectrum of action and optionally to provide a synergistic effect), herbicide, fertilizer or plant growth regulator—to provide a formulation suitable for the intended final use. Such a formulation may for example be in the form of a solution, emulsion, dispersion, concentrate, aerosol, spray, powder, flowable, dust, granule, pellet, fumigation candle, bait or other suitable solid, semi-solid or liquid formulation, and may optionally also contain suitable solvents, emulsifiers, stabilizers, surfactants, antifoam agents, wetting agents, spreading agents, sticking agents, attractants or (for a bait) food components. Reference is made to the standard manuals, such as “Pesticidal Formulation Research”, ACS-publications (1969) and “Pesticide Formulations”, Wade van Valkenburg Ed, Marcel Dekker publications (1973).

Such compositions may generally contain one or more modulators and/or inhibitors of the invention in a suitable amount, which generally may be between 0.1 and 99%, and in particular between 10 and 50%, by weight of the total composition.

The modulators and/or inhibitors and compositions of the invention may be particularly useful as insecticides, for example to combat or control undesired or harmful insects (both adult and immature forms, such as larvae) from following orders:
Coleoptera, such as Pissodes strobi, Diabrotica undecimpunctata howardi, and Leptinotarsa decemlinieata;
Diptera, such as Rhagoletis pomonella, Mayetiola destructor, and Liriomyza huidobrensis;
Hymenoptera, such as Neodiprion taedae tsugae, Camponotus pennsylvanicus, and Solenopsis wagneri;
Hemiptera, such as Pseudatomoscelis seriatus, Lygus lineolaris (Palisot de Beauvois), and Acrosternum hilare;
Homoptera and Aphis, such as Aphis gossypii; and
Lepidoptera such as Heliothis virescens.

When used to control harmful or undesired organisms, these organisms may be directly contacted with the modulators, inhibitors, or compositions of the invention in an amount suitable to control (e.g. kill or paralyze) the organism. This amount may be readily determined by the skilled person (e.g. by testing the compound on the species to be controlled) and will usually be in the region of between particular between 10 and 500 g/ha, in particular between 100 and 250 g/ha.

The modulators, inhibitors, or compositions of the invention may also be applied systemically (e.g. to the habitat of the organism to be controlled or to the soil), and may also be applied to the plant, seed, fruit etc. to be protected, again in suitable amounts, which can be determined by the skilled person. The modulators and/or inhibitors of the invention may also be incorporated—e.g. as additives—in other compositions known per se, for example to replace other pesticidal compounds normally used in such compositions.

In one specific embodiment, the modulators and/or inhibitors and compositions of the invention may be used in the fields of agrochemical, veterinary or human health to prevent or treat infection or damage or discomfort caused by parasitic organisms, and in particular by parasitic arthropods, nematodes and helminths such as:
ectoparasitic arthropods such as ticks, mites, fleas, lice, stable flies, horn flies, blowflies and other biting or sucking ectoparasites;
endoparasites organisms such as helminths;
   and also to prevent or treat diseases that are caused or transferred by such parasites. For such purposes, the modulators and/or inhibitors of the invention may for example be formulated as a tablet, an oral solution or emulsion, an injectable solution or emulsion, a lotion, an aerosol, a spray, a powder, a dip or a concentrate.

In the fields of animal and human health, the modulators, inhibitors, and compositions of the invention may also be used for the prevention or treatment of diseases or disorders in which the amino acid sequence of the invention may be involved as a target. For this purpose, the modulators and/or inhibitors of the invention may be formulated with one or more additives, carriers or diluents acceptable for pharmaceutical or veterinary use, which will be clear to the skilled person.

Thus, in a further aspect, the invention relates to the use of a modulator and/or inhibitor of the invention in the preparation of a composition for agrochemical, veterinary or pharmaceutical use, as described hereinabove. The invention relates to the use of the modulators, inhibitors and compositions of the invention in controlling harmful organisms and in preventing infestation or damage caused by harmful organisms, again as described above.

The invention will now be further illustrated by means of the following non-limiting Examples.

### EXAMPLES

### Example 1

### Construction of cDNA Libraries and Sequence Databases

TABLE I

Oligonucleotides used in cDNA
library construction

| | |
|---|---|
| Primer | Sequence |

| | |
|---|---|
| SacdT | GAGAGAGAGAAGAGCTCT₁₆VN (SEQ ID NO:19) |
| XhoAd4 | TCGAGCTGAGGTACGAACGC (SEQ ID NO:20) |
| XhoAd5 | PGCGTTCGTACCTCAGC (SEQ ID NO:21) |

cDNA libraries enriched for full-length clones were constructed by Invitrogen Corporation (Carlsbad, Calif.) from poly(A)-containing RNA purified from (a) mixed life stage cotton aphids (cotton aphid cDNA library), and (b) dissected heads from late instar Heliothis virescens larvae (TBW head cDNA library). Both libraries were constructed in plasmid vector pCMV-SPORT6.1 and transformed into E. coli strain DH10B(TonA). The libraries were stored without further amplification as glycerol cultures at −80° C. A portion of the cotton aphid cDNA library was amplified, normalized and arrayed in 384 well plates by Invitrogen Corporation. The arrayed clones were subjected to automated sequence analysis using vector primers that flanked the 5′ and 3′ termini of the cloned cDNA (Genome Therapeutics, Waltham, Mass.). The sequence reads were trimmed for quality and vector contamination, assembled into contigs and installed into a BLAST-compatible database. Access to the Basic Local Alignment Sequence Tool (BLAST) suite of search tools and instructions for the use of these tools and the construction of BLAST-compatible databases can be obtained from the National Center for Biotechnology Information at the National Institutes for Health (Bethesda, Md.).

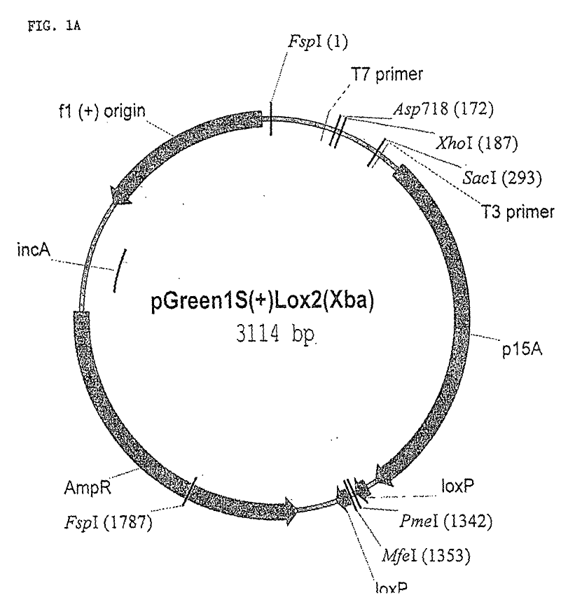

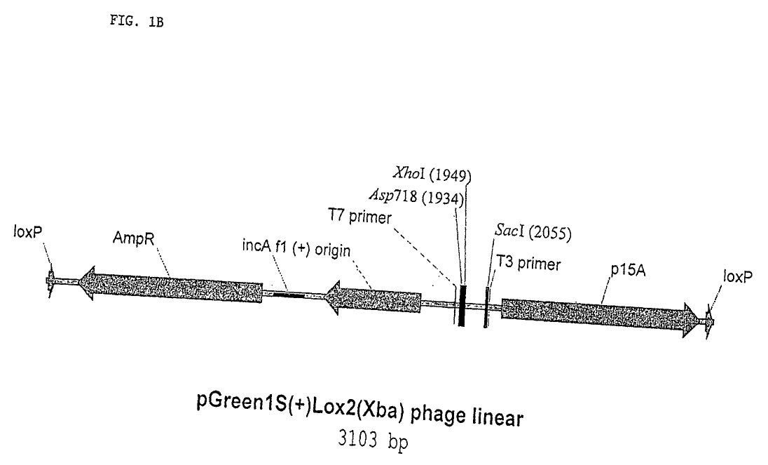

A cDNA library was additionally constructed from poly(A)-containing RNA isolated from ventral nerve cords and brains of late instar Heliothis virescens larvae (TBW nerve cord library). Approximately 195 nerve cords and brains were dissected from early 5^{th} instar TBW larvae and quick frozen on dry ice. The tissue was homogenized in 3 ml TRIZOL™ reagent (Invitrogen Corporation) at room temperature and total RNA was extracted according to the manufacturer's protocol. RNA quality was verified by TV absorption spectroscopy (320 nm-220 nm) and electrophoresis of a small glyoxylated sample on a 1% agarose gel containing 1× NorthernMax™-Gly buffer (Ambion, Austin, Tex.). Poly(A)-containing RNA was selected from the total RNA by affinity chromatography using the Oligotex® mRNA isolation kit (QIAGEN Inc., Valencia, Calif.) as described by the manufacturer. cDNA was synthesized by a modification of the procedure of Carninci and Hayashizaki (Methods in Enzymology, Vol. 303, pp. 19-44 (1999)). Briefly, 6.2 ug of poly(A)-containing RNA was mixed with 11.2 ul 80% (v/v) glycerol and 400 pmol SacdT primer (Table I) in a total volume of 24 ul. The primer/RNA mixture was heated to 65° C. for 10 min, cooled to 45° C. on a thermocycler and mixed with 76 ul of synthesis buffer, which was also pre-heated to 45° C. The final composition of the first strand cDNA synthesis reaction was: 50 mM Tris-HCl (pH 8.3 at 25° C.), 75 mM KCl, 3 mM Mg₂Cl, 10 mM dithiothreitol (DTT), 0.6 mM dGTP, 0.6 mM dATP, 0.6 mM TTP, 0.6 mM 5-methyl-dCTP, 50 ug/ml bovine serum albumin, 2000 units/ml RNaseOUT™ ribonuclease inhibitor (Invitrogen Corporation) and 20,000 units/ml SuperScript™ III reverse transcriptase (Invitrogen Corporation). The reaction was incubated at 45° C. for 2 min, slow cooled over 2 min to 35° C., incubated at 35° C. for 2 min, stepped to 50° C. and incubated for 5 min, and finally stepped to 55° C. and incubated for 60 min. The first strand reaction was terminated by adding EDTA to 10 mM, sodium N-lauroylsarcosine to 0.1% (w/v) and proteinase K (Invitrogen Corporation) to 0.2 mg/ml. The mixture was incubated at 45° C. for 15 min and then extracted once with phenol:chloroform:isoamyl alcohol (25:24:1). The aqueous phase was removed, 100 ul 5M NH₄OAc and 500 ul ethanol were added, and the cDNA was precipitated at −20° C. for 60 min. The cDNA was then collected by centrifugation, washed once with 70% ethanol, dissolved in 50 ul 0.1×TE (1 mM Tris-HCl (pH 8.0)-0.1 mM EDTA) and combined on ice with 200 ul second-strand synthesis buffer. The final composition of the second-strand reaction was 20 mM Tris-HCl (pH 8.3), 130 mM KCl, 10 mM (NH₄)₂SO₄, 5 mM MgCl₂, 10 mM DTT, 0.6 mM dATP, 0.6 mM dGTP, 0.6 mM dCTP, 0.6 mM TTP,10 mM b-NAD, 50 ug/ml BSA, 3 units E. coli RNase H (Invitrogen Corporation), 2.5 units E. coli DNA ligase (Invitrogen Corporation) and 25 units E. coli DNA polymerase I (Invitrogen Corporation). Second strand synthesis was performed at 15° C. for 150 min. The enzyme mix was heat inactivated at 70° C. for 10 min and then placed on ice. One microliter (5 units) of T4 DNA polymerase (Invitrogen Corporation) was added and the double-stranded cDNA was blunted by incubation at 15° C. for 15 min. To facilitate directional cloning a double-stranded Xho adapter was added to the blunted cDNA and then removed from the 3′ end by digestion with Sac I, which cleaves uniquely in the primer for first strand synthesis. The adapter was formed by combining 2 nmol each of oligonucleotides XhoAd4 and XhoAd5 (Table I) in 40 ul of 20 mM NaCl, heating the mixture to 70° C. for 3 min and slow cooling to 25° C. over 10 min. The annealed Xho adapter (450 pmol) was ligated with 1.7 ug double-stranded cDNA overnight at 8° C. in a 30 ul reaction containing 50 mM Tris-HCl (pH 7.5), 10 mM MgCl₂, 1 mM ATP, 10 mM DTT, 25 ug/ml BSA and 600 units T4 DNA ligase (New England Biolabs, Beverly, Mass.). The cDNA was again purified by proteinase K digestion, phenol:chloroform extraction and ethanol precipitation, as described earlier. The cDNA was then digested with Sac I at a ratio of 25 units enzyme per microgram cDNA and size-fractionated on a 2 ml Sepharose CL-2B (Sigma Chemical Co., St Louis, Mo.) gel filtration column equilibrated with 10 mM Tris-HCl (pH 8.0), 1 mM EDTA, 0.1% (w/v) SDS and 0.1 M NaCl. Peak fractions (730 ng cDNA) were pooled, concentrated by ethanol precipitation and redissolved in 10 ul 0.1×TE (pH 8.0). For cloning 50 ng cDNA was combined with 500 ng Xho I/Sac I phage arms prepared from bacteriophage λgtGreen1S in a volume of 4.4 ul containing 57 mM Tris-HCl (pH 7.5), 11.4 mM MgCl₂, 1.1 mM ATP, 11.4 mM DTT and 28 ug/ml BSA (molar ratio cDNA to vector=2:1). Three-tenths microliter (3 units) T4 polynucleotide kinase New England Biolabs) was added and the mixture was incubated at 37° C. for 15 min to phosphorylate the Xho adapter on the cDNA. The cDNA was then ligated to the vector adding 0.3 ul (120 units) T4 DNA ligase (New England Biolabs) and incubating the mixture overnight at 15° C. Bacteriophage λgtGreen1S is a derivative of λgt11 (Young and Davis, Proc. Natl. Acad. Sci. USA, Vol. 80, pp. 1194-1198, (1983)) in which (a) the Sal I-Xho fragment spanning coordinates 33245-33499 in the wild type bacteriophage lambda genome is deleted, and (b) the unique Sac I-Eco RI fragment containing the E. coli lacZ gene is removed and replaced by a linearized version of plasmid pGreen1S(+)Lox2(Xba) (FIG. 1A). pGreen1S(+)Lox2(Xba) is a derivative of pBluescript SK(−) (Stratagene, La Jolla, Calif.) in which (a) the incA gene of pZL1 (Invitrogen Corporation) is inserted between the f1 origin and the ampicillin-resistance gene, (b) the colE1 origin is removed and replaced with the low copy number p15A origin of pACYC184 (ATCC number 37033, American Type Culture Collection, Manassas, Va.), and (c) two tandemly repeated loxP sites (Sternberg and Hamilton, J. Mol. Biol., Vol. 150, pp. 467-86 (1981)) separated by unique Mfe I and Pme I restriction sites are inserted between the p15A origin and the ampicillin-resistance gene. Linearization of pGreen1S(+)Lox2(Xba) with Mfe I and Pme I produces the form of the plasmid which is inserted into λgtGreen1S (FIG. 1B). Aliquots of the ligation reaction were packaged into phage in vitro using Gigapack III Gold packaging extract (Stratagene) as described by the manufacturer. For preparation of recombinant plasmid, the packaged phages were plated on E. coli strain DH10B(Zip) (Invitrogen Corporation) and colonies were selected by growth on LB plates containing either ampicillin or carbenicillin. E. coli strain DH10B(Zip) expresses the cre recombinase and suppresses replication of bacteriophage lambda, thereby allowing the plasmid to be excised from the phage vector via cre-mediated recombination at the loxP sites flanking the pro-plasmid and establishing it as an independently replicating plasmid.

### Example 2

### Tobacco Budworm (“TBW”) Voltage-Gated Calcium Channel α1 Subunit Sequence Identifications

Materials and Methods.

TBW head polyA RNA isolation. A 0.1% solution of diethyl pyrocarbonate (“DEPC”; Aldrich Chemical Co., Inc. Milwaukee, Wis.) in water was incubated at 37° C. for about 16 hours and then autoclaved for 60 minutes. All glassware was baked for four hours at 250° C. and all bottle caps were soaked in the 0.1% DEPC solution. The microprobe of a Braun homogenizer (B. Braun Biotech International, Allentown, Pa.) was soaked in 50 mls of 100% ethanol (“EtOH”) and then run in 25 mls RNAzolB (a guanidinium hydrochloride preparation available from CINNA-BIOTECX Labs, Inc., Houston, Tex.). TBW heads were excised from 4^{th} instar tobacco budworm larvae and placed on ice in tared centrifuge tubes. After harvesting approximately 0.5 grams of TBW head material, the TBW heads were frozen at −70° C. until use. The heads were then taken up in a 4 mls of TRIZOL™ reagent and homogenized at fall speed and ambient temperature for 30 second. After this time, two mls of chloroform were added and the resulting mixture was centrifuged at 12000 g and ambient temperature in an SS34 rotor (Sorvall Products, L.P, Asheville, N.C.) for 15 minutes. At the conclusion of this period, the top aqueous layer was removed to a fresh 15 ml conical tube and an equal volume of isopropanol was added. The resulting mixture was centrifuged at 12000 g and ambient temperature in an SS34 rotor for 10 minutes. The resulting pellet was then washed with 10 mls of an aqueous 75% EtOH solution and centrifuged at 7500 g and ambient temperature in an SS34 rotor for 5 minutes. The supernatant was poured off and the final pellet was allowed to dry at ambient temperature for 5 minutes. After this period, the final pellet was dissolved in 1 ml of the 0.1% DEPC solution disclosed above. The concentration of total RNA was measured by UV spectrometry (GeneQuant from Amersham Biosciences, Piscataway, N.J.). 500 ugs of total RNA were used to isolate polyA mRNA following the protocol of the Micro-FastTrack™ 2.0 kit (Invitrogen Corp.). The concentration of polyA mRNA was measured by UV spectrometry. The polyA mRNA solution was stored at −70° C. for future use.

Synthesis of first strand cDNA. Reverse transcription was initiated by addition of Avian Myeloblastosis Virus (“AMV”) reverse transcriptase (Invitrogen Corp.) to 1.0 μg of template RNA. The reverse transcription reaction also included the following reagents contained in the Invitrogen cDNA Cycle Kit: 4 uls of 5× Reverse Transcription Buffer, 1 ul of Ribonuclease Inhibitor, 1 ul of 100 mM dNTPs, 1 ul of 80 mM Sodium Pyrophosphate and either Random Primers, Oligo(dT) primers, or gene specific primers. For 5′ and 3′ RACE reactions, the mRNA was reverse transcribed with oligo(dT) primer or random primer or gene specific primer using SMART™ RACE cDNA Amplification Kit (BD Biosciences Clontech, Palo Alto, Calif.) according to the manufacturer's instructions. The reactions were placed on a GeneAmp® 9700 thermal cycler (Applied Biosystems, Foster City, Calif.) and held for 60 minutes at 42° C. After this time, the AMV RT was inactivated by heating at 95° C. for 5 minutes followed by 5 minutes at 4° C. The FirstChoice™ RLM-RACE kit from Ambion (Austin, Tex.) was also used for 5′-RACE reactions according to the manufacturer's instructions.

PCR amplification. A 50 μl cDNA reaction was made utilizing the buffers and dNTPs supplied in an Advantage 2 PCR kit (BD Biosciences Clontech) according to the manufacturer's instructions. Amplifications utilizing degenerate primers typically employed annealing temperatures in the range of 50 to 60° C., those involving isoform-specific primers used annealing temperatures in the range of 65 to 70° C. 3′ and 5′ RACE reactions were carried out using primers and protocols supplied with a SMART™ RACE cDNA Amplification Kit (BD Biosciences Clontech) according to the manufacturer's instructions. The resulting PCR products were characterized by agarose gel electrophoresis. After secondary “nested” amplifications were carried out in the manner described above, bands were excised from NuSieve® gels (FMC Corp., Philadelphia, Pa.) and purified using a Qiagen gel extraction Kit according to the manufacture's instructions.

Primer synthesis and design. Oligonucleotides were synthesized by SeqWright Inc. (Houston, Tex.) and provided as lyophilized pellets that were dissolved in distilled water prior to use. PCR primers and probes were designed and annealing temperatures estimated using the VectorNTI suite 6.0 software from InforMax, Inc. (Bethesda, Md.) and Consensus-Degenerate Hybrid Oligonucleotide Primers Software (“CODEHOP”) from Rose et al. (Nucleic Acids Research, Vol. 26, pp. 1628-1635 (1998)).

Subcloning and sequencing. Purified PCR fragments were subcloned into pCR2.1-TOPO vector using a TOPO TA Cloning kit (Invitrogen Corp.) according to the manufacturer's instructions. The resulting pCR2.1-TOPO vector sequenced and then analyzed using VectorNTI suite 6.0 software.

Primers. The primers utilized were as shown in Table II:

TABLE II

| | | | |
|---|---|---|---|
| Primer | Sequence | Translation | Orientation |

| | | | |
|---|---|---|---|
| 1 | GGGCTACACCATCAGAATCCTGYTNTGGACNTT | GYTIRILLWTF | forward |
| | (SEQ ID NO:22) RT-PCR | (SEQ ID NO:23) | |
| | | Deg | |
| 2 | GAGTCTCTGGTCAGGTAGTCGAARTTRTCCAT | MDNFDYLTRDS | reverse |
| | (SEQ ID NO:24) RT-PCR | (SEQ ID NO:25) | |
| | | Deg | |
| 3 | GATACGAAGTAGGCGTAGGC | AYAYFV | reverse |
| | (SEQ ID NO:26) PCR WITH SP6 | (SEQ ID NO:27) | |
| 4 | CGATGAGAAGGCACACGTAG | YVCLLI | reverse |
| | (SEQ ID NO:28) NESTED PCR WITH SP6 | (SEQ ID NO:29) | |
| 5 | ATTTAGGTGACACTATAG | | forward |
| | (SEQ ID NO:30) SP6 | | |
| 6 | CTACGTGTGCCTTCTCATCG | YVCLLI | forward |
| | (SEQ ID NO:31) PCR WITH T7 | (SEQ ID NO:29) | |
| 7 | GCCTACGCCTACTTCGTATC | AYAYFV | forward |
| | (SEQ ID NO:32) NESTED PCR WITH T7 | (SEQ ID NO:27) | |
| 8 | TAATACGACTCACTATAGGG | | reverse |
| | (SEQ ID NO:33) T7 | | |
| 9 | CGTCGGGAAGATATACGCTGGACT | VGKIYAGL | forward |
| | (SEQ ID NO:34) 3′-RACE | (SEQ ID NO:35) | |
| 10 | CGTCAGCAACGTCGTCGAGATAGT | VSNVVEIV | forward |
| | (SEQ ID NO:36) NESTED 3′-RACE | (SEQ ID NO:37) | |
| 11 | CTAATACGACTCACTATAGGGCAAGCAG | | forward |
| | TGGTATCAACGCAGAGT Long | | reverse |
| | (SEQ ID NO:38) | | |
| | CTAATACGACTCACTATAGGGC Short | | |
| | (SEQ ID NO:39) UPM from BD Biosci- | | |
| | ences Clontech | | |
| 12 | AAGCAGTGGTATCAACGCAGAGT | | forward |
| | (SEQ ID NO:40) NUPM from BD Biosci- | | reverse |
| | ences Clontech | | |
| 13 | AAGTACTGGTCATCGCTCCGGAAC | KYWSSLRN | forward |
| | (SEQ ID NO:41) RT-PCR | (SEQ ID NO:42) | |
| 14 | GTTCCTTGCTATCGCTGTCGACAA | FLAIAVDN | forward |
| | (SEQ ID NO:43) NESTED RT-PCR | (SEQ ID NO:44) | |
| 15 | CGACCCTTGGCAGTCTTCAAAAGT | TFEDCQGS | reverse |
| | (SEQ ID NO:45) RT-PCR | (SEQ ID NO:46) | |
| 16 | GACAGATTCTGCCCCGCACCCTTT | KGAGQNLS | reverse |
| | (SEQ ID NO:47) NESTED RT-PCR | (SEQ ID NO:48) | |
| 17 | TCCGGCATCCCCTCCYTNCARGTNGT | SGIPSLQVVDEG | forward |
| | (SEQ ID NO:49) RT-PCR | (SEQ ID NO:50) | |
| 18 | TATCACCAGGTTCCGGAGCGATGA | SSLRNLVI | reverse |
| | (SEQ ID NO:51) RT-PCR | (SEQ ID NO:52) | |
| 19 | CTCAGACCACACGACCTCGAAGAT | IFEVVWSE | reverse |
| | (SEQ ID NO:53) NESTED RT-PCR | (SEQ ID NO:54) | |
| 20 | AAACTCACGTTGTCCGCGTTACAC | CNADNVSL | reverse |
| | (SEQ ID NO:55) 5′-RACE | (SEQ ID NO:56) | |
| 21 | GGCCGATGATAGCGAAGATGACTA | IVIFAIIG | reverse |
| | (SEQ ID NO:57) NESTED 5′-RACE | (SEQ ID NO:58) | |
| 22 | CCTCGGTCTTTTCCAGATTCTGTG | AQNLEKTE | reverse |
| | (SEQ ID NO:59) 5′-RACE | (SEQ ID NO:60) | |
| 23 | GGTCTTATCGCCGTTAGGCAAATG | HLPNGDKT | reverse |
| | (SEQ ID NO:61) NESTED 5′-RACE | (SEQ ID NO:62) | |
| 24 | GCTGATGGCGATGAATGAACACTG | | forward |
| | (SEQ ID NO:63) 5′ RACE OUTER PRIMER | | |
| | FROM AMBION | | |
| 25 | CGCGGATCCGAACACTGCGTTTGCTGGCTTTGATG | | forward |
| | (SEQ ID NO:64) 5′ RACE INNER PRIMER | | |
| | FROM AMBION | | |

The degenerate primers among the above oligonucleotides incorporate a statistical mix of monomers at the positions labeled N (A, G, C or T), H (A, C or T), S (C or G), Y (C or T), W (A or T), D (A, G or T) or R (A or G) [in accordance with IUPAC convention].
Heliothis virescens α1 Subunit Sequence Amplifications

All amplification descriptions for the Heliothis virescens α1 subunit designate sequence positions set forth in SEQ ID NO: 1.

In a RT-PCR reaction, Primers 1 and 2 were used to amplify a fragment from nucleotides 3674 to 4113 Heliothis virescens sequence, which was cloned and sequenced.

The Heliothis sequence was extended upstream by PCR using non-normalized TBW cDNA library plasmid DNA (example 1) with primers 3 and 5, followed by nested PCR with primers 4 and 5. The resulting PCR products generated sequence information from nucleotides 2621 to 3770, which were cloned and sequenced. Primer 5 is SP6 from the library vector sequence.

The Heliothis sequence was extended downstream by PCR using non-normalized TBW cDNA library plasmid DNA (Example 1) with primers 6 and 8, followed by nested PCR with primers 7 and 8. The resulting PCR products generated sequence information from nucleotides 4029 to 5047, which were cloned and sequenced. Primer 8 is T7 from the library vector sequence.

In an attempt for retrieving 3′-end cDNA by 3′-RACE with primers 9 and 11, followed by nested PCR with primers 10 and 12, surprisingly the resulting PCR products generated sequence information from nucleotides 1589 to 2149 instead of 3′-end cDNA sequence. The PCR products were cloned and sequenced. Subsequently, the Heliothis sequence from nucleotides 2149 to 2621 was generated by reverse transcription with random hexamers from a SMART™ RACE cDNA Amplification Kit (BD Biosciences Clontech), followed by touchdown PCR with primers 13 and 15. The resulting cDNA was then amplified with the nested primers 14 and 16 to generate an amplimer from nucleotide 2056 to 2685 which were cloned and sequenced.

The Heliothis sequence was further extended upstream by reverse transcription with random hexamers from a SMART™ RACE cDNA Amplification Kit followed by touchdown PCR with primers 17 and 18. The resulting cDNA was then amplified with the nested primers 17 and 19. The resulting PCR products generated sequence information from nucleotides nucleotide 524 to 1645, which were cloned and sequenced.

The Heliothis sequence was further extended upstream by reverse transcription with random hexamers from a SMART™ RACE cDNA Amplification Kit, followed by touchdown PCR with primers 11 and 20. The cDNA generated from this reaction was then amplified with the nested primers 12 and 21. The resulting PCR products generated sequence information from nucleotides 181 to 613, which were cloned and sequenced.

The 5′-end of Heliothis sequence was further retrieved by reverse transcription with random hexamers from a FirstChoice™ RLM-RACE kit (Ambion), followed by touchdown PCR with primers 22 and 24. The cDNA generated from this reaction was then amplified with the nested primers 23 and 25. The resulting PCR products generated sequence information from nucleotides 1 to 252, which were cloned and sequenced.

Construction of Full-Length Chimera α1 Subunit

The full-length clone for SEQ ID NO: 1 was constructed by joining the PCR fragments above via restriction sites in overlapping DNA segments. In order to add a translation stop codon, the ligated clone was digested with NotI and then fused to the 3′-end of aphid α1 gene with stop codon from nucleotides 1 to 429 listed in SEQ ID NO: 3 derived from cotton aphid library (Example 1) to make the full-length chimeric clone for expression. The chimeric DNA and protein sequences are shown as SEQ ID NO: 5 and 6. The full-length chimeric α1 subunit was inserted into expression vector pCDNA3.1 (Invitrogen Corp).

### Example 3

### Tobacco Budworm (“TBW”) Voltage-Gated Calcium Channel β Subunit Sequence Identifications

Materials and Methods.

Same as Example 2.

Primers. The primers utilized were as shown in table III:

TABLE III

| | | | |
|---|---|---|---|
| Primer | Sequence | Translation | Orientation |

| | | | |
|---|---|---|---|
| 1 | CAGGAGATCGAGCGGATCTTYGARYTNGC | QEIERIFEL | forward |
| | (SEQ ID NO:65) RT-PCR | (SEQ ID NO:66) | |
| | | Deg | |
| 2 | GCTGGTTCTCGTCCAGGATNACRTCRAA | FDVILDENQ | reverse |
| | (SEQ ID NO:67) RT-PCR | (SEQ ID NO:68) | |
| | | Deg | |
| 3 | TCGGACTCTCCAATTGGTAG | RTLQLV | forward |
| | (SEQ ID NO:69) | (SEQ ID NO:70) | |
| 4 | CATGTCAGGGGGGCACTGCG | AQCPPDM | reverse |
| | (SEQ ID NO:71) | (SEQ ID NO:72) | |

The degenerate primers among the above oligonucleotides incorporate a statistical mix of monomers at the positions labeled N (A, G, C or T), H (A, C or T), S (C or G), Y (C or T), W (A or T), D (A, G or T) or R (A or G) [in accordance with IUPAC convention].
Heliothis virescens β Subunit Sequence Amplifications

All amplification descriptions for the Heliothis virescens designate sequence positions set forth in SEQ ID NO: 7.

In a RT-PCR reaction, Primers 1 and 2 were used to amplify a fragment from nucleotides 874 to 1140 Heliothis virescens sequence, which was cloned and sequenced.

A biotinylated probe (from nucleotides 900 to 1113) was synthesized from this fragment by a PCR reaction containing primers 3 and 4 and biotin-dNTP mix from RecActive™ Gene Enrichment Kit (Active Motif, Carlsbad, Calif.). The biotinylated probe was then gel purified and used to clone full length TBW β subunit cDNAs from the non-normalized TBW cDNA library (Example 1) by recA-mediated gene selection, as implemented in the RecActive™ Gene Enrichment Kit from Active Motif (Carlsbad, Calif.). Three full-length clones were sequenced and are shown in SEQ ID NO: 7, 8 and 9. Multiple isoforms were observed as shown in SEQ ID NO: 10, 11 and 12 corresponding to translated protein sequence from SEQ ID NO: 7, 8 and 9, respectively. As these cDNAs were initially cloned in expression vector pCMV-SPORT6.1 (Invitrogen Corp.), no additional subcloning into an expression vector was required.

### Example 4

### Cotton Aphid Voltage-Gated Calcium Channel α2δ Subunit Sequence Identifications

TABLE IV

Oligonucleotides used in isolation of
cotton aphid α2δ subunit cDNAs

| | |
|---|---|
| Name | Sequence |

| | |
|---|---|
| Aph040984U531 | TGGGAAGTTCTTCGGTAAAGTA |
| | (SEQ ID NO:73) |
| Aph040984L959 | GGCCTGGCACACATAGATT |
| | (SEQ ID NO:74) |
| Aph040984U451 | CGGGATTGCTACATACTTGAC |
| | (SEQ ID NO:75) |
| Aph040984L1104 | TTTGTGACAACCCAACGAATTA |
| | (SEQ ID NO:76) |

Cotton aphid calcium channel α2δ sequences were identified by performing a TBLASTN search of the cotton aphid contig database (Example 1) with protein sequence CG12295-PB as the query. CG12295-PB is the predicted translation product of gene CG12295, which is one of three calcium channel α2δ genes identified in the Drosophila melanogaster genome. Reciprocal BLASTX searching of version 3 of the Drosophila protein database with each of the cotton aphid α2δ contigs resulted in the identification of one contig (CAg12001, 1489 bp) that clearly belongs to the cotton aphid ortholog of CG12295.

Full-length cDNAs corresponding to contig CAg12001 were isolated from the cotton aphid cDNA library by RecA-mediated gene enrichment using the RecActive™ kit and protocol from Active Motif (Carlsbad, Calif.). Briefly, plasmid DNA was prepared from an aliquot of the cotton aphid cDNA library comprising approximately 10⁶ primary transformants. Representational biases were minimized by growing the cells for 16 h at 30° C. in 1 L Terrific Broth supplemented with 50 ug/ml carbenicillin. A template for probe synthesis was synthesized from the library DNA by PCR using CAg12001-specific primers Aph040984U531 and Aph040984L959 (Table IV), which were designed with Oligo 6 primer analysis software (Molecular Biology Insights, Inc., Cascade, Colo.). The PCR reaction contained 100 ng library DNA, 5 pmol primers Aph040984U531 and Aph040984L959, 0.3 ul 10 mM dNTP mix (10 mM each of dATP, dGTP, dCTP and TTP), 1.5 ul 10× Titanium™ Taq buffer, and 0.3 ul Titanium™ Taq DNA polymerase (BD Biosciences Clontech, Palo Alto, Calif.) in a final volume of 15 ul. The reaction was heated to 94° C. for 2 minutes and then subjected to 35 cycles of DNA synthesis, each cycle consisting of heat denaturation at 94° C. for 30 seconds, primer annealing at 55° C. for 30 seconds, and primer extension at 72° C. for 1.5 minutes. Reaction products were separated on a low melting temperature agarose gel (SeaPlaque® GTG® agarose, BioWhittaker Molecular Applications, Rockland, Me.) and the expected 450 bp band was extracted from the gel and quantified by fluorometric staining with PicoGreen® dsDNA quantitation reagent (Molecular Probes, Eugene, Oreg.). The purified fragment was used as a template for the synthesis of a biotinylated DNA in a 50 ul PCR reaction containing 100 pg template fragment, 10 pmol primers Aph040984U531 and Aph040984L959, 7 ul biotin-dNTP mix (Active Motif), 5 ul 10× Titanium™ Taq buffer and 1 ul Titanium™ Taq DNA polymerase. The biotinylated probe was purified on a 1% low melting temperature agarose gel and quantified by fluorometric staining with PicoGreen®. Fifty nanograms of the probe fragment were then used to select α2δ clones from 5 ug of cotton aphid library DNA, using the protocol provided with the RecActive™ kit. Approximately 10⁵ colonies from the first round selection were pooled and plasmid DNA was prepared. Quantitative PCR analysis of the pooled DNA indicated that it was enriched approximately 60-fold relative to the starting library for the targeted α2δ sequence. To obtain sufficient enrichment for single colony screening 3 ug of the pooled first round plasmid DNA were reselected with 50 ng of the biotinylated probe. After transforming E. coli DH10B cells with DNA from the second round of selection, individual colonies were screened by PCR using CAg12001-specific primers that flank the region used for biotinylated probe synthesis (primers Aph040984U451 and Aph040984L1104, Table IV). After two rounds of selection, 34 of 36 colonies screened were positive for CAg12001 sequences. Plasmid DNAs were prepared from each positive colony and the cDNA inserts were sized by digestion with Hind III and Kpn I. The cDNA insert from one clone (RA1.11) was sequenced in its entirety. RA1.11 has a 4383 bp insert (SEQ ID NO. 13) that contains a 1245 amino acid open reading frame (SEQ ID NO. 14) which shares 41% amino acid sequence identity over 1239 residues with CG12295-PB. Restriction enzyme analysis and partial sequencing of 15 additional full-length clones demonstrated a complex pattern of alternative splicing involving at least two different regions of the cDNA. One of the alternative splicing patterns involves the differential presence of an 11 amino acid segment represented by residues 805-815 in SEQ ID NO. 14. This segment is not present in the Drosophila CG12295-PB α2δ protein sequence, but is located at the same position and has partial sequence homology to the 11 amino acid sequence that is differentially present in the TBW α2δ protein sequences represented by SEQ ID NOs. 16 and 18 (cf. Example 5).

### Example 5

### Tobacco Budworm (“TBW”) Voltage-Gated Calcium Channel α2δ Subunit Sequence Identifications

TABLE V

Oligonucleotides used in isolation of
tobacco budworm α2δ subunit cDNAs

| | |
|---|---|
| Name | Sequence |

| | |
|---|---|
| HvCaA2D1F | GTGCAGACCTGGGCNGMNAARYT |
| | (SEQ ID NO:77) |
| HVCaA2D1R | GCCGACGATCATGATGGCYTGRTTRCA |
| | (SEQ ID NO:78) |
| HvCaA2DU388 | GCGCCATGATGGAACTTAAGA |
| | (SEQ ID NO:79) |
| HvCaA2DL775 | TGCCGAAGTACTGCCACGACA |
| | (SEQ ID NO:80) |

A fragment derived from the 5′ end of the TBW α2δ CG12295 ortholog was initially synthesized from the TBW head cDNA library (Example 1) by PCR using consensus-degenerate hybrid oligonucleotide primers (CODEHOP primers) designed from blocks of protein sequence conserved in three insect α2δ orthologs: CG12295-PB from Drosophila melanogaster, ENSANGP00000021218 from Anopheles gambiae and RA1.11 from Aphis gossypii (SEQ ID NO. 14, Example 4). A database of protein blocks conserved among these three sequences was constructed with Block Maker (Henikoff. and Henikoff, Nucleic Acids Research, Vol. 19, pp. 6565-6572, (1991)) and used as input for the CODEHOP primer design program (Rose, et al., Nucleic Acids Research, Vol. 26, pp. 1628-1635 (1998)). The degenerate forward primer HvCaA2D1F (Table V) was derived from the amino acid sequence VQTWAEKL SEQ ID NO:81 in the 5′ most conserved protein block, while the degenerate reverse primer HvCaA2D1R (Table V) was derived from the complementary strand of the conserved sequence CNQAIMIVS SEQ ID NO:82 located roughly a third of the way into the protein from the N-terminus. Touchdown PCR was performed with 100 ng of plasmid DNA isolated from the TBW head cDNA library (Example 1) and a 900 bp fragment was isolated by agarose gel electrophoresis and cloned into pBluescript SK (−) (Stratagene). DNA sequence analysis confirmed the band was derived from the TBW ortholog of Drosophila gene CG12295. Two non-degenerate gene-specific primers (HvCaA2DU388 and HvCaA2DL775, Table V) were designed from the sequence of the 900 bp fragment and used for the preparation of a biotinylated probe, as described in Example 4, except that the annealing temperature for this PCR reaction was 62° C. rather than 55° C. The biotinylated probe was used for two rounds of recA-mediated gene enrichment (cf. Example 4), starting with 5 ug of plasmid DNA from the TBW nerve cord library (Example 1). Three of 24 colonies tested by PCR after the second round of selection were positive for TBW α2δ sequences. All three plasmids contained inserts of ˜5 kb. Two of the clones (clone 2 and clone 7) were sequenced in their entirety. Clone 2 (5028 bp, SEQ ID NO. 15) contains an open reading frame of 1271 amino acids (SEQ ID NO. 16), while clone 7 (4892 bp, SEQ ID NO. 17) contains an open reading frame protein of 1258 amino acids (SEQ ID NO. 18). The 5′ UTR segment present in clone 2 is 98 bp longer than that found in clone 7, but the two 5′ UTR sequences are otherwise identical. However, the two clones appear to derive from different alleles in the outbred TBW population used for library construction, since there are 69 single nucleotide polymorphisms in the coding regions of the two cDNAs, and a number of small insertions/deletions in the 3′ UTR. In addition clone 2 contains two small insertions in the coding region relative to clone 7: a 2 amino acid insertion at residues 490-410 (VK) and an 11 amino acid insertion at residues 842-852 (PLTKVIGLLPR SEQ ID NO:83). Although the coding regions contain a large number of single nucleotide polymorphisms, only two of these result in a difference in protein sequence, and both changes are conservative amino acid substitutions.

### Example 6

### Functional Expression of Tobacco Budworm (“TBW”) Voltage-Gated Calcium Channel in Xenopus Oocytes

Materials and Methods.

Generation of capped mRNA with poly (A) tail: Chimera α1 subunit in pCDNA3.1 was linearized by NsiI; TBW β subunit in pCMV-SPORT6.11 was linearized by DrdI; Aphid α2δ in pCMV-SPORT6.1 was linearized by NotI; and TBW □2□ in pGreen1S (+) Lox2 (Xba) was linearized by NheI. The linearized DNA was then purified by phenol-chloroform extraction and ethanol precipitation. The DNA was used for making capped mRNA following the manual of mMESSAGE mMACHINE™ high yield capped RNA transcription kit from Ambion. After transcription reaction, the samples were treated with DNase I to remove the template DNA and then a poly(A) tail was added to the RNA with poly(A) polymerase from Ambion. The capped mRNA with poly(A) tail was phenol-choloform extracted and ethanol precipitated. The concentration of capped mRNA with poly(A) tail was measured by UV spectrometry. The mRNA solution was stored at −20° C. for future use.

Oocytes expression and patch claming: Methods used in the Xenopus oocyte gene expression system are well established in the field (Methods in Enzymology, Vol. 207, eds: Rudy, B. and Iverson, L. E., section II, A. Expression of Ion channels in Xenopus oocytes, pp. 225-390). The technique is briefly described below. Unfertilized oocytes were surgically removed from Xenopus laevis frogs (NASCO, Fort Atkinson, Wis.) anesthetized by submersion in 0.15% Tricaine (3-amino benzoic acid ethyl ester). The eggs were rinsed in a calcium free saline solution (OR2: NaCl 82.5 mM, KCl 2 mM, MgCl2 1 mM, HEPES 5 mM, pH 7.4) and subsequently incubated in OR2 containing 4 mg/mL collagenase for 2 hours at 17° C. on an orbital shaker. Oocytes were then mechanically defolliculated using forceps and transferred to normal calcium containing saline (ND96: NaCl 96 mM, KCl 2 mM, MgCl2 1 mM, CaCl2 1.8 mM, HEPES 5 mM, pH 7.4). RNA transcripts from the desired gene or genes were then injected into each oocyte with a piston driven microinjector (Drummond Nanoject, Drummond Scientific Company, Broomall, Pa.). In a typical experiment 4-6 ng of each subunit's RNA was delivered in 23 nL of water. Injected oocytes were incubated on an orbital shaker at 17° C. and examined for functional expression of the injected channel in 2 to 4 days.

Oocytes were tested for functional expression of ion channels using a 2-electrode voltage clamp (AxoClamp 2B, Axon Instruments, Union City, Calif.). Standard voltage-clamp current passing and recording techniques were used. Recording electrodes were fabricated using a Narishige pipette puller (Model PP830, Narishige International USA, Long Island, N.Y.) and were adjusted to have an input resistance of between 0.8 and 3 megaohms when filled with 4 M potassium acetate. Data were gathered using a Pentium® class computer with an analog to digital interface (Digidata 1322, Axon Instruments) running pClamp8 software (Axon Instruments). Recordings to assess the performance of calcium channels were made in NS saline (Ba(OH)₂ 40 mM, Na-D-gluconate 50 mM, KOH 1 mM, EDTA 0.1 mM, HEPES 10 mM, niflumic acid 0.1 mM, pH 7.4) that was formulated to minimize endogenous calcium activated currents.

Results: Various combinations of 3 invertebrate calcium channel subunits were tested. The α1 subunit shown on SEQ ID NO: 6, β-2 subunit shown on SEQ ID NO:10, the α2δ-2 subunit shown on SEQ ID NO: 18 were derived from Heliothis virescens. An α2δ subunit shown on SEQ ID NO: 14 from cotton aphid was also tested to determine whether that subunit could substitute for the native H. virescens α2δ.

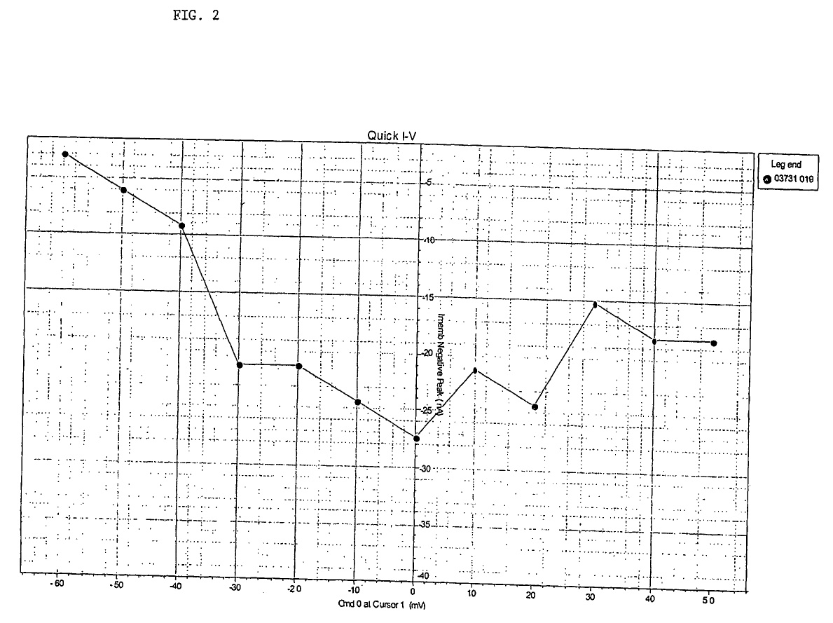

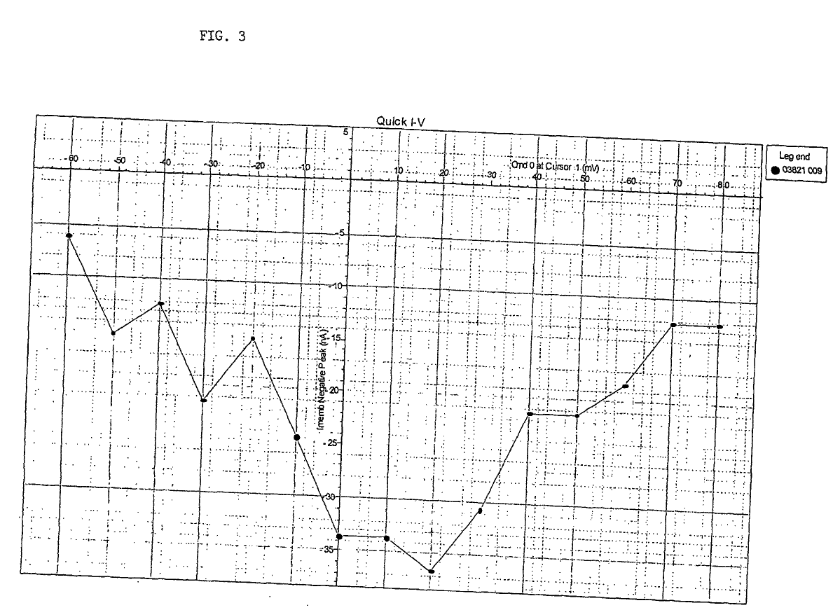

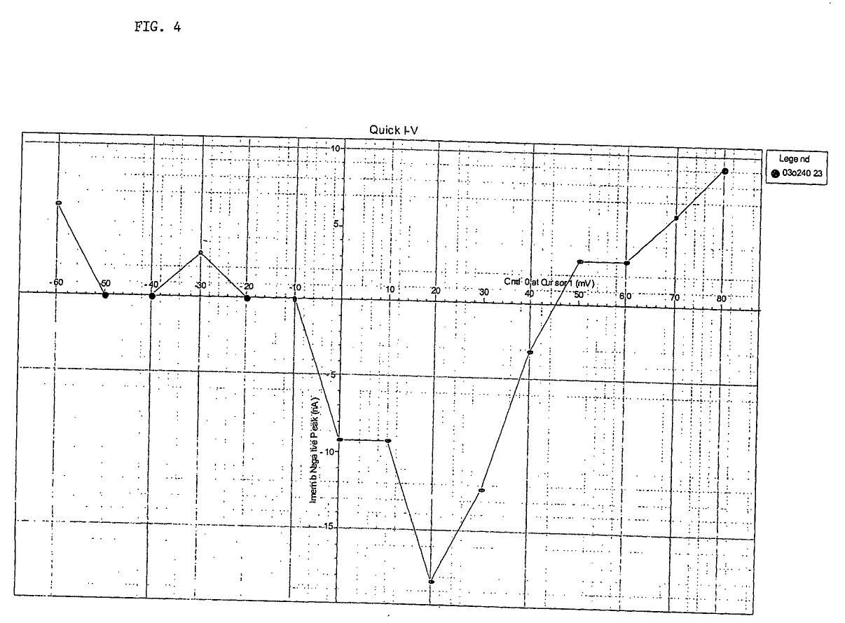

RNA for each subunit was injected at a dose of 6 ng/egg. The α1 subunit alone failed to yield any voltage gated currents despite the fact that control voltage-gated channels injected into eggs from each batch of harvested eggs produced robust currents (data not shown). Co-expression of the α1 and β-2 subunit produced modest currents typically in the range of 10 to 20 nA as shown in FIG. 2. However, current expression was unstable and inconsistent from egg to egg. The addition of aphid α2δ subunits to the α1 and β-2 subunit produced small but clearly defined voltage-sensitive N-type calcium currents (FIG. 3). Replacement of the aphid α2δ with the TBW α2δ-2 subunit also resulted in the formation of clearly defined voltage-sensitive N-type calcium currents (FIG. 4), but the amplitude of the observed peak current increased to amplitudes as great as 40 nA.

We conclude from the data gathered in the fashion described above, that the currents generated in response to voltage steps while in voltage-clamp were similar to N-type calcium channels observed in whole cell patch-clamp recordings made from neurons grown in culture (Hayashi and Levine, Exp. Biol. Vol. 171, pp. 15-42 (1992)). Both the cloned channel under study and those observed in cultured neurons exhibit an activation point of roughly −30 mV and an apparent reversal potential in the range of +50 mV. While the currents observed in oocytes are diminutive, their kinetics are reminiscent of the slowly inactivating currents observed in cultured insect neurons. We further conclude that the chimeric α1 subunit shown in SEQ ID:6 is capable of forming functional voltage-sensitive ion channels reminiscent of N-type calcium channels in the presence of the appropriate insect β and α2δ calcium channel subunits.

1. A substantially pure protein having the amino acid sequence selected from the group consisting of: SEQ ID NO: 2, SEQ ID NO: 6, SEQ ID NO: 8, SEQ ID NO: 10, SEQ ID NO: 12, SEQ ID NO 14, SEQ ID NO: 16, SEQ ID NO: 18, a mutant thereof, and a fragment thereof.

2. The protein of claim 1 wherein said protein has the amino acid sequence of SEQ ID NO: 2, SEQ ID NO: 6, SEQ ID NO: 8, SEQ ID NO: 10, SEQ ID NO: 12, SEQ ID NO 14, SEQ ID NO: 16 or SEQ ID NO: 18.

3. An isolated nucleic acid molecule that comprises a nucleic acid sequence that encodes the protein of claim 1.

4. A recombinant expression vector comprising the nucleic acid molecule of claim 3.

5. A host cell comprising the recombinant expression vector of claim 4.

6. An isolated nucleic acid molecule that comprises a nucleic acid sequence that encodes the protein of claim 2.

7. A recombinant expression vector comprising the nucleic acid molecule of claim 6.

8. A host cell comprising the recombinant expression vector of claim 7.

9. An isolated nucleic acid molecule consisting of: SEQ ID NO: 1, a fragment thereof having at least 10 nucleotides, SEQ ID NO: 5, a fragment thereof having at least 10 nucleotides, nucleotides, SEQ ID NO: 7, a fragment thereof having at least 10 nucleotides, nucleotides, SEQ ID NO: 9, a fragment thereof having at least 10 nucleotides, nucleotides, SEQ ID NO: 11, a fragment thereof having at least 10 nucleotides, SEQ ID NO 13, a fragment thereof having at least 10 nucleotides, SEQ ID NO: 15, a fragment thereof having at least 10 nucleotides, nucleotides, SEQ ID NO: 17, and a fragment thereof having at least 10 nucleotides, wherein a fragment of SEQ ID NO: 5 comprises at least 5 nucleotides of SEQ ID NO: 1.

10. The nucleic acid molecule of claim 9 consisting of: SEQ ID NO: 1, SEQ ID NO: 5, SEQ ID NO: 7, SEQ ID NO: 9, SEQ ID NO: 11, SEQ ID NO 13, SEQ ID NO: 15 and SEQ ID NO: 17.

11. A recombinant expression vector comprising the nucleic acid molecule of claim 10.

12. A host cell comprising the recombinant expression vector of claim 11.

13. The nucleic acid molecule of claim 9 consisting of: a fragment of SEQ ID NO: 1 having at least 10 nucleotides, a fragment of SEQ ID NO: 5 having at least 10 nucleotides, a fragment of SEQ ID NO: 7 having at least 10 nucleotides, a fragment of SEQ ID NO: 9 having at least 10 nucleotides, a fragment of SEQ ID NO: 11 having at least 10 nucleotides, a fragment of SEQ ID NO: 13 having at least 10 nucleotides, a fragment of SEQ ID NO: 15 having at least 10 nucleotides, and a fragment of SEQ ID NO: 17 having at least 10 nucleotides, wherein a fragment of SEQ ID NO: 5 comprises at least 5 nucleotides of SEQ ID NO: 1.

14. The nucleic acid molecule of claim 9 consisting of a fragment of SEQ ID NO: 1 having 12-150 nucleotides, a fragment of SEQ ID NO: 5 having 12-150 nucleotides, a fragment of SEQ ID NO: 7 having 12-150 nucleotides, a fragment of SEQ ID NO: 9 having 12-150 nucleotides, a fragment of SEQ ID NO: 11 having 12-150 nucleotides, a fragment of SEQ ID NO: 13 having 12-150 nucleotides, a fragment of SEQ ID NO: 15 having 12-150 nucleotides, and a fragment of SEQ ID NO: 17 having 12-150 nucleotides, wherein a fragment of SEQ ID NO: 5 comprises at least 5 nucleotides of SEQ ID NO: 1.

15. The nucleic acid molecule of claim 9 consisting of a fragment of SEQ ID NO: 1 having 15-50 nucleotides, a fragment of SEQ ID NO: 5 having 15-50 nucleotides, a fragment of SEQ ID NO: 7 having 15-50 nucleotides, a fragment of SEQ ID NO: 9 having 15-50 nucleotides, a fragment of SEQ ID NO: 11 having 15-50 nucleotides, a fragment of SEQ ID NO: 13 having 15-50 nucleotides, a fragment of SEQ ID NO: 15 having 15-50 nucleotides, and a fragment of SEQ ID NO: 17 having 15-50 nucleotides, wherein a fragment of SEQ ID NO: 5 comprises at least 5 nucleotides of SEQ ID NO:1.

16. An isolated antibody which binds to an epitope on SEQ ID NO: 2, SEQ ID NO: 6, SEQ ID NO: 8, SEQ ID NO: 10, SEQ ID NO: 12, SEQ ID NO 14, SEQ ID NO: 16 or SEQ ID NO: 18, wherein an antibody that binds to SEQ ID NO: 6 does not cross react with SEQ ID NO: 4.

17. The antibody of claim 16 wherein said antibody is a monoclonal antibody.

18. A method of identifying a modulator of a lepidoptera calcium channel protein activity comprising the steps of:
performing a test assay by contacting a test cell that comprises a functional calcium channel, wherein the test cell comprises at least one of a recombinant expression vector that comprises a nucleotide sequence that encodes a chimeric TBW voltage-gated calcium channel α1 subunit, a recombinant expression vector that comprises a nucleotide sequence that encodes a TBW voltage-gated calcium channel β subunit and a recombinant expression vector that comprises a nucleotide sequence that encodes a TBW voltage-gated calcium channel α2δ subunit wherein said test cell expresses a functional calcium channel, with a solution containing calcium in the presence of a test compound, and detecting the amount of intracellular calcium in said test cell;
performing a negative control assay by contacting a negative control cell that comprises a functional calcium channel, wherein the test cell comprises at least one of a recombinant expression vector that comprises a nucleotide sequence that encodes a chimeric TBW voltage-gated calcium channel α1 subunit, a recombinant expression vector that comprises a nucleotide sequence that encodes a TBW voltage-gated calcium channel β subunit and a recombinant expression vector that comprises a nucleotide sequence that encodes a TBW voltage-gated calcium channel α2δ subunit and wherein said negative control cell expresses a functional calcium channel, with a solution containing calcium in the absence of said test compound, and detecting the amount of intracellular calcium in said negative control cell;
comparing the amount of intracellular calcium in said test cell to the amount of intracellular calcium in said negative control cell
wherein a change in the amount of calcium in said test cell compared to the amount of intracellular calcium in said negative control cell indicates the test compound is a modulator of calcium channel activity.

19. The method of claim 18 wherein the test cell is a Xenopus oocyte cell, a CHO cell or an HEK cell.

20. The method of claim 18 wherein the intracellular calcium is detected by using an assay in which fluorescence generated by dye inside of said cell interacting with intracellular calcium is measured.

21. The method of claim 18 further comprising performing a positive control assay by contacting a positive control cell which comprises a functional calcium channel, with a solution containing calcium in the absence of said test compound and in the presence of a calcium channel agonist, and detecting the amount of intracellular calcium in said positive control cell.

22. The method of claim 21 wherein the calcium channel is a lepidoptera calcium channel.

23. The method of claim 18 further comprising the step of
performing a second-type negative control assay by contacting a negative control cell that does not express a functional calcium channel with a solution containing calcium in the absence of said test compound, and detecting the amount of calcium taken up by said calcium channel negative control cell; and/or
performing a third-type negative control assay by contacting a negative control cell that does not express a functional calcium channel with a solution containing calcium in the absence of said test compound and in the presence of a calcium channel agonist, and detecting the amount of calcium taken up by said calcium channel negative control cell.

24. The method of claim 23 wherein the calcium channel is a lepidoptera calcium channel.

25. The method of claim 18 wherein said functional calcium channel comprises at least one of: a chimeric TBW voltage-gated calcium channel α1 subunit, a TBW voltage-gated calcium channel β subunit and a TBW voltage-gated calcium channel α2δ subunit, wherein the chimeric TBW voltage-gated calcium channel α1 subunit comprises SEQ ID NO: 2, a mutant thereof, and a fragment thereof; the TBW voltage-gated calcium channel β subunit comprises SEQ ID NO: 8, SEQ ID NO: 10, SEQ ID NO: 12, a mutant thereof, and a fragment thereof and the TBW voltage-gated calcium channel α2δ subunit comprises SEQ ID NO: 16, SEQ ID NO: 18, a mutant thereof, and a fragment thereof.

26. The method of claim 25 wherein the test cell is a Xenopus oocyte cell, a CHO cell or HEK cell.

27. The method of claim 25 wherein the intracellular calcium is detected by using an assay in which fluorescence generated by dye inside of said cell interacting with intracellular calcium is measured.

28. The method of claim 25 wherein said functional calcium channel comprises a chimeric TBW voltage-gated calcium channel α1 subunit that comprises SEQ ID NO: 2; a TBW voltage-gated calcium channel β subunit that comprises SEQ ID NO: 8, SEQ ID NO: 10 or SEQ ID NO: 12 and a TBW voltage-gated calcium channel α2δ subunit comprises SEQ ID NO: 16 or SEQ ID NO: 18.

29. The method of claims 28 wherein that chimeric TBW voltage-gated calcium channel α1 subunit comprises SEQ ID NO: 6.

30. The method of claim 28 wherein the nucleic acid sequence that encodes the chimeric TBW voltage-gated calcium channel α1 subunit that comprises SEQ ID NO: 1; the nucleic acid sequence that encodes the TBW voltage-gated calcium channel β subunit comprises SEQ ID NO: 7, SEQ ID NO: 9 or SEQ ID NO: 11; and the nucleic acid sequence that encodes the TBW voltage-gated calcium channel α2δ subunit comprises SEQ ID NO: 15 or SEQ ID NO: 17.

31. The method of claim 30 wherein the nucleic acid sequence that encodes the chimeric TBW voltage-gated calcium channel α1 subunit that comprises SEQ ID NO: 5.

32. A method of identifying an inhibitor of a lepidoptera calcium channel protein activity comprising the steps of:
performing a test assay by contacting a test cell that comprises a functional calcium channel, wherein the test cell comprises at least one of a recombinant expression vector that comprises a nucleotide sequence that encodes a chimeric TBW voltage-gated calcium channel α1 subunit, a recombinant expression vector that comprises a nucleotide sequence that encodes a TBW voltage-gated calcium channel β subunit and a recombinant expression vector that comprises a nucleotide sequence that encodes a TBW voltage-gated calcium channel α2δ subunit and wherein said test cell expresses a functional calcium channel, with a solution containing calcium and a calcium channel agonist in the presence of a test compound, and detecting the amount of intracellular calcium in said test cell;
performing a control assay by contacting a negative control cell that comprises a functional calcium channel, wherein said negative control cell expresses a functional calcium channel, with a solution containing calcium and a calcium channel agonist in the absence of said test compound, and detecting the amount of intracellular calcium in said negative control cell;
comparing the amount of intracellular calcium in said test cell to the amount of intracellular calcium in said control cell
wherein a decrease in the amount of intracellular calcium in said test cell compared to the amount of intracellular calcium in said control cell indicates the test compound is an inhibitor of calcium channel activity.

33. The method of claim 32 wherein the test cell is a Xenopus oocyte, a CHO cell or an HEK cell.

34. The method of claim 32 wherein the intracellular calcium is detected by using an assay in which fluorescence generated by dye inside of said cell interacting with intracellular calcium is measured.

35. The method of claim 38 wherein said functional calcium channel comprises at least one of a chimeric TBW voltage-gated calcium channel α1 subunit that comprises SEQ ID NO: 2, a mutant thereof, and a fragment thereof; a TBW voltage-gated calcium channel β subunit that comprises SEQ ID NO: 8, SEQ ID NO: 10, SEQ ID NO: 12, a mutant thereof, or a fragment thereof and the TBW voltage-gated calcium channel α2δ subunit that comprises SEQ ID NO: 16, SEQ ID NO: 18, a mutant thereof, or a fragment thereof.

36. The method of claim 35 wherein the test cell is a Xenopus oocyte, a CHO cell or an HEK cell.

37. The method of claim 35 wherein the intracellular calcium is detected by using an assay in which fluorescence generated by dye inside of said cell interacting with intracellular calcium is measured.

38. The method of claim 35 wherein said functional calcium channel comprises a chimeric TBW voltage-gated calcium channel α1 subunit that comprises SEQ ID NO: 2; a TBW voltage-gated calcium channel β subunit that comprises SEQ ID NO: 8, SEQ ID NO: 10 or SEQ ID NO: 12; and a TBW voltage-gated calcium channel α2δ subunit comprises SEQ ID NO: 16 or SEQ ID NO: 18.

39. The method of claims 38 wherein that chimeric TBW voltage-gated calcium channel α1 subunit comprises SEQ ID NO: 6.

40. The method of claim 38 wherein the nucleic acid sequence that encodes the chimeric TBW voltage-gated calcium channel α1 subunit that comprises SEQ ID NO: 1; the nucleic acid sequence that encodes the TBW voltage-gated calcium channel β subunit comprises SEQ ID NO: 7, SEQ ID NO: 9 or SEQ ID NO: 11 and the nucleic acid sequence that encodes the TBW voltage-gated calcium channel α2δ subunit comprises SEQ ID NO: 15 or SEQ ID NO: 17.

41. A method of preparing an isolated protein having the amino acid sequence selected from the group consisting of SEQ ID NO: 2, a mutant thereof, and a fragment thereof; SEQ ID NO: 6, a mutant thereof, a fragment thereof, SEQ ID NO: 8, a mutant thereof, a fragment thereof, SEQ ID NO: 10, a mutant thereof, a fragment thereof, SEQ ID NO: 12, a mutant thereof, a fragment thereof, SEQ ID NO: 16, a mutant thereof, a fragment thereof SEQ ID NO: 18, a mutant thereof, and a fragment comprising the step of isolating said protein from a host cell of claim 5.

42. A method of controlling an insect, comprising contacting an insect with a modulator identified by a method according to claim 18.

43. A method of controlling an insect, comprising contacting an insect with an inhibitor identified by a method according to claim 32.


SEQUENCE LISTING (83 sequences)

SEQ ID NO: 1 - DNA, 5047 bp, Heliothis virescens
ggttggcggcagcgcaggaggcggcgggggcgcaggctcgcgcggcgccagctgataccatgggcggcgcgcaccagcccgctactccagggcccagctcactctttatatttgccgacgaaaatcctattcggaggtacacaaagttcatcatcgagtggccgcccttcgagtacgcggtgctgcttaccatcatcgccaactgcgtggtgctggcgctggaggagcatttgcctaacggcgataagaccatcttagcacagaatctggaaaagaccgaggcgtactttttaggaatattttgtgtagaagcctcgttaaaaatcttagccttaggttttgttttacacaggggatcgtatcttaggaacgtttggaacatcatggattttttcgttgtagtaactggtatcatcacgcagctgccgatcgcgccagccgacgtcgacttcaggaccttgcgtgccattagggtgctgaggccccttaaattagtatcgggcgttcctagtctgcaagtggtactgaagtccatcataaaggcgatggcgccgttgctgcagatcggcctcctggtgcttttcgcgatagtcatcttcgctatcatcggcctcgagttctactcaggggcgctgcataagacttgttataatttagaagatattagtgaaatagtaaatgaaggcgatagtgcgacgccgtgtaacgcggacaacgtgagtttagcaccatttggggcaaacgtgtgtgattatgagaagagcacgtgtttagagaaatgggaggggccgaacaggggtattacgtccttcgacaacatcggcttcgctatgctcaccgtcttccagtgcattaccatggagggctggaccgcaatcctctattggacgaatgacgcgctaggtagtgcgttcaactggatttactttgtgcctctcatagtattgggttcattctttatgctcaacttagttctcggtgtccttagcggtgagttcgctaaagaaagagagaaagtagaaaatagacaagaatttcttaaattaagaagacagcagcaactcgagagagaactcaatggttacgttgagtggatttgtaaagcagaggaagtaatattagcagaagaaagaacaacagaagaagaaaaaatgcacataatagaagcacggagaagagcagcggccaaaaagaagttaaaaaaccttggtaaaagtaaaagcacagatacagaagaggaagaacaagatgaagactgcggtgatgacggttttctaaaaagcaaagctcggtcagccgggaggtttgcggacttctggcgggctgagaagaggtttcggttttggatcaggcacacagtgaagacccagtggttctactggttcgtcattgtcttggtgctcttcaacacgatatgcgtcgctgtagagcattatagacaacccaagtggctgacttcgtttttatactatgccgaatttgtgttcctggggttgttcatgatggagatgtgggtgaagatgtatgcgcttgggccgcgaatctacttcgagtcgtccttcaaccggttcgactgcgtggtcatctccggctccatcttcgaggtcgtgtggtctgaggtcaagggtggctccttcggtctatctgtcctgagagctctaagactgttgaggatatttaaggtcaccaagtactggtcatcgctccggaacctggtgatatctctcctcaactcaatgagatccatcatctcgctgctgttcctgctcttcctgttcattctcatcttcgcactgctcggcatgcagctgttcgggggacagttcaacttcgaggacggcacgccgccgaccaacttcaacacctttcctatcgcgttgttaactgtcttccagatcctaacaggtgaagattggaacgaagtgatgtatgacggcatccagtcacagggcggcatccagagaggcatgatctactctctatactttgtcatcctcgtcttatttggcaactacacgctgctgaacgtgttccttgctatcgctgtcgacaacttggctaacgcccaggaattgacggcggcagaagaggaacaagtcgaggaggacaaggagaaacagctccaggaattggagaaagggatgggtgcattacacgcggtggacggcactccaccgggagtagatctaagtccctcttcgccgacgagtaggaagaacaaaaagaaagaagaggccaaaaaagaagatgaagatgaggtaccagatggaccaaaaccaatgctgccatattcgtccatgtttattttgtcacctactaatccaattaggcgaggcgcacactgggttgtaaatttaagatattttgatttttttatcatggtagttatatgtatgagttctgcggctttagcggctgaagaccccgtagtggaagagagtgacaggaacaaaatcctgaactacttcgattacgcgttcacgggcgtgttcaccgtggagatgctgctgaagatagtggacctcggcatcctgttccacccgggcgcctacctgcgcgacctgtggaacatcatggatgccgccgtcgtcatatgcgcccttgtcagcttcggatttgagatcggaggcgtgaaaaagggggcggggcagaatctgtccacaataaaatcgttaagagtgttacgagtgctcagacctttgaaaactataaaacgagttccaaagttaaaagcagtgtttgactgtgttgtgaactctttgaaaaacgtcattaacattctcattgtgtacatattgtttcaattcatattcgctgtaattgcagttcaactttttaatggtaaattttttcactgcaacgatatcagtaagaatacttttgaagactgccaagggtcgtatttcgtgtacgagtcaaacagcttgctgccgaaagtcaaccagcgcacgtggacgacgcaatccttccattacgacaacgtcgccgtggctatgcttacgctgttcgccgtgcagactggggaggggtggccacaagtattacaaaattcaatggccgccacctacgaagacaggggacccatacaaaattttcgaatagaaatgtccatattttatatagtttacttcgtggtgtttcctttcttctttgttaacatattcgtagctctgataattatcacatttcaagagcagggcgaagctgagcttcaggatggtgaaattgataagaatcagaaatcgtgtatagacttcacgatagaagcgcgacctctcgagaggtatatgccaagcaaaagggcgagttttaagtacaaagtgtggagaatagttgtctctacgcccttcgagtacttcatcatgacgctgatcgtcctcaacacattgttgctcatgatgaagtttcacgaggctccaccactactcatggacatattaacattcatgaacctcgtctttacgaccttcttccttctcgagaccgtattgaagctgatcgccttcgggtgtacgaattttttcaaagacccttggaatacattcgattttattacggtcattggaagtattattgacgccctcattatggagtttggcgagaacacattcaacgtcggtttccttcgcctgttccgagccgcgcgactgatcaagctgctccgacagggctacactattcggatactgctctggacattcgtgcagagtttcaaagccttaccctacgtgtgccttctcatcgcgatgctattcttcatctacgccatcatcgggatgcaggtgtttggcaatatagaattaacaccagagtctgacatgaacagacacaacaattttcgaagcttcattcaagcactcatgctactgttcagatgcgcaacgggcgagtcgtggcccaacataatgttggcttgccgcaaacccgccaagtgcgacatagcagctggaaaggcctccaacgaagaatgtggaagtacgctcgcctacgcctacttcgtatcttttatattcttctgttcgtttcttatgttgaatttgttcgttgctgttattatggataactttgactacctaacgagggactcgtccatcctcggcgcacatcatcttgatgaatttgttagaatatgggcagaatatgatccaaacgccacgggtaagatccattatacagaaatgtatgatatgttgaagaatatggatccgcctctggggtttggtaacaaatgtccaaatagactagcatataagaagcttattagaatgaatatgccgctagacgatgaggggaaagttaattttacaacaacactatttgccttaatacgagaaaacttgaacatcaaaatgagatctcccgaggaaatggaccaagcagatgaggaattaagggaaacaataacccacatttggccattacaagcgaagaagatgctcgacctgctggtgcctcgaaacgatgtactcaacgctggaaaactgaccgtcgggaagatatacgctggacttctaatcctcgagagttggagatctacaaggttcaagcagaatggtgttccggtactggaactacaaggatcacaccacgcctcaatggagtcgttggacgagggacggttacaagctcctcacacgtaccagaatggacaccaccatgggagatcatccagtttaagacgaacgcccagtccaagaagacgaggccactacggaggttatcatcacgatatcggattctcagacaccgtcagcaacgtcgtcgagatagtaaagcatgaacaccagagacacgggcgaacgcacagagcgccccactactaccacccacatgtttgggccccgataggtgagcgggagcgggaccgcgagtggcgggagtggcgcgaccgctcctgggagcgcgagggcgcgcgccgcggccgcggccgccagttgccccccacgcccaccaagccgtccacgctacaggtcaagcagcagccacccatcaccagaatcagccacagcccctcacacttatcgctaaggcacaccgtcagagaacc

SEQ ID NO: 2 - protein, 1662 aa, Heliothis virescens
MGGAHQPATPGPSSLFIFADENPIRRYTKFIIEWPPFEYAVLLTIIANCVVLALEEHLPNGDKTILAQNLEKTEAYFLGIFCVEASLKILALGFVLHRGSYLRNVWNIMDFFVVVTGIITQLPIAPADVDFRTLRAIRVLRPLKLVSGVPSLQVVLKSIIKAMAPLLQIGLLVLFAIVIFAIIGLEFYSGALHKTCYNLEDISEIVNEGDSATPCNADNVSLAPFGANVCDYEKSTCLEKWEGPNRGITSFDNIGFAMLTVFQCITMEGWTAILYWTNDALGSAFNWIYFVPLIVLGSFFMLNLVLGVLSGEFAKEREKVENRQEFLKLRRQQQLERELNGYVEWICKAEEVILAEERTTEEEKMHIIEARRRAAAKKKLKNLGKSKSTDTEEEEQDEDCGDDGFLKSKARSAGRFADFWRAEKRFRFWIRHTVKTQWFYWFVIVLVLFNTICVAVEHYRQPKWLTSFLYYAEFVFLGLFMMEMWVKMYALGPRIYFESSFNRFDCVVISGSIFEVVWSEVKGGSFGLSVLRALRLLRIFKVTKYWSSLRNLVISLLNSMRSIISLLFLLFLFILIFALLGMQLFGGQFNFEDGTPPTNFNTFPIALLTVFQILTGEDWNEVMYDGIQSQGGIQRGMIYSLYFVILVLFGNYTLLNVFLAIAVDNLANAQELTAAEEEQVEEDKEKQLQELEKGMGALHAVDGTPPGVDLSPSSPTSRKNKKKEEAKKEDEDEVPDGPKPMLPYSSMFILSPTNPIRRGAHWVVNLRYFDFFIMVVICMSSAALAAEDPVVEESDRNKILNYFDYAFTGVFTVEMLLKIVDLGILFHPGAYLRDLWNIMDAAVVICALVSFGFEIGGVKKGAGQNLSTIKSLRVLRVLRPLKTIKRVPKLKAVFDCVVNSLKNVINILIVYILFQFIFAVIAVQLFNGKFFHCNDISKNTFEDCQGSYFVYESNSLLPKVNQRTWTTQSFHYDNVAVAMLTLFAVQTGEGWPQVLQNSMAATYEDRGPIQNFRIEMSIFYIVYFVVFPFFFVNIFVALIIITFQEQGEAELQDGEIDKNQKSCIDFTIEARPLERYMPSKRASFKYKVWRIVVSTPFEYFIMTLIVLNTLLLMMKFHEAPPLLMDILTFMNLVFTTFFLLETVLKLIAFGCTNFFKDPWNTFDFITVIGSIIDALIMEFGENTFNVGFLRLFRAARLIKLLRQGYTIRILLWTFVQSFKALPYVCLLIAMLFFIYAIIGMQVFGNIELTPESDMNRHNNFRSFIQALMLLFRCATGESWPNIMLACRKPAKCDIAAGKASNEECGSTLAYAYFVSFIFFCSFLMLNLFVAVIMDNFDYLTRDSSILGAHHLDEFVRIWAEYDPNATGKIHYTEMYDMLKNMDPPLGFGNKCPNRLAYKKLIRMNMPLDDEGKVNFTTTLFALIRENLNIKMRSPEEMDQADEELRETITHIWPLQAKKMLDLLVPRNDVLNAGKLTVGKIYAGLLILESWRSTRFKQNGVPVLELQGSHHASMESLDEGRLQAPHTYQNGHHHGRSSSLRRTPSPRRRGHYGGYHHDIGFSDTVSNVVEIVKHEHQRHGRTHRAPHYYHPHVWAPIGERERDREWREWRDRSWEREGARRGRGRQLPPTPTKPSTLQVKQQPPITRISHSPSHLSLRHTVRE

SEQ ID NO: 3 - DNA, 429 bp, Aphis gossypii
aggcgccggttgccgcccacgcccaacaaaccgtcgtcgctgcagctccggccgccgcaccactcggcctcgataaactttccgaagctgaacgcgtcgccgacgcgcggccaaccggcctcgctgcccaaaccgccgcccgtcaacatgtcgtccacgtcgacggccgcggccgtggcccggcagagcatggccatgaccgccatgccgccgctcagcttcgagcaggccatgtcgatcggccgcagtggccggctactgccgtcgccggtgcgcaacgggtacgcgtcgtcgtcgtccaagcagcagcagcagcagcagcagccgtcgtcgtcgtcgtcggtgtccgcgatcaggcggcaaaagagcggcggcggtggccgggtgagcagccggcacagcgattcggacgaggacgactggtgctaa

SEQ ID NO: 4 - protein, 142 aa, Aphis gossypii
RRRLPPTPNKPSSLQLRPPHHSASINFPKLNASPTRGQPASLPKPPPVNMSSTSTAAAVARQSMAMTAMPPLSFEQAMSIGRSGRLLPSPVRNGYASSSSKQQQQQQQPSSSSSVSAIRRQKSGGGGRVSSRHSDSDEDDWC

SEQ ID NO: 5 - DNA, 5306 bp, Heliothis virescens and Aphis gossypii
cagctgataccatgggcggcgcgcaccagcccgctactccagggcccagctcactctttatatttgccgacgaaaatcctattcggaggtacacaaagttcatcatcgagtggccgcccttcgagtacgcggtgctgcttaccatcatcgccaactgcgtggtgctggcgctggaggagcatttgcctaacggcgataagaccatcttagcacagaatctggaaaagaccgaggcgtactttttaggaatattttgtgtagaagcctcgttaaaaatcttagccttaggttttgttttacacaggggatcgtatcttaggaacgtttggaacatcatggattttttcgttgtagtaactggtatcatcacgcagctgccgatcgcgccagccgacgtcgacttcaggaccttgcgtgccattagggtgctgaggccccttaaattagtatcgggcgttcctagtctgcaagtggtactgaagtccatcataaaggcgatggcgccgttgctgcagatcggcctcctggtgcttttcgcgatagtcatcttcgctatcatcggcctcgagttctactcaggggcgctgcataagacttgttataatttagaagatattagtgaaatagtaaatgaaggcgatagtgcgacgccgtgtaacgcggacaacgtgagtttagcaccatttggggcaaacgtgtgtgattatgagaagagcacgtgtttagagaaatgggaggggccgaacaggggtattacgtccttcgacaacatcggcttcgctatgctcaccgtcttccagtgcattaccatggagggctggaccgcaatcctctattggacgaatgacgcgctaggtagtgcgttcaactggatttactttgtgcctctcatagtattgggttcattctttatgctcaacttagttctcggtgtccttagcggtgagttcgctaaagaaagagagaaagtagaaaatagacaagaatttcttaaattaagaagacagcagcaactcgagagagaactcaatggttacgttgagtggatttgtaaagcagaggaagtaatattagcagaagaaagaacaacagaagaagaaaaaatgcacataatagaagcacggagaagagcagcggccaaaaagaagttaaaaaaccttggtaaaagtaaaagcacagatacagaagaggaagaacaagatgaagactgcggtgatgacggttttctaaaaagcaaagctcggtcagccgggaggtttgcggacttctggcgggctgagaagaggtttcggttttggatcaggcacacagtgaagacccagtggttctactggttcgtcattgtcttggtgctcttcaacacgatatgcgtcgctgtagagcattatagacaacccaagtggctgacttcgtttttatactatgccgaatttgtgttcctggggttgttcatgatggagatgtgggtgaagatgtatgcgcttgggccgcgaatctacttcgagtcgtccttcaaccggttcgactgcgtggtcatctccggctccatcttcgaggtcgtgtggtctgaggtcaagggtggctccttcggtctatctgtcctgagagctctaagactgttgaggatatttaaggtcaccaagtactggtcatcgctccggaacctggtgatatctctcctcaactcaatgagatccatcatctcgctgctgttcctgctcttcctgttcattctcatcttcgcactgctcggcatgcagctgttcgggggacagttcaacttcgaggacggcacgccgccgaccaacttcaacacctttcctatcgcgttgttaactgtcttccagatcctaacaggtgaagattggaacgaagtgatgtatgacggcatccagtcacagggcggcatccagagaggcatgatctactctctatactttgtcatcctcgtcttatttggcaactacacgctgctgaacgtgttccttgctatcgctgtcgacaacttggctaacgcccaggaattgacggcggcagaagaggaacaagtcgaggaggacaaggagaaacagctccaggaattggagaaagggatgggtgcattacacgcggtggacggcactccaccgggagtagatctaagtccctcttcgccgacgagtaggaagaacaaaaagaaagaagaggccaaaaaagaagatgaagatgaggtaccagatggaccaaaaccaatgctgccatattcgtccatgtttattttgtcacctactaatccaattaggcgaggcgcacactgggttgtaaatttaagatattttgatttttttatcatggtagttatatgtatgagttctgcggctttagcggctgaagaccccgtagtggaagagagtgacaggaacaaaatcctgaactacttcgattacgcgttcacgggcgtgttcaccgtggagatgctgctgaagatagtggacctcggcatcctgttccacccgggcgcctacctgcgcgacctgtggaacatcatggatgccgccgtcgtcatatgcgcccttgtcagcttcggatttgagatcggaggcgtgaaaaagggggcggggcagaatctgtccacaataaaatcgttaagagtgttacgagtgctcagacctttgaaaactataaaacgagttccaaagttaaaagcagtgtttgactgtgttgtgaactctttgaaaaacgtcattaacattctcattgtgtacatattgtttcaattcatattcgctgtaattgcagttcaactttttaatggtaaattttttcactgcaacgatatcagtaagaatacttttgaagactgccaagggtcgtatttcgtgtacgagtcaaacagcttgctgccgaaagtcaaccagcgcacgtggacgacgcaatccttccattacgacaacgtcgccgtggctatgcttacgctgttcgccgtgcagactggggaggggtggccacaagtattacaaaattcaatggccgccacctacgaagacaggggacccatacaaaattttcgaatagaaatgtccatattttatatagtttacttcgtggtgtttcctttcttctttgttaacatattcgtagctctgataattatcacatttcaagagcagggcgaagctgagcttcaggatggtgaaattgataagaatcagaaatcgtgtatagacttcacgatagaagcgcgacctctcgagaggtatatgccaagcaaaagggcgagttttaagtacaaagtgtggagaatagttgtctctacgcccttcgagtacttcatcatgacgctgatcgtcctcaacacattgttgctcatgatgaagtttcacgaggctccaccactactcatggacatattaacattcatgaacctcgtctttacgaccttcttccttctcgagaccgtattgaagctgatcgccttcgggtgtacgaattttttcaaagacccttggaatacattcgattttattacggtcattggaagtattattgacgccctcattatggagtttggcgagaacacattcaacgtcggtttccttcgcctgttccgagccgcgcgactgatcaagctgctccgacagggctacactattcggatactgctctggacattcgtgcagagtttcaaagccttaccctacgtgtgccttctcatcgcgatgctattcttcatctacgccatcatcgggatgcaggtgtttggcaatatagaattaacaccagagtctgacatgaacagacacaacaattttcgaagcttcattcaagcactcatgctactgttcagatgcgcaacgggcgagtcgtggcccaacataatgttggcttgccgcaaacccgccaagtgcgacatagcagctggaaaggcctccaacgaagaatgtggaagtacgctcgcctacgcctacttcgtatcttttatattcttctgttcgtttcttatgttgaatttgttcgttgctgttattatggataactttgactacctaacgagggactcgtccatcctcggcgcacatcatcttgatgaatttgttagaatatgggcagaatatgatccaaacgccacgggtaagatccattatacagaaatgtatgatatgttgaagaatatggatccgcctctggggtttggtaacaaatgtccaaatagactagcatataagaagcttattagaatgaatatgccgctagacgatgaggggaaagttaattttacaacaacactatttgccttaatacgagaaaacttgaacatcaaaatgagatctcccgaggaaatggaccaagcagatgaggaattaagggaaacaataacccacatttggccattacaagcgaagaagatgctcgacctgctggtgcctcgaaacgatgtactcaacgctggaaaactgaccgtcgggaagatatacgctggacttctaatcctcgagagttggagatctacaaggttcaagcagaatggtgttccggtactggaactacaaggatcacaccacgcctcaatggagtcgttggacgagggacggttacaagctcctcacacgtaccagaatggacaccaccatgggagatcatccagtttaagacgaacgcccagtccaagaagacgaggccactacggaggttatcatcacgatatcggattctcagacaccgtcagcaacgtcgtcgagatagtaaagcatgaacaccagagacacgggcgaacgcacagagcgccccactactaccacccacatgtttgggccccgataggtgagcgggagcgggaccgcgagtggcgggagtggcgcgaccgctcctgggagcgcgagggcgcgcgccgcggccgcaggcgccggttgccgcccacgcccaacaaaccgtcgtcgctgcagctccggccgccgcaccactcggcctcgataaactttccgaagctgaacgcgtcgccgacgcgcggccaaccggcctcgctgcccaaaccgccgcccgtcaacatgtcgtccacgtcgacggccgcggccgtggcccggcagagcatggccatgaccgccatgccgccgctcagcttcgagcaggccatgtcgatcggccgcagtggccggctactgccgtcgccggtgcgcaacgggtacgcgtcgtcgtcgtccaagcagcagcagcagcagcagcagccgtcgtcgtcgtcgtcggtgtccgcgatcaggcggcaaaagagcggcggcggtggccgggtgagcagccggcacagcgattcggacgaggacgactggtgctaa

SEQ ID NO: 6 - protein, 1764 aa, Heliothis virescens and Aphis gossypii
MGGAHQPATPGPSSLFIFADENPIRRYTKFIIEWPPFEYAVLLTIIANCVVLALEEHLPNGDKTILAQNLEKTEAYFLGIFCVEASLKILALGFVLHRGSYLRNVWNIMDFFVVVTGIITQLPIAPADVDFRTLRAIRVLRPLKLVSGVPSLQVVLKSIIKAMAPLLQIGLLVLFAIVIFAIIGLEFYSGALHKTCYNLEDISEIVNEGDSATPCNADNVSLAPFGANVCDYEKSTCLEKWEGPNRGITSFDNIGFAMLTVFQCITMEGWTAILYWTNDALGSAFNWIYFVPLIVLGSFFMLNLVLGVLSGEFAKEREKVENRQEFLKLRRQQQLERELNGYVEWICKAEEVILAEERTTEEEKMHIIEARRRAAAKKKLKNLGKSKSTDTEEEEQDEDCGDDGFLKSKARSAGRFADFWRAEKRFRFWIRHTVKTQWFYWFVIVLVLFNTICVAVEHYRQPKWLTSFLYYAEFVFLGLFMMEMWVKMYALGPRIYFESSFNRFDCVVISGSIFEVVWSEVKGGSFGLSVLRALRLLRIFKVTKYWSSLRNLVISLLNSMRSIISLLFLLFLFILIFALLGMQLFGGQFNFEDGTPPTNFNTFPIALLTVFQILTGEDWNEVMYDGIQSQGGIQRGMIYSLYFVILVLFGNYTLLNVFLAIAVDNLANAQELTAAEEEQVEEDKEKQLQELEKGMGALHAVDGTPPGVDLSPSSPTSRKNKKKEEAKKEDEDEVPDGPKPMLPYSSMFILSPTNPIRRGAHWVVNLRYFDFFIMVVICMSSAALAAEDPVVEESDRNKILNYFDYAFTGVFTVEMLLKIVDLGILFHPGAYLRDLWNIMDAAVVICALVSFGFEIGGVKKGAGQNLSTIKSLRVLRVLRPLKTIKRVPKLKAVFDCVVNSLKNVINILIVYILFQFIFAVIAVQLFNGKFFHCNDISKNTFEDCQGSYFVYESNSLLPKVNQRTWTTQSFHYDNVAVAMLTLFAVQTGEGWPQVLQNSMAATYEDRGPIQNFRIEMSIFYIVYFVVFPFFFVNIFVALIIITFQEQGEAELQDGEIDKNQKSCIDFTIEARPLERYMPSKRASFKYKVWRIVVSTPFEYFIMTLIVLNTLLLMMKFHEAPPLLMDILTFMNLVFTTFFLLETVLKLIAFGCTNFFKDPWNTFDFITVIGSIIDALIMEFGENTFNVGFLRLFRAARLIKLLRQGYTIRILLWTFVQSFKALPYVCLLIAMLFFIYAIIGMQVFGNIELTPESDMNRHNNFRSFIQALMLLFRCATGESWPNIMLACRKPAKCDIAAGKASNEECGSTLAYAYFVSFIFFCSFLMLNLFVAVIMDNFDYLTRDSSILGAHHLDEFVRIWAEYDPNATGKIHYTEMYDMLKNMDPPLGFGNKCPNRLAYKKLIRMNMPLDDEGKVNFTTTLFALIRENLNIKMRSPEEMDQADEELRETITHIWPLQAKKMLDLLVPRNDVLNAGKLTVGKIYAGLLILESWRSTRFKQNGVPVLELQGSHHASMESLDEGRLQAPHTYQNGHHHGRSSSLRRTPSPRRRGHYGGYHHDIGFSDTVSNVVEIVKHEHQRHGRTHRAPHYYHPHVWAPIGERERDREWREWRDRSWEREGARRGRRRRLPPTPNKPSSLQLRPPHHSASINFPKLNASPTRGQPASLPKPPPVNMSSTSTAAAVARQSMAMTAMPPLSFEQAMSIGRSGRLLPSPVRNGYASSSSKQQQQQQQPSSSSSVSAIRRQKSGGGGRVSSRHSDSDEDDWC

SEQ ID NO: 7 - DNA, 2068 bp, Heliothis virescens
tgttaagtgtatacagtgaaatgtgcgacttatgacgaaggctcaaccactatggatcactttaggcggcgaccccgctacaagctccgaaggggctcagcagattcaaactattcgcaaccatcgtcggagctgtcgcttgacgaagacaaggaggcgctgcgccgcgagaaggaagcgcaggctatcaaacagctggacaaggcacgggagaagccagtggcgttcgcggtgcgcaccaatgtgtcatacgatggcaccgtggacgacgattcgccggtgcacggcagcgcgatctccttcgaggtgcgcgattttctccacatcaaggagaagtacgacaacaactggtggatcgggcggctcgtgaaggagggcagcgacgtgggcttcatcccgtcgcccgtgaagctggaggccgtgcggtcggggctgggcgcgcggcgctacccgcggcccgcgtccgccgcgccgcacgcgccgcacgcgccgcccctctccagaggtagcaccccccccacgcccggcgaggagtcggacggcgcggggcgcgggcggggcgggggcgcgggggggaaggagaagcgcaagccgttcttcaagaaggcggaggcgtcgacgccgtacgacgtggtgccctccatgcggcccgtcgtcatcgtggggcccagcctcaagggatacgaggtcaccgacatgatgcagaaggctttgtttgattttctcaaaagacgctttgaaggcaggataataataacgcgcgtgatggccgacatatctctcgcgaagcgctctctgctcaacaacccgtcgaaacgagccatcatggagcgttccaactcgcgctccacttgcctcgctgaagtgcaggctgaaatcgagaggatattcgagctggctcggactctccaattggtagtgctggactgtgacaccatcaaccacccctcacagctcgccaagacctcgctggctcccaccatcgtgtacctgaaaatctccagtcccaaggtgctacagcggctgatcaagtcgcgggggaaggggcagaccaagaacctgtcggtgcagatggtggctgctgagaagctggcgcagtgcccgcctgacatgttcgacgtgatccttgatgagaaccagctcgaggatgcttgcgaacatatagctgaatatttagaggcgtactggcgcgcgacgcacccgccggtgccggaggaggcggcgcccgcgcgccccgtgccgcgcacgcacaacgcgcacgccatgcctggcggcaggtcccgcgcggcgcggccggagcgcctggaggaggagtactacccgcgcggcgagcgctgcgagcggtacgagcgcggctacccgcgcgactaccacgagtacgggcccgagtacccgcgggagcgcgcgcacgcggcgcacgcggcggcgccgcacgacgcgtacgggcacgactacacgcgcgacgcgtacgcgcgcgaggagtacgggcgcgagtacgcgcgcgaggagcgcgagcggcggcgcgagcgcgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtagtgccgcccgcctcgcgcacgccgtgccgcaccgcgccgcgccgtgccgcatgccgtgccgcgtcccgctgcgccgccgctgcttgcggcaattgtataaattttacatacagaacgtaagaggggctaagtttagtaccgttagatattatccgtttcccattttttgaacatactctgtaattatattgcatcgtctgtacttcattgttgcaatcatttgtaaataaaatactttatatcgaaaaaaaaaaaaaaaaaaaaaaaaaaaaaaaaaattaaaaaaaaaaaa

SEQ ID NO: 8 - protein, 509 aa, Heliothis virescens
MDHFRRRPRYKLRRGSADSNYSQPSSELSLDEDKEALRREKEAQAIKQLDKAREKPVAFAVRTNVSYDGTVDDDSPVHGSAISFEVRDFLHIKEKYDNNWWIGRLVKEGSDVGFIPSPVKLEAVRSGLGARRYPRPASAAPHAPHAPPLSRGSTPPTPGEESDGAGRGRGGGAGGKEKRKPFFKKAEASTPYDVVPSMRPVVIVGPSLKGYEVTDMMQKALFDFLKRRFEGRIIITRVMADISLAKRSLLNNPSKRAIMERSNSRSTCLAEVQAEIERIFELARTLQLVVLDCDTINHPSQLAKTSLAPTIVYLKISSPKVLQRLIKSRGKGQTKNLSVQMVAAEKLAQCPPDMFDVILDENQLEDACEHIAEYLEAYWRATHPPVPEEAAPARPVPRTHNAHAMPGGRSRAARPERLEEEYYPRGERCERYERGYPRDYHEYGPEYPRERAHAAHAAAPHDAYGHDYTRDAYAREEYGREYAREERERRREREPPDDYGPSRRALNAV

SEQ ID NO: 9 - DNA, 2116 bp, Heliothis virescens
gctcgcgaccgtcgcatcggacggtagtgcggcagctccgatgtgccgccaccacggccgaccacgacgcgatcccactggtggtccgcagtggccaccacctgccgccatgtcgtaacatgatggggtacaacaaccaagggcacgggtacaacaagttgtttacacagggctcagcagattcaaactattcgcaaccatcgtcggagctgtcgcttgacgaagacaaggaggcgctgcgccgcgagaaggaagcgcaggctatcaaacagctggacaaggcacgggagaagccagtggcgttcgcggtgcgcaccaatgtgtcatacgatggcaccgtggacgacgattcgccggtgcacggcagcgcgatctccttcgaggtgcgcgattttctccacatcaaggagaagtacgacaacaactggtggatcgggcggctcgtgaaggagggcagcgacgtgggcttcatcccgtcgcccgtgaagctggaggccgtgcggtcggggctgggcgcgcggcgctacccgcggcccgcgtccgccgcgccgcacgcgccgcacgcgccgcccctctccagaggtagcaccccccccacgcccggcgaggagtcggacggcgcggggcgcgggcggggcgggggcgcgggggggaaggagaagcgcaagccgttcttcaagaaggcggaggcgtcgacgccgtacgacgtggtgccctccatgcggcccgtcgtcatcgtggggcccagcctcaagggatacgaggtcaccgacatgatgcagaaggctttgtttgattttctcaaaagacgctttgaaggcaggatcatcatcacgcgcgtgcagtctgatatatcattggccaaacggacgcaaaacgcgcccaagaaaccgttcgtagagagggccggtaacaggaccaactgtttagccgaggtccaggctgaaatcgagaggatattcgagctggctcggactctccaattggtagtgctggactgtgacaccatcaaccacccctcacagctcgccaagacctcgctggctcccaccatcgtgtacctgaaaatctccagtcccaaggtgctacagcggctgatcaagtcgcgggggaaggggcagaccaagaacctgtcggtgcagatggtggctgctgagaagctggcgcagtgcccgcctgacatgttcgacgtgatccttgatgagaaccagctcgaggatgcttgcgaacatatagctgaatatttagaggcgtactggcgcgcgacgcacccgccggtgccggaggaggcggcgcccgcgcgccccgtgccgcgcacgcacaacgcgcacgccatgcctggcggcaggtcccgcgcggcgcggccggagcgcctggaggaggagtactacccgcgcggcgagcgctgcgagcggtacgagcgcggctacccgcgcgactaccacgagtacgggcccgagtacccgcgggagcgcgcgcacgcggcgcacgcggcggcgccgcacgacgcgtacgggcacgactacacgcgcgacgcgtacgcgcgcgaggagtacgggcgcgagtacgcgcgcgaggagcgcgagcggcggcgcgagcgcgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtagtgccgcccgcctcgcgcacgccgtgccgcaccgcgccgcgccgtgccgcatgccgtgccgcgtcccgctgcgccgccgctgcttgcggcaattgtataaattttacatacagaacgtaagaggggctaagtttagtaccgttagatattatccgtttcccattttttgaacatactctgtaattatattgcatcgtctgtacttcattgttgcaatcatttgtaaataaaatactttatatcgaaaaaaaaaaaaaaaaaaaaaaaaa

SEQ ID NO: 10 - protein, 510 aa, Heliothis virescens
MMGYNNQGHGYNKLFTQGSADSNYSQPSSELSLDEDKEALRREKEAQAIKQLDKAREKPVAFAVRTNVSYDGTVDDDSPVHGSAISFEVRDFLHIKEKYDNNWWIGRLVKEGSDVGFIPSPVKLEAVRSGLGARRYPRPASAAPHAPHAPPLSRGSTPPTPGEESDGAGRGRGGGAGGKEKRKPFFKKAEASTPYDVVPSMRPVVIVGPSLKGYEVTDMMQKALFDFLKRRFEGRIIITRVQSDISLAKRTQNAPKKPFVERAGNRTNCLAEVQAEIERIFELARTLQLVVLDCDTINHPSQLAKTSLAPTIVYLKISSPKVLQRLIKSRGKGQTKNLSVQMVAAEKLAQCPPDMFDVILDENQLEDACEHIAEYLEAYWRATHPPVPEEAAPARPVPRTHNAHAMPGGRSRAARPERLEEEYYPRGERCERYERGYPRDYHEYGPEYPRERAHAAHAAAPHDAYGHDYTRDAYAREEYGREYAREERERRREREPPDDYGPSRRALNAV

SEQ ID NO: 11 - DNA, 2282 bp, Heliothis virescens
ggctctgcagccttcaggcctccctgaaaactgtgaaaattcattagaaaagtgcatatttatcatacttgtgttgtactaaactgtgcacccgttgtacatagtggatattgtttgtgaaaactgggcgtaacattgtatgctgtagcggcggcggtgccgtggggggcatgcgccggcggcgctgaggcatgagcgcggcgcgcgcgccggatccccgcgacccaccgcgccaccaccgccaacaccctcagggctcagcagattcaaactattcgcaaccatcgtcggagctgtcgcttgacgaagacaaggaggcgctgcgccgcgagaaggaagcgcaggctatcaaacagctggacaaggcacgggagaagccagtggcgttcgcggtgcgcaccaatgtgtcatacgatggcaccgtggacgacgattcgccggtgcacggcagcgcgatctccttcgaggtgcgcgattttctccacatcaaggagaagtacgacaacaactggtggatcgggcggctcgtgaaggagggcagcgacgtgggcttcatcccgtcgcccgtgaagctggaggccgtgcggtcggggctgggcgcgcggcgctacccgcggcccgcgtccgccgcgccgcacgcgccgcacgcgccgcccctctccagaggtagcaccccccccacgcccgatgtgccatcgttcgatcgaggcgaggagtcggacggcgcggggcgcgggcggggcgggggcgcgggggggaaggagaagcgcaagccgttcttcaagaaggcggaggcgtcgacgccgtacgacgtggtgccctccatgcggcccgtcgtcatcgtggggcccagcctcaagggatacgaggtcaccgacatgatgcagaaggctttgtttgattttctcaaaagacgctttgaaggcaggatcatcatcacgcgcgtgcagtctgatatatcattggccaaacggacgcaaaacgcgcccaagaaaccgttcgtagagagggccggtaacaggaccaactgtttagccgaggctgaaatcgagaggatattcgagctggctcggactctccaattggtagtgctggactgtgacaccatcaaccacccctcacagctcgccaagacctcgctggctcccaccatcgtgtacctgaaaatctccagtcccaaggtgctacagcggctgatcaagtcgcgggggaaggggcagaccaagaacctgtcggtgcagatggtggctgctgagaagctggcgcagtgcccgcctgacatgttcgacgtgatccttgatgagaaccagctcgaggatgcttgcgaacatatagctgaatatttagaggcgtactggcgcgcgacgcacccgccggtgccggaggaggcggcgcccgcgcgccccgtgccgcgcacgcacaacgcgcacgccatgcctgacccccctgctaacactggaggacatcgcgagagatccgactcctatagctacgagagagaaagaggcggcaggtcccgcgcggcgcggccggagcgcctggaggaggagtactacccgcgcggcgagcgctgcgagcggtacgagcgcggctacccgcgcgactaccacgagtacgggcccgagtacccgcgggagcgcgcgcacgcggcgcacgcggcggcgccgcacgacgcgtacgggcacgactacacgcgcgacgcgtacgcgcgcgaggagtacgggcgcgagtacgcgcgcgaggagcgcgagcggcggcgcgagcgcgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtgagccgccggacgactacgggccgtcgcgccgcgcgctcaacgctgtgtagtgccgcccgcctcgcgcacgccgtgccgcaccgcgccgcgccgtgccgcatgccgtgccgcgtcccgctgcgccgccgctgcttgcggcaattgtataaattttacatacagaacgtaagaggggctaagtttagtaccgttagatattatccgtttcccattttttgaacatactctgtaattatattgcatcgtctgtacttcattgttgcaatcatttgtaaataaaatactttatatcgaaaaaaaaaaaaaaaaaaaaaaaaaa

SEQ ID NO: 12 - protein, 541 aa, Heliothis virescens
MSAARAPDPRDPPRHHRQHPQGSADSNYSQPSSELSLDEDKEALRREKEAQAIKQLDKAREKPVAFAVRTNVSYDGTVDDDSPVHGSAISFEVRDFLHIKEKYDNNWWIGRLVKEGSDVGFIPSPVKLEAVRSGLGARRYPRPASAAPHAPHAPPLSRGSTPPTPDVPSFDRGEESDGAGRGRGGGAGGKEKRKPFFKKAEASTPYDVVPSMRPVVIVGPSLKGYEVTDMMQKALFDFLKRRFEGRIIITRVQSDISLAKRTQNAPKKPFVERAGNRTNCLAEAEIERIFELARTLQLVVLDCDTINHPSQLAKTSLAPTIVYLKISSPKVLQRLIKSRGKGQTKNLSVQMVAAEKLAQCPPDMFDVILDENQLEDACEHIAEYLEAYWRATHPPVPEEAAPARPVPRTHNAHAMPDPPANTGGHRERSDSYSYERERGGRSRAARPERLEEEYYPRGERCERYERGYPRDYHEYGPEYPRERAHAAHAAAPHDAYGHDYTRDAYAREEYGREYAREERERRREREPPDDYGPSRRALNAV

SEQ ID NO: 13 - DNA, 4383 bp, Aphis Gossypii
gtatcgatcggtccgcggctggccggccgacagtgcgatgcacagtgtcattagtgttccagcgacggcgacgggagacggacatcggcggtctaatcaaaacaaacccgataacataataatataatatcatattattttcatatatcataatataataataattattgtcgctggtattatatagtattaattacgttcgtgctcgtccggttcaacaatatcataatcgtcactgtaaaatattataatatatatttactttaaatcttcgttcctctcgacgacttactatacactataatttaatatttttatcactatttattaacgttatatacccataaaaatataatacaaaagtaaattatatcatattatatactataattttattgtaatataacgttgtacaaattgccgcgtatttagtaactatatatattacgagtatattatatatagtatattatccttattgtaaagacagacaataacatgaaatccgccaaagtcttggtcgccctattcctatacgggtgtttcatggttcgggaatgcattcaacacgacaataacgaacccgtcaaaacggctctgctgcaaacgtgggctgaaaaattatattttgaattgtggaactgcggagaatatataacaaatcgaaaatcactgcaaaccaaatataacaaggaatcaaaattagaagcaaggaatgggcaaattttggtagcaaatatttcgagtgttatcaaatcaatgatggataaaaaacaacaatctttaaaaagaattttagaaagcgttgaacaagttgcagtttcagcacacgacgatgaacctccgggcaatgattataagattcggaattcaaaaccaaaaaaagctaccccagataccaacatattattaaatgatctaaaagaaaattctcatttttacaatacgcctgtaaatactacttatagttctgtacacataccaacttatgtttatgaccgggccactgaagtaattaaggctatcaaatggtcagaaaacctggatgcgacatttaaaaaaaactacgaaaccgatccaagattatcgtggcaatattttggtagtacaactggtttcatgcgacaatttccagcaatgcaatggccagaggaaccagaagatttgtatgactgtcgaatgcgaccttggtacgttgaagcagcagccagtcctaaggatattcttatattagtcgataattctgggtcaatgatgggacaaagtaaaatcattgccagacatgtgataaatacattattggatacattatcagtgaacgactacgtgaatgttcttgtatttagtaatgtaacgaatgaagtagtgccatgtttcaaaggtttactagtacaagccacacttgcaaatatccgagaactaaaattaggcgtagaaaatattgccgatccaaataatatctcagattttagcgaagcattaaccagggcgttcgacacactggaaacgtaccgcgaacgaaatatgggtgctatgtgtaatcaggccatcatgttggttactgatggagtaccagaaaacttctacgaattatttaaatcacataattggaagaatggtacaatgggtatgccagtaagagtatttacgtatctaattagtcgagaactgcctgaaataagagacatgcaatggatggcttgtgcgaatcatgggtattttgttcatttaagtacggtagaagaagttcgagaacaagtggtacactatttaccggtaatggctagaccattggtgttacaccaatctcatcatccggttatttggacgccgttatacgctgacgttactgatccaaaaatgactgattatttgtgggaaaaagaagaatgtcatcaacagtataatgatacgataagctataaaaaaacaaaagacaaatttttctatccaaagaatatagcaaaacgagaacaagatcgaaaaagaaaagaattggatcaaaatgtaaacgtaaatagacaaaaaaaacacaatttaataacgtctcttgcgatgccagcattcgacaagaaaactgaaatgacaaaagtggcccatttacttggtgttgttggaactgatgttcccgaagctgatctacgagaagcaatgtcacctcacatattgggtgtcaataactatgcatttatcgttactaataacggttttattgtcacacatccagatctcagaccagtttttggagatattttgaagccaaattacaatagcatcgatgtgactgaagtggaacttgtcgaatccgacaataacgcaagggtatttgatcgaagtatactcacgcttcgtgattatatcatcaatcaaacaactggcgatcgagaaatcactgtgaaatatcattacgacaatataagacgtgcaacaacagcagagagacattattattattcgattgttgaaggaactccatttacagttgtggttgctttacaagaaaagcacttcggttacagagttaaaataccagaaagatttcaaactcttaatacaacaagaacaacccttttggaccttttcaaagatgatgagtggagaattcatccagattggctgtattgcagatatgcatacgatgatggtgataatacgtcatttaaaactccagaagatgaacttaaacattttttaaagagaatcagtaaaaccgataattcttggaataaatggcctcctccgagattttatagtgaatcatacgactcaaacgggtcaatattgcccgaagaaaaattcgattgcaaatgttattgtgacaaagaacttatgcttagtttgatatacgatgccaacatgactaaaggtatttttacagaagctaaaaatgatacagataagaaaaaaaatccagcagcggtcttgatgagtttattaccgagaaccgaatttgaaaagagatttggagtgacattagcttttgttgctacccatagtggtctcaccagatggatggattatgaagataaacccaagacgtcatacaattctgataaccatgaaaatcaacaaccgcatttcattgacgaaaatgtttactcaatcgaagaagtttggtatagaagggctgtcgagtataatatggtaaacgatgaaagctatgtatattctgtaccatttgaaaatgaaaatattaaatcaacattggtaacggcgagtcaagcggttttcgttactagtaaagaaaataaaaagaaaggtgctgctgcagttgtcggctatcaattcagacactcggctcttcaagaattgttttcgaacattacaaaacagtgttactcagaaactggttgtacgccggcaaaggacacttgtcaatccttagaacgggattgctacatacttgacaataatggttatattatagcatcagaaaatccaatggatactgggaagttcttcggtaaagtaaaaggaccaatcatgaatatgatggtagaagaaaaaatttacaaaaaaataacaatatttgattatcaaggagtatgttttaagcgagaaaatgaagatgattcaaatagttcgtcatttttgtatactccgatacatcatatgaaacaattaattggatggctaatagggcgtgtattatggttggccgctcattcaaattttcagtggatggtgtctcaggctcaagccatattcgaagacgattaccaagaagatgaagaaagtaattacagttcagatactatttccacttttaagattgaaggcgaaaaagcattaaaatacgttaaaattaatcgaactcgacctaagtcttgcgatcgtgtcgtcgatttgtttacgctaacttcgaaaattccagaaaaggttgaacgtaaatctatgtgtgccaggccttatgaattgcgaaaaataatgaacaccaaccttatcttgctggtcgtcaacggacggtgtccagaatcgtctaaatccgaaaattttgacaccttaccagaagaagttatgtatgacgagttcaataattcgttgggttgtcacaaagcgttttatagtttaccacgacgtagacctaaaacagcttgtattaggacacatccagacgagcacgaaatttatgacaaaatgtgtggcagcagtacaataattacaattacaccaataactttgtcagtaatttttttttctacgtttaatttgataaggttttttgtaattttttaattctaaatcaaaaatttaaattctcttacttatgtattgataaatattgttcaagaagttatcttcgatgttaaatattttgtctattgcgtttattacaatataatatacgttaaatcttgaaaaaaaaaaaaaaaaaaaaaaaaa

SEQ ID NO: 14 - protein, 1245 aa, Aphis gossypii
MKSAKVLVALFLYGCFMVRECIQHDNNEPVKTALLQTWAEKLYFELWNCGEYITNRKSLQTKYNKESKLEARNGQILVANISSVIKSMMDKKQQSLKRILESVEQVAVSAHDDEPPGNDYKIRNSKPKKATPDTNILLNDLKENSHFYNTPVNTTYSSVHIPTYVYDRATEVIKAIKWSENLDATFKKNYETDPRLSWQYFGSTTGFMRQFPAMQWPEEPEDLYDCRMRPWYVEAAASPKDILILVDNSGSMMGQSKIIARHVINTLLDTLSVNDYVNVLVFSNVTNEVVPCFKGLLVQATLANIRELKLGVENIADPNNISDFSEALTRAFDTLETYRERNMGAMCNQAIMLVTDGVPENFYELFKSHNWKNGTMGMPVRVFTYLISRELPEIRDMQWMACANHGYFVHLSTVEEVREQVVHYLPVMARPLVLHQSHHPVIWTPLYADVTDPKMTDYLWEKEECHQQYNDTISYKKTKDKFFYPKNIAKREQDRKRKELDQNVNVNRQKKHNLITSLAMPAFDKKTEMTKVAHLLGVVGTDVPEADLREAMSPHILGVNNYAFIVTNNGFIVTHPDLRPVFGDILKPNYNSIDVTEVELVESDNNARVFDRSILTLRDYIINQTTGDREITVKYHYDNIRRATTAERHYYYSIVEGTPFTVVVALQEKHFGYRVKIPERFQTLNTTRTTLLDLFKDDEWRIHPDWLYCRYAYDDGDNTSFKTPEDELKHFLKRISKTDNSWNKWPPPRFYSESYDSNGSILPEEKFDCKCYCDKELMLSLIYDANMTKGIFTEAKNDTDKKKNPAAVLMSLLPRTEFEKRFGVTLAFVATHSGLTRWMDYEDKPKTSYNSDNHENQQPHFIDENVYSIEEVWYRRAVEYNMVNDESYVYSVPFENENIKSTLVTASQAVFVTSKENKKKGAAAVVGYQFRHSALQELFSNITKQCYSETGCTPAKDTCQSLERDCYILDNNGYIIASENPMDTGKFFGKVKGPIMNMMVEEKIYKKITIFDYQGVCFKRENEDDSNSSSFLYTPIHHMKQLIGWLIGRVLWLAAHSNFQWMVSQAQAIFEDDYQEDEESNYSSDTISTFKIEGEKALKYVKINRTRPKSCDRVVDLFTLTSKIPEKVERKSMCARPYELRKIMNTNLILLVVNGRCPESSKSENFDTLPEEVMYDEFNNSLGCHKAFYSLPRRRPKTACIRTHPDEHEIYDKMCGSSTIITITPITLSVIFFSTFNLIRFFVIF

SEQ ID NO: 15 - DNA, 5028 bp, Heliothis virescens
tgcccgccccgcgctctctcgcgcctgcgcactacaccgcatacctatcaaccttatcaatgttcgtcgcccagtccctctcgacggtctcacacatcggaatccctaaacatcatcaatgtcataattagattagaaacgtaacatgttctgccgcaattctagtcagtgaactcaataacgcagtggttctgatgtgcaagtgtgtcggcgccttcgtgttacttctgctggttttgttcctggactccagggactgctcggctagtgcacaatatgatgccgctggcaaaatatcgctcaactcggtccaagcatgggcggttaagcttggtactgagctgtatcacttcggagaatttatcaccaggaagaaagaggtgcaggatagttttaaatccgctcaagtggaatcgagagatggggaaaaactagttcagagtatgtcagacgacatacgcgccatgatggaacttaagatcagcgccgtcaaaaggatagtggaagctgctgaaaacatggcgttcgataaacaaaatgagcccgtacctgaagattttcagttctacaatagcaaagaaatggaggaaccttgggacgaggtgtcaatcacaacaacgcctgagacagacttcggtcaggaaagttggatagtacgaccgccgtcaaagaatgcgcacacgcaacaaaacccacacttctcaaacattccagtaaacactaattttagcagtgtacatgttccaacaaatgtttatgcttgggcacctgaagttatcaagggaatccactggtcagagggactggacacgcacttcatcaataattaccagagcgacccgacgttatcgtggcaatacttcggcagctctactggattcatgagacattatcccgcattgaaatggagggctgaccctgtggatatttatgattgtcgaacgcgagcgtggtacatggaagctgcagcgagtccaaaggatgtaatcattttagttgatcgtagcgggtcaatgaccgggcaaaggcgggacatcgccaaacacgtcgttaccaatatactggatacgctgggcaacaacgattttgtcaacgttctgacttttgctgacaccgtagaagaaatcgtaccgtgttttgaagattctctagtgcaggccactctgggaaatatacgggaactaaagttggcgatggacagttttgaaacgcaggagatagcaaacttttccgctgcgctgacgagagctttcgaactactggagatatacagaaataacagtggaggtgcaaattgcaaccaggctataatgctggtaacagacggcgtaccctacaattacaaagaaatatttgagaaatacaactggaagtatgagacccctgtgagagtgtttacgtacctgatagggcgtgaggtaaaggtcgcagacgtaagagaggtgaagtggatggcgtgcgcgaatcgcgggttctacgtgcatctgagtactctagcggaggtgcgcgagcgcgtgttggaacatgtgaacgtgctcgcgcgcccgctcgtgctgcagagagaaaagcatcctgtcgtctggacacccgtctacgctaacgtcactgatccgaaagtagcagattatctgtgggagcaacgtgagcgagcagagcagaaagaacgcttcatgagccagcggagggacaagaatctcttcaactcggacaaagaacaggatagacgatggagaattactcagatgaaacagggccagtacagtgagctcggcaattcccagtatcagttaatgacatcggtgtccatgccggtgtacgacctccgacacaacgagtccgtagaggagaacggccagaaagagatgcgtattgctagactgttgggaatcgctggcactgacgtacctctttctgaaatacaagctcttatgacaccgtataagatcggtgtgaatggctacgcgtttatagtaacgaacaatggttacatcttgatacacccggatctgaggcctgtgtttcaacaaatcttgaaaccaagctacaacagcgtcgatatgatagaagttgaactctttgacgatgacagaggccccaggaactttagcaaagagctcactgcactccgaaaagaaattatcgatcaaaagacgggcaacaaggtgatgagtgtcaagtaccatttggatgatatgaaaagagtatcccgagcaaaaaggcattatttctggacgggtataagcgactcccccttcacgttggttgtggcaataccagagaattacggccgccaccgcatcacgccaccaccgactgatgatatccacaggctgtctctcacatccaagaacatctcggcaaggcagtacctgtcggataagtggagcgttcatcctgactggatgtattgccggcattatgaacgcacgtttgcaactgctgaggatgagcttctctatttcttggagcgcgtcgctaagcctggctggcgctggcctgccaagcctcgtcccccggagcaccacaagaataaacctggacatgagcggcataacaacggaacaccagaaacgagagaaaggagcaaaatatccaatagcaccccaaggaatgagtattactgcgatcatggcttgatgcaggccttggtgtatgacgcgagaaatacagcttggtttaacaagagcatctctgattctgcatctgatgagaaagccccattgacaaaagtgatcggattgttgccgagggtggagttcattcaacggtttgggtacatcgtggcgttcctatcaacccacagcggactaacgcgctggcagacccatccacccaaggaacatgataatgacaaaccagatttcggcaagcaatggccacgagcgatcgaagaagtctggtaccggcgcgcagtcgagcagcactacgttgatcctctgagctatgtctacagtgtagacttgagcactgtcaagttccccctcaacgttagcctggctctggtcaccgcatcacatgccgtcttccacggagacgggcataggaaggctccagcggctgtcgttgggtttcagttcaaacatgagcggctcagtgaatggtttgataatattacgtcttcgtgtgaacataacaaggagtgtacaacttgccagtctgacaactgggattgttacctggtggacaacaacggctgggttattgttagtgaagattccacacagacaggccagttcttcggaaggattcgaccggacatcatgtcaagattagtggaagatgaggtgtacaaggcagtgcatattgttgattaccaggcagtctgcttcagagagaagaaaactacgaatccagcgacaatgctctttaccccacttgaaaacttgcgcctcgtgatggcatggttcttagctaccacggtctggttctacaactctataacgacaggtttggctcaagcatccagctactctttcgatgacgagaatgttacacccactgttgcataccagagctacgaaaacgatgaagagacggatgacccatacatgtctaaacctccaaacatgcgggtcttagaacgcgactatgagaagctggtgcttataaaccgaacgcggccaactccttgcgacagagagatgtacttgtaccaactagactataacaacttagacgagaagctgaacaagccagtggcggagtgcgggcgaccattcatagctcaacatgtcaactacacaaacatgctaatgattgtagtagacgccaattgcccgaaagacgaggtcgcagccatgtcggtggatgcgacagaagtgaactataacgagtcgctgccttgcctgaagcatatgcatcccttgtataggaagcagcctacttcatgcataaggaatcatactgaggagagcaacatcgacatgtgcggacgcggcagtctacccggcaacagcaagacatgggtttcgctgagcatctttttactagtcaaatattatctggcgtaaagtgtaggttcaaagctaagggaggtaaagcacaaagcggccagaatgggcaatgaaggtacaacaaagtcgatggcgagtgtaggaatgaatataaatcttctggctgcctgttctttatctctctggtattaccgttgaagaaatcctcactgaaatattagccattcttaggtaaattattaaataatgcatacggtaagttggagagccgggtattgttttaaatgaatattgaggcctggaactgcgagcgggcaaaggcgtggtttcaggcctaggtaatagattgaattatacgttttgttaaaatgtgtcatagtttatcggaatatttttcgtagtaaatccaaagatatatcgatggtaataacttttaattataatcgttgcttttgtttgattattattataattgtattgatctccgcttttagaataggtagatattataaaaaaatacatgtcattcccacatatcaaattttaaatgatacgagtatgctcccattgtgccacaataggaagcaatataaaaatatatcatatcaaaaattcaattacccccgtggtatgtgggggtcaatttttttaaaattgtgaacaaaataaaaaaaaacttttagtattaaattatatagcttttgtaaaacgtttggctgcatgattttaaattctcctttatttatgtaatgtttagatattttctttatttccaaatctaaatgttaaattatttccgtcctgctggtgaaaaggtttcacttattaatttggacaatatgcaaaattacaaacaattagggcaaatcttcaaattaatttgtttgcattgaaccagaggtattacaccactggttgaaataaatatttgtacctaatattactagaatttagtatttcgaatctctgtcatttaagtatttgtcttagaagtcaactatttactgaattactgaaatgtgattgtttgtttgtaataaataaatgattattaaaagtaaaaaaaaaaaaaaaa

SEQ ID NO: 16 - protein, 1271 aa, Heliothis virescens
MCKCVGAFVLLLLVLFLDSRDCSASAQYDAAGKISLNSVQAWAVKLGTELYHFGEFITRKKEVQDSFKSAQVESRDGEKLVQSMSDDIRAMMELKISAVKRIVEAAENMAFDKQNEPVPEDFQFYNSKEMEEPWDEVSITTTPETDFGQESWIVRPPSKNAHTQQNPHFSNIPVNTNFSSVHVPTNVYAWAPEVIKGIHWSEGLDTHFINNYQSDPTLSWQYFGSSTGFMRHYPALKWRADPVDIYDCRTRAWYMEAAASPKDVIILVDRSGSMTGQRRDIAKHVVTNILDTLGNNDFVNVLTFADTVEEIVPCFEDSLVQATLGNIRELKLAMDSFETQEIANFSAALTRAFELLEIYRNNSGGANCNQAIMLVTDGVPYNYKEIFEKYNWKYETPVRVFTYLIGREVKVADVREVKWMACANRGFYVHLSTLAEVRERVLEHVNVLARPLVLQREKHPVVWTPVYANVTDPKVADYLWEQRERAEQKERFMSQRRDKNLFNSDKEQDRRWRITQMKQGQYSELGNSQYQLMTSVSMPVYDLRHNESVEENGQKEMRIARLLGIAGTDVPLSEIQALMTPYKIGVNGYAFIVTNNGYILIHPDLRPVFQQILKPSYNSVDMIEVELFDDDRGPRNFSKELTALRKEIIDQKTGNKVMSVKYHLDDMKRVSRAKRHYFWTGISDSPFTLVVAIPENYGRHRITPPPTDDIHRLSLTSKNISARQYLSDKWSVHPDWMYCRHYERTFATAEDELLYFLERVAKPGWRWPAKPRPPEHHKNKPGHERHNNGTPETRERSKISNSTPRNEYYCDHGLMQALVYDARNTAWFNKSISDSASDEKAPLTKVIGLLPRVEFIQRFGYIVAFLSTHSGLTRWQTHPPKEHDNDKPDFGKQWPRAIEEVWYRRAVEQHYVDPLSYVYSVDLSTVKFPLNVSLALVTASHAVFHGDGHRKAPAAVVGFQFKHERLSEWFDNITSSCEHNKECTTCQSDNWDCYLVDNNGWVIVSEDSTQTGQFFGRIRPDIMSRLVEDEVYKAVHIVDYQAVCFREKKTTNPATMLFTPLENLRLVMAWFLATTVWFYNSITTGLAQASSYSFDDENVTPTVAYQSYENDEETDDPYMSKPPNMRVLERDYEKLVLINRTRPTPCDREMYLYQLDYNNLDEKLNKPVAECGRPFIAQHVNYTNMLMIVVDANCPKDEVAAMSVDATEVNYNESLPCLKHMHPLYRKQPTSCIRNHTEESNIDMCGRGSLPGNSKTWVSLSIFLLVKYYLA

SEQ ID NO: 17 - DNA, 4892 bp, Heliothis virescens
cggaatccctaaacatcatcaatgtcataattagattagaaacgtaacatgttctgccgcaattctagtcagtgaactcaataacgcagtggttctgatgtgcaagtgtgtcggcgccttcgtgttacttctgctggttttgttcctggactccagggactgctcggctagtgcacaatatgatgccgctggcaaaatatcgctcaactcggtccaagcatgggcggttaagcttggtactgagctgtatcacttcggagaatttatcaccaggaagaaagaggtgcaggatagttttaaatccgctcaagtggaatcgagagatggggaaaaactagttcagagtatgtcagacgacatacgcgccatgatggaacttaagatcagcgccgtcaaaaggatagtggaagctgctgaaaacatggcgttcgataaacaaaatgagcccgtacctgaagattttcagttctataatagtaaagaaatggaggaaccctgggacgaggtgtcaatcacaacaacgcctgagacagacttcggtcaggaaagttggatagtacgaccgccgtcaaagaatgcgcacacgcaacaaaacccacacttctcaaacattccagtaaacactaattttagcagtgtacatgttccaacaaatgtttatgcttgggcacctgaagttatcaagggaatccactggtcagagggactggacacgcacttcatcaataattaccagagcgacccgacgttatcgtggcaatacttcggcagctctactggattcatgagacattatcccgcattgaaatggagggctgaccctgtggatatttacgattgtcgaacgcgagcgtggtacatggaagctgcagcgagtccaaaggatgttatcattttagttgatcgtagcgggtcaatgaccgggcaaagacgggacatcgccaaacatgtcgttaccaatatactggatacgcttggcaacaacgattttgtcaacgttctaacttttgctgacaccgtagaagaaattgtaccgtgttttgaagattctctagtgcaggccactctgggaaatatacgggaactaaagttggcgatggacagttttgaaacgcaggagatagcaaacttttccgctgcgctgacgagagctttcgaactactggagatatatagaaataatagtggaggtgcaaattgcaaccaggctataatgctggtaacagatggcgtaccctacaattacaaagaaatatttgagaaatacaactggaagtatgagacccctgtgagagtgtttacgtacctgatagggcgtgaggtcgcagacgtaagagaggtgaagtggatggcgtgcgcgaatcgcgggttctacgtgcatctgagtactctagcggaggtgcgcgagcgcgtgttggaacatgtgaacgtgctcgcgcgcccgctcgtgctgcagagagaaaagcatcctgtcgtctggacacccgtctacgctaacgtcactgatccaaaagtagcagattatctgtgggagcaacgtgagcgagcagagcagaaagaacgcttcatgagccagcggagggacaagaatctcttcaactcggacaaagaacaggatagacgatggagaattactcagatgaaacagggccagtacagtgagctcggcaattcccagtatcagttaatgacatcagtgtccatgccggtgtacgacctccgacacaacgagtccgtagaggagaacggccagaaagagatgcgtattgctagactgttgggaatcgctggcactgacgtacctctttctgaaatacaagctctcatgacaccgtataagatcggtgtgaatggctacgcgtttatagtaacgaacaatggttacatcttgatacacccggatctgaggcctgtgttccaacaaatcttgaaaccaagctacaatagcgtcgatatgatagaagttgaactgtttgacgatgacagaggccccaggaactttagcaaagagctcactgcactccgaaaagaaattatcgatcaaaagacgggcaacaaggtgatgagtgtcaagtaccatttggatgatatgaaaagagtatcccgagcaaaaaggcattatttctggacgggtataagcgactcccccttcacgttggttgtggcaataccagagaattacggccgccaccgcatcacgccaccaccgactgatgatatccacaggctgtctctcacctccaaaaacatctcggcaaggcagtacctgtcggataagtggagcgttcatcctgactggatgtattgccggcactatgaacgcacgtttgcaactgctgaggaagagcttctctatttcttggagcgtgtagccaagcctggctggcgctggcctgccaagcctcgtcccccggagcaccacaagaacaaacctggacatgagcggcataacaacggaacaccagaaacgagagaaaggagcaaaatatctaatagcaccccaaggaatgagtattattgcgatcatggcttgatgcaggccttggtgtatgacgcgagaaatacagcctggtttaacaagagcatctctgattctgcatctgatgagaaagcggtggagtttattcaacggtttgggtacatcgtggcgttcctgtcaacccacagtggactaacgcgctggcagacccacccacctaaagaacatgataatgacaaaccagatttcggcaagcaatggccgcgagcgatcgaagaagtctggtaccgacgcgcagtcgagcagcactacgtagatcctttgagctatgtctacagtgtagacttgagcactgtcaagttccccctcaacgtaagcctggctctggtcaccgcatcacatgccgtcttccacggagacgggcataggaaggctccggcggctgtcgttgggtttcagttcaaacatgagcggctcagtgaatggtttgataatattacgtcttcgtgtgaacataacaaggagtgtacaacttgccagtctgacaactgggactgttacttggtggacaacaacggctgggttattgttagtgaagactccacacagactggacagttcttcggaaggattcgaccagacatcatgtcgagattagttgaagatgaagtctacaaggcagtgcatattgttgattaccaggcggtctgcttcagagagaagaaaactacgaacccagcgacaatgctctttaccccacttgaaaacttgcgcctcgtgatggcatggttcttagctaccacggtctggttctacaactccataacgacaggtttggctcaagcatccagctactccttcgatgacgagaatgttacacccactgttgcataccagagctacgaaaacgatgaagagacggatgacccatacatgtcgaaacctccaaacatgagggtcttagaacgagactatgagaaactggtgcttataaacaggacgcgaccaactccttgcgacagagagatgtacttgtaccagctagactataacaacttagacgagaagctgaacaagccagtggcggagtgcgggcgaccattcatagctcaacacgtcaactatacaaacatgctaatgattgtggtagacgccaattgcccgaaagacgaggtcgcagccatgtcggtggatgcgacagaagtgaactataacgagtcgctgccttgcctgaagcatatgcatcccttgtataggaagcagcctacttcctgcataaggaatcatactgaggagagcaacatcgacatgtgcggacgcggcagtctacccggcaacagcaagacatggatttcgctgagcatctttttactagtcaaatattatctggcgtaaagtgtaggttcaaagcttagagaggtaaagcacaaagcggccagactggggaatgaaggtgcaatatgccagggtaggaataaatataaattttctggctgcctattctttatctctctggtattaccgttaaagaaaacctcactgaaatatcagccattcttaggtaaatttttaaataatgcatacgataagtcggagagccgggtattgttttaaatggatattgaggcctggaactgcgagcgggcaaagacgtggtttcaggcctaggtaatagatttaattatacgttttgttaaaatgtgtcatagtttatcggaatatttttcgtagtaggtaaatccaaagatatatcgatggtaacaacttttaattataatcgtttcttttgtttgattattattataattgtattgatctccgcttttagaataggtagatattataaaaaatacatgtcattcccacatatcaaattttaaatgatacgagtatgctcccattgtgccacaataggaagcaatataaaaatatcatatcaaaaattcaattacccccgtggtatgtgggggtcaactttttaaaaattgtgaacaaaataaaaaaaattatagtataaaattatatagcttttgtaaaacgtttggctgcatgattttaaatttccctttatttatgtaatatttagatcttttctttatttccatatctaaatgttaaattatttccgtcctgctggtgaaaaggattcacttattaatttggacaatatgcaaaatgacaaacaattagggcaaatcttcaaattaatttgtttgcattgaaccagtaggtattacaccactggttgaaataaatatttgtacctaatattactagaatttagtatttcgaatctctgtcatttaagtatttgtcttagaagtcaactatttactgaattacctactgaaatgtgattgtttgtttgtaataaataaatgattattaaaagtattcaaaaaaaaaaaaaaaa

SEQ ID NO: 18 - protein, 1258 aa, Heliothis virescens
MCKCVGAFVLLLLVLFLDSRDCSASAQYDAAGKISLNSVQAWAVKLGTELYHFGEFITRKKEVQDSFKSAQVESRDGEKLVQSMSDDIRAMMELKISAVKRIVEAAENMAFDKQNEPVPEDFQFYNSKEMEEPWDEVSITTTPETDFGQESWIVRPPSKNAHTQQNPHFSNIPVNTNFSSVHVPTNVYAWAPEVIKGIHWSEGLDTHFINNYQSDPTLSWQYFGSSTGFMRHYPALKWRADPVDIYDCRTRAWYMEAAASPKDVIILVDRSGSMTGQRRDIAKHVVTNILDTLGNNDFVNVLTFADTVEEIVPCFEDSLVQATLGNIRELKLAMDSFETQEIANFSAALTRAFELLEIYRNNSGGANCNQAIMLVTDGVPYNYKEIFEKYNWKYETPVRVFTYLIGREVADVREVKWMACANRGFYVHLSTLAEVRERVLEHVNVLARPLVLQREKHPVVWTPVYANVTDPKVADYLWEQRERAEQKERFMSQRRDKNLFNSDKEQDRRWRITQMKQGQYSELGNSQYQLMTSVSMPVYDLRHNESVEENGQKEMRIARLLGIAGTDVPLSEIQALMTPYKIGVNGYAFIVTNNGYILIHPDLRPVFQQILKPSYNSVDMIEVELFDDDRGPRNFSKELTALRKEIIDQKTGNKVMSVKYHLDDMKRVSRAKRHYFWTGISDSPFTLVVAIPENYGRHRITPPPTDDIHRLSLTSKNISARQYLSDKWSVHPDWMYCRHYERTFATAEEELLYFLERVAKPGWRWPAKPRPPEHHKNKPGHERHNNGTPETRERSKISNSTPRNEYYCDHGLMQALVYDARNTAWFNKSISDSASDEKAVEFIQRFGYIVAFLSTHSGLTRWQTHPPKEHDNDKPDFGKQWPRAIEEVWYRRAVEQHYVDPLSYVYSVDLSTVKFPLNVSLALVTASHAVFHGDGHRKAPAAVVGFQFKHERLSEWFDNITSSCEHNKECTTCQSDNWDCYLVDNNGWVIVSEDSTQTGQFFGRIRPDIMSRLVEDEVYKAVHIVDYQAVCFREKKTTNPATMLFTPLENLRLVMAWFLATTVWFYNSITTGLAQASSYSFDDENVTPTVAYQSYENDEETDDPYMSKPPNMRVLERDYEKLVLINRTRPTPCDREMYLYQLDYNNLDEKLNKPVAECGRPFIAQHVNYTNMLMIVVDANCPKDEVAAMSVDATEVNYNESLPCLKHMHPLYRKQPTSCIRNHTEESNIDMCGRGSLPGNSKTWISLSIFLLVKYYLA

SEQ ID NO: 19 - DNA, 35 bp, Artificial
  PCR Primer
  misc_feature (35)..(35): n is a, c, g or t
gagagagagaagagctcttttttttttttttttvn

SEQ ID NO: 20 - DNA, 20 bp, Artificial
  PCR Primer
tcgagctgaggtacgaacgc

SEQ ID NO: 21 - DNA, 17 bp, Artificial
  PCR Primer
  misc_feature (1)..(1): n is pseudouridine
ngcgttcgtacctcagc

SEQ ID NO: 22 - DNA, 33 bp, Artificial
  PCR Primer
  misc_feature (25)..(25): n is a, g, c or t
  misc_feature (31)..(31): n is a, g, c or t
gggctacaccatcagaatcctgytntggacntt

SEQ ID NO: 23 - protein, 11 aa, Artificial
  peptide
GYTIRILLWTF

SEQ ID NO: 24 - DNA, 32 bp, Artificial
  PCR Primer
gagtctctggtcaggtagtcgaarttrtccat

SEQ ID NO: 25 - protein, 11 aa, Artificial
  peptide
MDNFDYLTRDS

SEQ ID NO: 26 - DNA, 20 bp, Artificial
  PCR Primer
gatacgaagtaggcgtaggc

SEQ ID NO: 27 - protein, 6 aa, Artificial
  peptide
AYAYFV

SEQ ID NO: 28 - DNA, 20 bp, Artificial
  PCR Primer
cgatgagaaggcacacgtag

SEQ ID NO: 29 - protein, 6 aa, Artificial
  peptide
YVCLLI

SEQ ID NO: 30 - DNA, 18 bp, Artificial
  PCR Primer
atttaggtgacactatag

SEQ ID NO: 31 - DNA, 20 bp, Artificial
  PCR Primer
ctacgtgtgccttctcatcg

SEQ ID NO: 32 - DNA, 20 bp, Artificial
  PCR Primer
gcctacgcctacttcgtatc

SEQ ID NO: 33 - DNA, 20 bp, Artificial
  PCR Primer
taatacgactcactataggg

SEQ ID NO: 34 - DNA, 24 bp, Artificial
  PCR Primer
cgtcgggaagatatacgctggact

SEQ ID NO: 35 - protein, 8 aa, Artificial
  peptide
VGKIYAGL

SEQ ID NO: 36 - DNA, 24 bp, Artificial
  PCR Primer
cgtcagcaacgtcgtcgagatagt

SEQ ID NO: 37 - protein, 8 aa, Artificial
  peptide
VSNVVEIV

SEQ ID NO: 38 - DNA, 45 bp, Artificial
  PCR Primer
ctaatacgactcactatagggcaagcagtggtatcaacgcagagt

SEQ ID NO: 39 - DNA, 22 bp, Artificial
  PCR Primer
ctaatacgactcactatagggc

SEQ ID NO: 40 - DNA, 23 bp, Artificial
  PCR Primer
aagcagtggtatcaacgcagagt

SEQ ID NO: 41 - DNA, 24 bp, Artificial
  PCR Primer
aagtactggtcatcgctccggaac

SEQ ID NO: 42 - protein, 8 aa, Artificial
  peptide
KYWSSLRN

SEQ ID NO: 43 - DNA, 24 bp, Artificial
  PCR Primer
gttccttgctatcgctgtccacaa

SEQ ID NO: 44 - protein, 8 aa, Artificial
  peptide
FLAIAVDN

SEQ ID NO: 45 - DNA, 24 bp, Artificial
  PCR Primer
cgacccttggcagtcttcaaaagt

SEQ ID NO: 46 - protein, 8 aa, Artificial
  peptide
TEFDCQGS

SEQ ID NO: 47 - DNA, 24 bp, Artificial
  PCR Primer
gacagattctgccccgcacccttt

SEQ ID NO: 48 - protein, 8 aa, Artificial
  peptide
KGAGQNLS

SEQ ID NO: 49 - DNA, 26 bp, Artificial
  PCR Primer
  misc_feature (18)..(18): n is a, c, g or t
  misc_feature (24)..(24): n is a, c, g or t
tccggcatcccctccytncargtngt

SEQ ID NO: 50 - protein, 9 aa, Artificial
  peptide
SGIPSLQVV

SEQ ID NO: 51 - DNA, 24 bp, Artificial
  PCR Primer
tatcaccaggttccggagcgatga

SEQ ID NO: 52 - protein, 8 aa, Artificial
  peptide
SSLRNLVI

SEQ ID NO: 53 - DNA, 24 bp, Artificial
  PCR Primer
ctcagaccacacgacctcgaagat

SEQ ID NO: 54 - protein, 8 aa, Artificial
  peptide
IFEVVWSE

SEQ ID NO: 55 - DNA, 24 bp, Artificial
  PCR Primer
aaactcacgttgtccgcgttacac

SEQ ID NO: 56 - protein, 8 aa, Artificial
  peptide
CNADNVSL

SEQ ID NO: 57 - DNA, 24 bp, Artificial
  PCR Primer
ggccgatgatagcgaagatgacta

SEQ ID NO: 58 - protein, 8 aa, Artificial
  peptide
IVIFAIIG

SEQ ID NO: 59 - DNA, 24 bp, Artificial
  PCR Primer
cctcggtcttttccagattctgtg

SEQ ID NO: 60 - protein, 8 aa, Artificial
  peptide
AQNLEKTE

SEQ ID NO: 61 - DNA, 24 bp, Artificial
  PCR Primer
ggtcttatcgccgttaggcaaatg

SEQ ID NO: 62 - protein, 8 aa, Artificial
  peptide
HLPNGDKT

SEQ ID NO: 63 - DNA, 24 bp, Artificial
  PCR Primer
gctgatggcgatgaatgaacactg

SEQ ID NO: 64 - DNA, 35 bp, Artificial
  PCR Primer
cgcggatccgaacactgcgtttgctggctttgatg

SEQ ID NO: 65 - DNA, 29 bp, Artificial
  PCR Primer
  misc_feature (27)..(27): n is a, c, g or t
caggagatcgagcgcatcttygarytngc

SEQ ID NO: 66 - protein, 9 aa, Artificial
  peptide
QEIERIFEL

SEQ ID NO: 67 - DNA, 28 bp, Artificial
  PCR Primer
  misc_feature (20)..(20): n is a, c, g or t
gctggttctcgtccaggatnacrtcraa

SEQ ID NO: 68 - protein, 9 aa, Artificial
  peptide
FDVILDENQ

SEQ ID NO: 69 - DNA, 20 bp, Artificial
  PCR Primer
tcggactctccaattggtag

SEQ ID NO: 70 - protein, 6 aa, Artificial
  peptide
RTLQLV

SEQ ID NO: 71 - DNA, 20 bp, Artificial
  PCR Primer
catgtcaggcgggcactgcg

SEQ ID NO: 72 - protein, 7 aa, Artificial
  peptide
AQCPPDM

SEQ ID NO: 73 - DNA, 22 bp, Artificial
  PCR Primer
tgggaagttcttcggtaaagta

SEQ ID NO: 74 - DNA, 19 bp, Artificial
  PCR Primer
ggcctggcacacatagatt

SEQ ID NO: 75 - DNA, 21 bp, Artificial
  PCR Primer
cgggattgctacatacttgac

SEQ ID NO: 76 - DNA, 22 bp, Artificial
  PCR Primer
tttgtgacaacccaacgaatta

SEQ ID NO: 77 - DNA, 23 bp, Artificial
  PCR Primer
  misc_feature (15)..(15): n is a, c, g or t
  misc_feature (18)..(18): n is a, c, g or t
gtgcagacctgggcngmnaaryt

SEQ ID NO: 78 - DNA, 27 bp, Artificial
  PCR Primer
gccgacgatcatgatggcytgrttrca

SEQ ID NO: 79 - DNA, 21 bp, Artificial
  PCR Primer
gcgccatgatggaacttaaga

SEQ ID NO: 80 - DNA, 21 bp, Artificial
  PCR Primer
tgccgaagtactgccacgaca

SEQ ID NO: 81 - protein, 8 aa, Artificial
  peptide
VQTWAEKL

SEQ ID NO: 82 - protein, 9 aa, Artificial
  peptide
CNQAIMIVS

SEQ ID NO: 83 - protein, 11 aa, Artificial
  peptide
PLTKVIGLLPR